(19) United States
(12) Patent Application Publication
(10) Pub. No.: US 2024/0191297 A1
(43) Pub. Date: Jun. 13, 2024

(54) METHODS, COMPOSITIONS, AND SYSTEMS FOR ASSESSING BIOLOGICAL SAMPLE QUALITY

(71) Applicant: 10x Genomics, Inc., Pleasanton, CA (US)

(72) Inventors: Cheyenne CHRISTOPHERSON, San Ramon, CA (US); Veronica Emelina GONZALEZ MUÑOZ, Palo Alto, CA (US); Joshua GU, Dublin, CA (US); Albert Dale KIM, Oakland, CA (US); Yi LUO, Dublin, CA (US); Meiliana TJANDRA, Dublin, CA (US)

(21) Appl. No.: 18/486,918

(22) Filed: Oct. 13, 2023

Related U.S. Application Data
(60) Provisional application No. 63/416,451, filed on Oct. 14, 2022.

(51) Int. Cl. C12Q 1/6881; C12Q 1/6806; C12Q 1/6841

(52) U.S. Cl. CPC C12Q 1/6881; C12Q 1/6806; C12Q 1/6841; C12Q 2600/166

(57) ABSTRACT

The present disclosure relates in some aspects to methods and compositions for quality control (e.g., assessing the level of fixation of a biological sample) and/or optimization of analyte detection in fixed samples. In some embodiments, the method can be used to process a fixed biological sample, the method comprising contacting the fixed biological sample with a nucleic acid stain and/or an actin stain; detecting an optical signal associated with the nucleic acid stain and/or an optical signal associated with the actin stain; comparing the detected optical signal(s) to one or more references to determine the quality of the biological sample. Single cell analysis, spatial array based analysis or in situ analysis can be performed using the processed biological sample.

### CROSS-REFERENCE TO RELATED APPLICATIONS

This application claims priority to U.S. Provisional Patent Application No. 63/416,451, filed Oct. 14, 2022, entitled “METHODS, COMPOSITIONS, AND SYSTEMS FOR ASSESSING BIOLOGICAL SAMPLE QUALITY” which is herein incorporated by reference in its entirety for all purposes.

### FIELD

The present disclosure relates in some aspects to methods for assessing level of fixation in fixed biological samples for optimal detection of analytes in the samples.

### BACKGROUND

Biological sample preparation procedures remain highly variable from sample to sample and a persistent source of concern for downstream assay performance. Methods for assessing optimal sample preparation (e.g., fixation) of biological samples and the quality of the samples for downstream workflows such as in situ detection, analysis using a spatial array and single cell analysis are lacking, including qualitative detection of under-fixation and over-fixation of biological samples. Efficient and effective assessment of sample preparation (e.g., fixation) may enhance tissue preparation, including refining sample fixation time, identifying treatment conditions, and related downstream processing. Accordingly, there remains a need to optimize sample processing in a fast, sensitive, and selective manner, in particular, for subsequent analyte detection in under-fixed or over-fixed biological samples. The present disclosure addresses such and other needs.

### SUMMARY

In some embodiments, provided herein is a method for sample processing and/or analysis, comprising: contacting a fixed biological sample with a nucleic acid stain and/or an actin stain; detecting an optical signal associated with the nucleic acid stain and/or an optical signal associated with the actin stain in the fixed biological sample; and comparing the detected optical signal(s) to a reference. In some embodiments, the method further comprises de-crosslinking or additionally fixing the fixed biological sample to adjust the level of fixation; and/or contacting the fixed biological sample with a nucleic acid probe that directly or indirectly binds to an analyte or a product thereof in the fixed biological sample. The step of de-crosslinking or additionally fixing the fixed biological sample to adjust the level of fixation can be based on the comparison of the detected optical signal(s) to the reference. In some embodiments, the method can comprise providing the fixed biological sample prior to the contacting it with the nucleic acid stain and/or the actin stain.

In any of the embodiments herein referring to an analyte or product thereof, the product thereof can be an extension, amplification, and/or ligation product of the analyte. In some embodiments, the product thereof is a cDNA product of an RNA analyte.

In some embodiments, provided herein is a method for sample processing and/or analysis, comprising: contacting a fixed biological sample with a nucleic acid stain and/or an actin stain; detecting an optical signal associated with the nucleic acid stain and/or an optical signal associated with the actin stain in the fixed biological sample; and comparing the optical signal(s) detected in b) to a reference to determine the quality of the sample. Based on the assessment of sample quality, in some embodiments, the method can further comprise de-crosslinking or additionally fixing the fixed biological sample to adjust the level of fixation.

Based on the assessment of sample quality, in some embodiments, the method can further comprise contacting the fixed biological sample with a nucleic acid probe that directly or indirectly binds to an analyte or a product thereof in the fixed biological sample, e.g., without adjusting the level of sample fixation prior to contacting with the nucleic acid probe. In some embodiments, the method can further comprise de-crosslinking or additionally fixing the fixed biological sample to adjust the level of fixation, and contacting the fixed biological sample having an adjusted level of fixation with a nucleic acid probe that directly or indirectly binds to an analyte or a product thereof in the fixed biological sample.

Based on the assessment of sample quality, in some embodiments, the method can further comprise adjusting the level of fixation of an additional biological sample. For instance, in cases where the fixed biological sample is not or cannot be de-crosslinked or additionally fixed, a suggestion can be made (e.g., based on the assessed sample quality of the fixed biological sample) to alter sample processing of the additional biological sample which is subsequently used for analyte detection. In some embodiments, the fixed biological sample and the additional biological sample are of the same cell type or the same tissue type. In some embodiments, the fixed biological sample and the additional biological sample are portions (e.g., sections) of the same cell or tissue sample (e.g., a tissue block). In some embodiments, the fixed biological sample and the additional biological sample are consecutive sections of the same tissue block. In some embodiments, the method further comprises contacting the additional biological sample (e.g., having an adjusted level of fixation) with a nucleic acid probe that directly or indirectly binds to an analyte or a product thereof in the additional biological sample.

In any of the embodiments herein, the nucleic acid stain can be cell-permeant. In some embodiments, cells in the fixed biological sample may but do not need to be permeabilized during or prior to contacting with the nucleic acid stain and/or the actin stain. In any of the embodiments herein, the nucleic acid stain may be non-fluorescent or substantially non-fluorescent in the absence of nucleic acids. In any of the embodiments herein, the nucleic acid stain can comprise one or more stains that are fluorescent when bound to RNA, such as a SYTO™ RNASelect™ stain, a RNA integrity and quality (IQ) dye (e.g., Qubit™ RNA IQ kit), a SYTO™ 64 stain, a SYTO™ 647 stain, a SYTO™ 17 stain, or a SYTO™ 63 stain. In any of the embodiments herein, the nucleic acid stain can selectively bind to RNA over DNA. In any of the embodiments herein, the nucleic acid stain can comprise a dye that binds to intact RNA and/or a dye that binds to degraded RNA. The intact RNA may comprise mRNA, tRNA, and/or rRNA. In any of the embodiments herein, the nucleic acid stain may but does not need to comprise DAPI, propidium iodide (PI), a Hoechst stain, and/or a fluorescent Nissl stain.

In any of the embodiments herein, the nucleic acid stain can comprise a quinolinium scaffold. In any of the embodiments herein, the nucleic acid stain can comprise an aminoethylpiperidine group. In any of the embodiments herein, the nucleic acid stain can comprise (E)-2-(2-(1H-indol-3-yl)vinyl)-1-methylquinolin-1-ium iodide, (E)-2-(2-(1H-indol-2-yl)vinyl)-1-methyl-4-((2-(piperidin-1-yl)ethyl)amino) quinolin-1-ium iodide, or (E)-2-(2-(1H-indol-3-yl)vinyl)-1-methyl-4-((2-(piperidin-1-yl)ethyl)amino) quinolin-1-ium iodide.

In any of the embodiments herein, the actin stain can be fluorescent. In any of the embodiments herein, the actin stain can be conjugated to a fluorescent moiety. In any of the embodiments herein, the actin stain can selectively bind to polymeric actin over monomeric actin. In any of the embodiments herein, the actin stain can selectively bind to F-actin. In any of the embodiments herein, the actin stain can comprise phalloidin or a derivative thereof. In any of the embodiments herein, the actin stain can comprise an anti-actin antibody or an epitope-binding fragment thereof.

In any of the embodiments herein, the fixed biological sample can be contacted with the nucleic acid stain and the actin stain. In any of the embodiments herein, the fixed biological sample can be contacted with the nucleic acid stain prior to, simultaneously with, or after contacting with the actin stain. The nucleic acid stain and the actin stain can be the same composition or in separate compositions that are separately contacted with a fixed sample simultaneously or in any order.

In any of the embodiments herein, the fixed biological sample can be fixed using a fixing composition. In any of the embodiments herein, the fixing composition can comprise one or more crosslinking agents. In any of the embodiments herein, the fixing composition can comprise 0.01-100% of a fixative selected from the group consisting of: formaldehyde, glutaraldehyde, acetone, methanol, ethanol, acetic acid, potassium dichromate, chromic acid, potassium permanganate, B-5, Zenker's fixative, uranyl acetate, mercuric chloride, osmium tetroxide, potassium permanganate, and 1-ethyl-3-(3-dimethylamino propyl)carbodiimide (EDC), picric acid, glyoxal, bis(sulfosuccinimidyl)suberate, and derivatives thereof. In any of the embodiments herein, the fixing composition can be free or substantially free of an alcohol. In any of the embodiments herein, the fixing composition can be free or substantially free of methanol and ethanol. In any of the embodiments herein, the fixed biological sample can be fixed using a neutral buffered formalin (NBF) or a paraformaldehyde (PFA) solution.

In any of the embodiments herein, the fixed biological sample can be immobilized on a substrate. In some embodiments, the substrate comprises a planar surface for sample contact prior to, during, and/or after fixing a biological sample to provide the fixed biological sample. In any of the embodiments herein, the substrate can be a solid substrate. In some embodiments, the substrate does not comprise a bead, particle, or microwell. In any of the embodiments herein, the substrate can be a planar substrate. In any of the embodiments herein, the substrate can be a glass slide or a plastic slide. In any of the embodiments herein, the substrate can be transparent. In any of the embodiments herein, the substrate can but does not need to comprise nucleic acid immobilized thereon prior to contacting the fixed biological sample.

In any of the embodiments herein, the fixed biological sample can be a tissue section. In any of the embodiments herein, the fixed biological sample can be about 5 μm, about 10 μm, about 20 μm, about 30 μm, about 40 μm, or about 50 μm in thickness. In any of the embodiments herein, the tissue section can be a normal tissue section or associated with a disease or condition. In any of the embodiments herein, the tissue section can comprise one or more cancer cells, one or more stem cells, one or more immune cells, one or more apoptotic cells, one or more necrotic cells, and/or one or more pathogens. In any of the embodiments herein, the fixed biological sample can comprise dissociated cells, cultured cells, and/or cells isolated from a subject. In any of the embodiments herein, the fixed biological sample can be a freshly isolated biological sample that is fixed. In any of the embodiments herein, the fixed biological sample can be a frozen biological sample that is thawed and fixed. In any of the embodiments herein, the fixed biological sample can be a fresh frozen biological sample that is fixed. In any of the embodiments herein, the fixed biological sample can be an archived sample. In any of the embodiments herein, the fixed biological sample can be a paraffin-embedded biological sample. In any of the embodiments herein, the fixed biological sample can be a formalin-fixed paraffin-embedded (FFPE) biological sample. In any of the embodiments herein, the fixed biological sample can be de-paraffinizing prior to the contacting with the nucleic acid stain and/or the actin stain. In some embodiments, the de-paraffinizing comprises contacting the fixed biological sample with xylene, ethanol, and water. In some embodiments, the de-paraffinizing comprises sequentially contacting the fixed biological sample with xylene, absolute ethanol, about 96% ethanol, about 70% ethanol, and water.

In any of the embodiments herein, the fixed biological sample may but does no need to be de-crosslinked prior to or during the contacting with the nucleic acid stain and/or the actin stain. In any of the embodiments herein, the fixed biological sample may be de-crosslinked after the comparing. In some embodiments, prior to or during the contacting in a), the method does not comprise contacting the fixed biological sample with a catalyst that catalyzes de-crosslinking of molecular crosslinks. In any of the embodiments herein, the de-crosslinking can comprise contacting the fixed biological sample with a catalyst that catalyzes de-crosslinking of molecular crosslinks. In some embodiments, the catalyst non-enzymatically catalyzes de-crosslinking of inter-molecular crosslinks and/or intra-molecular crosslinks in the fixed biological sample. In some embodiments, the inter-molecular crosslinks and/or intra-molecular crosslinks comprise an aminal bridge.

In any of the embodiments herein, the fixed biological sample can be additionally fixed after the comparing. In some embodiments, the additional fixation comprises contacting the fixed biological sample with a crosslinking agent. In any of the embodiments herein, the fixed biological sample can be additionally fixed using an additional fixing composition which optionally comprises 0.01-100% of a fixative selected from the group consisting of: formaldehyde, glutaraldehyde, acetone, methanol, ethanol, acetic acid, potassium dichromate, chromic acid, potassium permanganate, B-5, Zenker's fixative, uranyl acetate, mercuric chloride, osmium tetroxide, potassium permanganate, and 1-ethyl-3-(3-dimethylamino propyl)carbodiimide (EDC), picric acid, and derivatives thereof. The fixing composition for providing the fixed biological sample and the additional fixing composition can be the same or different. In some embodiments, the additional fixing composition comprises an alcohol, whereas the fixing composition for providing the fixed biological sample does not comprise an alcohol. In some embodiments, the additional fixing composition comprise methanol or ethanol, whereas the fixing composition for providing the fixed biological sample does not comprise methanol or ethanol. In any of the embodiments herein, the additional fixing composition can be free or substantially free of an alcohol and can comprise a neutral buffered formalin (NBF) or a paraformaldehyde (PFA) solution.

In some embodiments, the fixed biological sample is neither de-crosslinked nor additionally fixed after the comparing. In some embodiments, the fixed biological sample is neither de-crosslinked nor additionally fixed prior to contacting with a nucleic acid probe that directly or indirectly binds to an analyte in the fixed biological sample.

In any of the embodiments herein, the nucleic acid stain can be a first nucleic acid stain, and the detecting the optical signal(s) associated with the nucleic acid stain and/or the actin stain can comprise detecting an optical signal associated with a second nucleic acid stain. In some embodiments, the first nucleic acid stain selectively binds to RNA and the second nucleic acid stain selectively binds to DNA. In any of the embodiments herein, the second nucleic acid stain can comprise DAPI, propidium iodide (PI), a Hoechst stain, or a fluorescent Nissl stain. In any of the embodiments herein, the optical signal associated with the first nucleic acid and the optical signal associated with the second nucleic acid can be detected in a nucleus. In any of the embodiments herein, the optical signals can be detected in the nuclei of cells in the fixed biological sample. In any of the embodiments herein, the comparing can comprise using a ratio between the optical signal associated with the first nucleic acid and the optical signal associated with the second nucleic acid in the fixed biological sample. In any of the embodiments herein, the comparing can comprise using a ratio between the optical signal associated with an RNA stain that selectively binds to RNA over DNA and the optical signal associated with a DNA stain that selectively binds to DNA over RNA.

In any of the embodiments herein, the comparing can comprise using a ratio between the optical signal associated with the nucleic acid stain and an optical signal associated with background signal detected in a cytoplasm or non-nucleic area in the fixed biological sample. In some embodiments, the nucleic acid stain selectively binds to RNA. In any of the embodiments herein, the optical signal associated with the nucleic acid stain can be detected in a nucleus. In any of the embodiments herein, the fixed biological sample can be contacted with an additional nucleic acid stain or a cytoplasm stain for determining the location of the nucleus. In any of the embodiments herein, the additional nucleic acid stain can selectively bind to DNA. In any of the embodiments herein, the additional nucleic acid stain can comprise DAPI.

In any of the embodiments herein, the comparing can comprise using a ratio between the optical signal associated with the actin stain and an optical signal associated with an additional nucleic acid stain in the fixed biological sample. In some embodiments, the additional nucleic acid stain selectively binds to DNA. In some embodiments, the additional nucleic acid stain is DAPI. In any of the embodiments herein, the optical signal associated with the actin stain can be detected in a cytoplasm and the optical signal associated with the additional nucleic acid stain can be detected in a nucleus.

In any of the embodiments herein, the comparing can comprise using an optical signal associated with the nucleic acid stain, an optical signal associated with the actin stain, an optical signal associated with an additional nucleic acid stain, and/or an optical signal associated with an additional actin stain in a reference sample. For example, the comparing can comprise quantitatively and/or qualitatively comparing optical signals in on image of the fixed biological sample to optical signals in one or more other images of the fixed biological sample or a reference sample different from the fixed biological sample. In any of the embodiments herein, an elevated level of the nucleic acid stain and/or the additional nucleic acid stain in the fixed biological sample compared to that in the reference sample can indicate the fixed biological sample is less fixed than the reference sample. In any of the embodiments herein, a decreased level of the nucleic acid stain and/or the additional nucleic acid stain in the fixed biological sample compared to that in the reference sample can indicate the fixed biological sample is more fixed than the reference sample. In any of the embodiments herein, an elevated level of the actin stain and/or the additional actin stain in the fixed biological sample compared to that in the reference sample can indicate the fixed biological sample is more fixed than the reference sample. In any of the embodiments herein, a decreased level of the actin stain and/or the additional actin stain in the fixed biological sample compared to that in the reference sample can indicate the fixed biological sample is less fixed than the reference sample.

In any of the embodiments herein, based on the comparing, the fixed biological sample can be neither over-fixed nor under-fixed and do not need to be de-crosslinked or additionally fixed; or the fixed biological sample can be over-fixed and can be de-crosslinked to provide a de-crosslinked fixed biological sample; or the fixed biological sample can be under-fixed and can be additionally fixed to provide an additionally fixed biological sample. In any of the embodiments herein, the method can comprise permeabilizing the fixed biological sample (which is neither de-crosslinked nor additionally fixed), the de-crosslinked fixed biological sample, or the additionally fixed biological sample. In any of the embodiments herein, the method can comprise contacting a cell or nucleus in the fixed biological sample (which is neither de-crosslinked nor additionally fixed), a cell or nucleus in the de-crosslinked fixed biological sample, or a cell or nucleus in the additionally fixed biological sample with the nucleic acid probe that directly or indirectly binds to the analyte in the cell or nucleus.

In any of the embodiments herein, the method can comprise detecting an optical signal associated with the nucleic acid probe or a product thereof at a location of the cell or nucleus, thereby detecting the analyte at the location in the fixed biological sample (which is neither de-crosslinked nor additionally fixed), the de-crosslinked fixed biological sample, or the additionally fixed biological sample.

In any of the embodiments herein, the method can comprise partitioning the cell or nucleus in a partition comprising a partition barcode. The partition can be an emulsion droplet or a microwell. In any of the embodiments herein, the partition can be a single-cell partition containing only one cell or nuclei. In any of the embodiments herein, the method can comprise sequencing a nucleic acid molecule or a portion thereof comprising i) a sequence of the nucleic acid probe or a complement thereof and ii) the partition barcode or a complement thereof.

In any of the embodiments herein, the nucleic acid stain and/or the actin stain can but do not need to be removed after the comparing, and prior to the de-crosslinking or additionally fixing and/or prior to the contacting with the nucleic acid probe. In any of the embodiments herein, the nucleic acid stain and/or the actin stain may not need to be removed.

In any of the embodiments herein, prior to, during, and/or after the de-crosslinking or additionally fixing and/or the contacting with the nucleic acid probe, the nucleic acid stain and/or the actin stain can be removed by a buffer comprising a salt, a divalent cation, a denaturing agent, an ionic detergent, and/or a non-ionic detergent. In some embodiments, the nucleic acid stain and/or the actin stain are removed after the contacting with the nucleic acid probe and the buffer comprises saline-sodium citrate (SSC) and/or formamide. In some embodiments, the nucleic acid stain and/or the actin stain are removed by using a buffer comprises SSC, for instance, during nucleic acid probe hybridization. In any of the embodiments herein, the method can comprise removing unbound and nonspecifically bound nucleic acid probe, wherein the nucleic acid stain and/or the actin stain are removed together with the unbound and nonspecifically bound nucleic acid probe.

In any of the embodiments herein, the nucleic acid probe can comprise a detectable label. In some embodiments, the detectable label comprises a nucleic acid sequence or an optically detectable label. In any of the embodiments herein, the nucleic acid probe can comprise a barcode region comprising one or more barcode sequences. In any of the embodiments herein, the analyte can be a cellular nucleic acid. In some embodiments, the cellular nucleic acid is genomic DNA, RNA, or cDNA.

In any of the embodiments herein, the nucleic acid probe can be a primary probe that hybridizes to the cellular nucleic acid. In any of the embodiments herein, the primary probe can comprise a barcode sequence corresponding to the cellular nucleic acid or a portion thereof. In any of the embodiments herein, the primary probe can be selected from the group consisting of: a primary probe comprising a 3′ or 5′ overhang upon hybridization to the cellular nucleic acid; a primary probe comprising a 3′ overhang and a 5′ overhang upon hybridization to the cellular nucleic acid; a circular primary probe; a circularizable primary probe or probe set; a primary probe or probe set comprising a split hybridization region configured to hybridize to a splint; and a combination thereof. In any of the embodiments herein, the 3′ overhang and/or the 5′ overhang can each independently comprise one or more barcode sequences. In any of the embodiments herein, the split hybridization region can comprise one or more barcode sequences.

In any of the embodiments herein, the cellular nucleic acid can be an mRNA, and a first nucleic acid probe and a second nucleic acid probe can be hybridized to a first analyte sequence and a second analyte sequence in the mRNA, respectively, wherein: the first nucleic acid probe comprises: i) a first hybridization region complementary to the first analyte sequence, and ii) a first overhang; the second nucleic acid probe comprises: i) a second hybridization region complementary to the second analyte sequence, and ii) a second overhang; the first and second nucleic acid probes are ligated using the mRNA as template to form a ligated nucleic acid probe, with or without gap filling prior to the ligation; and the first and second overhangs independently comprise a primer binding sequence, a capture sequence, a barcode sequence, and/or a constant sequence. The constant sequence can be a common sequence among a plurality of nucleic acid probes targeting the same or different analytes such as mRNAs. In some embodiments, the first overhang is a 5′ overhang comprising a primer binding sequence, and the second overhang is a 3′ overhang comprising a probe barcode sequence, a constant sequence, and a capture sequence on the 3′ end. In some embodiments, the capture sequence is complementary to a capture probe comprising a partition barcode, a primer binding sequence, and an UMI. In some embodiments, the capture probe is immobilized on a bead or a microwell of a partition. In some embodiments, upon hybridization of the capture sequence to the capture probe, the method comprises generating in the partition a nucleic acid molecule comprising the partition barcode, the UMI, a complement of the probe barcode sequence, the first analyte sequence, and the second analyte sequence. As such, in some embodiments, by determining the sequences of the nucleic acid molecules generated in the partitions, each analyte sequence (e.g., the first and second analyte sequences on the mRNA) and sequences of its associated probe barcode and partition barcode can be determined, and a plurality of analytes can be detected in partitioned cells (e.g., cells in single-cell partitions) from the biological sample.

In any of the embodiments herein, the nucleic acid probe can be a detectable probe that hybridizes to a primary probe or a product or complex thereof, wherein the primary probe hybridizes to the cellular nucleic acid. In any of the embodiments herein, the product or complex of the primary probe can be selected from the group consisting of: a rolling circle amplification (RCA) product, a complex comprising an initiator and an amplifier for hybridization chain reaction (HCR), a complex comprising an initiator and an amplifier for linear oligonucleotide hybridization chain reaction (LO-HCR), a primer exchange reaction (PER) product, and a complex comprising a pre-amplifier and an amplifier for branched DNA (bDNA). In any of the embodiments herein, the detectable probe can hybridize to a barcode sequence in the primary probe or product or complex thereof. In any of the embodiments herein, the detectable probe can comprise a barcode sequence in a region that does not hybridize to the primary probe or product or complex thereof. In any of the embodiments herein, the detectable probe can be selected from the group consisting of: a detectable probe comprising a 3′ or 5′ overhang upon hybridization to the primary probe or product or complex thereof, a detectable probe comprising a 3′ overhang and a 5′ overhang upon hybridization to the primary probe or product or complex thereof; a circular detectable probe; a circularizable detectable probe or probe set; a detectable probe or probe set comprising a split hybridization region configured to hybridize to a splint; and a combination thereof. In any of the embodiments herein, the 3′ overhang and/or the 5′ overhang can each independently comprise one or more barcode sequences. In any of the embodiments herein, the split hybridization region can comprise one or more barcode sequences. In any of the embodiments herein, the detectable probe can comprise a fluorescent label and/or a nucleic acid sequence for binding to a fluorescently labelled probe.

In any of the embodiments herein, the analyte can comprise a non-nucleic acid moiety, In some embodiments, the non-nucleic acid moiety is a protein, a carbohydrate, a lipid, a small molecule, or a complex thereof. In any of the embodiments herein, the nucleic acid probe can directly or indirectly bind to a reporter oligonucleotide conjugated to an analyte-binding moiety that directly or indirectly binds to the analyte. In any of the embodiments herein, the analyte-binding moiety can comprise an antibody or epitope binding fragment thereof or an aptamer.

In any of the embodiments herein, the fixed biological sample that is de-crosslinked or additionally fixed and/or the fixed biological sample that is contacted with the nucleic acid probe is the same sample (or a portion thereof) as the fixed biological sample stained with the nucleic acid stain and/or the actin stain. In some embodiments, the fixed biological sample contacted with the nucleic acid probe, without or without having been de-crosslinked or additionally fixed, is the same tissue section as the fixed tissue section stained with the nucleic acid stain and/or the actin stain. In some embodiments, the fixed biological sample contacted with the nucleic acid probe, without or without having been de-crosslinked or additionally fixed, comprises dissociated cells or nuclei from the fixed biological sample stained with the nucleic acid stain and/or the actin stain. In some embodiments, a portion of the fixed biological sample is stained with the nucleic acid stain and/or the actin stain, and the fixed biological sample contacted with the nucleic acid probe, without or without having been de-crosslinked or additionally fixed, comprises dissociated cells or nuclei from the same portion (or a sub-portion thereof) stained with the nucleic acid stain and/or the actin stain.

In any of the embodiments herein, the fixed biological sample that is de-crosslinked or additionally fixed and/or the fixed biological sample that is contacted with the nucleic acid probe and the fixed biological sample stained with the nucleic acid stain and/or the actin stain are different portions of the same biological sample. In some embodiments, the fixed biological sample that is de-crosslinked or additionally fixed and/or the fixed biological sample that is contacted with the nucleic acid probe and the fixed biological sample stained with the nucleic acid stain and/or the actin stain are consecutive sections of the same tissue sample. In some embodiments, a portion of the fixed biological sample is stained with the nucleic acid stain and/or the actin stain, and the fixed biological sample contacted with the nucleic acid probe, without or without having been de-crosslinked or additionally fixed, comprises dissociated cells or nuclei from a portion that is different from the portion stained with the nucleic acid stain and/or the actin stain.

In some embodiments, provided herein is a method for sample analysis, comprising: contacting a fixed tissue section with a nucleic acid stain and/or an actin stain; detecting an optical signal associated with the nucleic acid stain and/or an optical signal associated with the actin stain in the fixed tissue section; comparing the detected optical signal(s) to a reference; contacting the fixed tissue section or a consecutive tissue section thereto in a fixed tissue sample with a nucleic acid probe that directly or indirectly binds to an analyte or a product thereof in the fixed tissue section or consecutive tissue section; and detecting an optical signal associated with the nucleic acid probe or a product thereof at one or more locations in the fixed tissue section or consecutive tissue section, thereby detecting the analyte at the one or locations. In some embodiments, the fixed tissue sample is neither over-fixed nor under-fixed based on the comparing, and the method does not comprise de-crosslinking or additionally fixing the fixed tissue sample prior to the contacting with the nucleic acid probe. In some embodiments, the fixed tissue sample is over-fixed based on the comparing, and the method comprises: de-crosslinking the fixed tissue section or consecutive tissue section; contacting the de-crosslinked fixed tissue section or consecutive tissue section with the nucleic acid probe; and detecting the optical signal in the de-crosslinked fixed tissue section or consecutive tissue section. In some embodiments, the fixed tissue sample is under-fixed based on the comparing, and the method comprises: additionally fixing the fixed tissue section or consecutive tissue section; contacting the additionally fixed tissue section or consecutive tissue section with the nucleic acid probe; and detecting the optical signal in the additionally fixed tissue section or consecutive tissue section.

In any of the embodiments herein, the method can comprise: contacting the fixed tissue section or consecutive tissue section with a first nucleic acid probe that directly or indirectly binds to a first analyte at a first location, and detecting a first optical signal associated with the first nucleic acid probe or a product thereof; and contacting the fixed tissue section or consecutive tissue section with a second nucleic acid probe that directly or indirectly binds to a second analyte at a second location, and detecting a second optical signal associated with the second nucleic acid probe or a product thereof, thereby detecting the first and second analytes at the first and second locations, respectively, in the fixed tissue section or consecutive tissue section. In some embodiments, the first and second analytes are cellular nucleic acid molecules. In some embodiments, the first and second analytes are mRNA molecules of the same gene or different genes. In some embodiments, the first analyte is a cellular nucleic acid molecule, and the second analyte is a protein. In some embodiments, the first analyte is an mRNA molecule and the second analyte is an intracellular protein, a membrane-bound protein, or an extracellular protein. The contacting with the first nucleic acid probe and with the second nucleic acid probe can be simultaneous or in any order, and detecting the first and second optical signal can be simultaneous or in any order. In any of the embodiments herein, the first and second locations can be the same location or different locations.

In any of the embodiments herein, the product of the nucleic acid probe can be generated in situ. In any of the embodiments herein, the optical signal can be detected in situ. In any of the embodiments herein, the optical signal can be detected by imaging the fixed tissue section or consecutive tissue section. In some embodiments, the imaging comprises fluorescent microscopy.

In some embodiments, disclosed herein is a method for sample analysis, comprising: contacting a first portion of a fixed biological sample with a nucleic acid stain and/or an actin stain; detecting an optical signal associated with the nucleic acid stain and/or an optical signal associated with the actin stain in the first portion of the fixed biological sample; comparing the detected optical signal(s) to a reference; contacting dissociated cells or nuclei from the first portion and/or a second portion of the fixed biological sample with a nucleic acid probe that directly or indirectly binds to an analyte or a product thereof in the dissociated cells or nuclei; partitioning the dissociated cells or nuclei in partitions, wherein a single-cell partition comprises one of the dissociated cells or nuclei and a partition barcode; generating a nucleic acid molecule in the single-cell partition, wherein the nucleic acid molecule comprises i) a sequence of the nucleic acid probe or a complement thereof and ii) the partition barcode or a complement thereof, and determining a sequence of the nucleic acid molecule, thereby detecting the analyte in one or more single cells from the fixed biological sample. The partition barcode can be specific to the single-cell partition and uniquely corresponds to the single-cell partition among a plurality of single-cell partitions. In some embodiments, the first and second portions of the fixed biological sample are the same. In some embodiments, the first and second portions of the fixed biological sample are different.

In any of the embodiments herein, the analyte can be an mRNA, and a first nucleic acid probe and a second nucleic acid probe can be hybridized to a first analyte sequence and a second analyte sequence in the mRNA, respectively, wherein: the first nucleic acid probe comprises: i) a first hybridization region complementary to the first analyte sequence, and ii) a first overhang; the second nucleic acid probe comprises: i) a second hybridization region complementary to the second analyte sequence, and ii) a second overhang; and the first and second nucleic acid probes are ligated using the mRNA as template to form a ligated nucleic acid probe, with or without gap filling prior to the ligation. In some embodiments, the single-cell partition is an emulsion droplet or a microwell. In some embodiments, the nucleic acid molecule generated in the single-cell partition comprises the sequences of i) the ligated nucleic acid probe, ii) the partition barcode, and iii) a UMI. In some embodiments, the nucleic acid molecules generated in a plurality of single-cell partitions are sequenced, thereby analyzing analytes in single cells from the fixed biological sample.

In some aspects, provided herein is a method for sample processing and/or analysis, comprising: a) contacting a fixed biological sample with a nucleic acid stain and/or an actin stain; b) detecting an optical signal associated with the nucleic acid stain and/or an optical signal associated with the actin stain in the fixed biological sample; c) comparing the optical signal(s) detected in b) to a reference to determine the quality of the sample; d) transferring an analyte or corresponding probe or ligated probe set from the biological sample to an array of features on a substrate, each of which comprises a spatial barcode sequence associated with a unique spatial location on the array; e) generating a nucleic acid molecule comprises i) the spatial barcode sequence or a complement thereof and ii) a sequence of the analyte or corresponding probe or ligated probe set, or a complement thereof, and f) determining a sequence of the nucleic acid molecule, thereby determining the spatial location of the analyte or corresponding probe or ligated probe set in the biological sample. In some embodiments, the method comprises contacting the biological sample with the probe corresponding to an analyte or with a probe set corresponding to the analyte, wherein the probe or probe set hybridizes to the analyte in the biological sample. In some embodiments, the method comprises contacting the biological sample with a probe set corresponding to the analyte and ligating the probe set to generate the ligated probe set. In some embodiments, the fixed biological sample is de-crosslinked or additionally fixed before contacting with the probe or probe set.

In some aspects, provided herein is a method for sample processing and/or analysis, comprising: a) contacting a fixed biological sample with a nucleic acid stain which selectively binds to RNA over DNA; b) detecting an optical signal associated with the nucleic acid stain in the fixed biological sample; and c) comparing the optical signal detected in b) to a reference to determine the quality of the sample, wherein the comparing optionally comprises using a ratio between the optical signal in the nucleus associated with the nucleic acid stain and an optical signal associated with background signal detected in a cytoplasm, further optionally wherein: (i) where the fixed biological sample is neither over-fixed nor under-fixed based on the comparing in c), the method does not comprise de-crosslinking or additionally fixing the fixed biological sample; (ii) where the fixed biological sample is over-fixed based on the comparing in c), the method comprises de-crosslinking the fixed biological sample to provide a de-crosslinked fixed biological sample; or (iii) where the fixed biological sample is under-fixed based on the comparing in c), the method comprises additionally fixing the fixed biological sample to provide an additionally fixed biological sample.

In some aspects, provided herein is a method for sample processing and/or analysis, comprising: a) contacting a fixed biological sample with a nucleic acid stain which selectively binds to RNA over DNA, optionally wherein the stain is a cyanine dye having the formula:

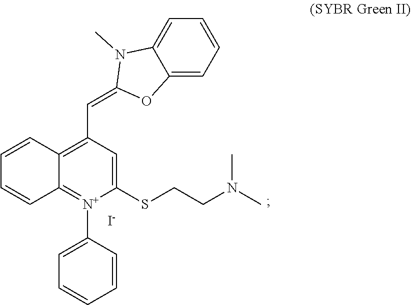 [SMILES: CN(C)CCSc1cc(/C=C2\Oc3ccccc3N2C)c2ccccc2[n+]1-c1ccccc1 ; [I-]]

b) detecting an optical signal associated with the nucleic acid stain in the fixed biological sample; and c) comparing the optical signal detected in b) to a reference to determine the quality of the sample, wherein the comparing optionally comprises using a ratio between the optical signal in the nucleus associated with the nucleic acid stain and an optical signal associated with background signal detected in a cytoplasm, further optionally wherein: (i) where the fixed biological sample is neither over-fixed nor under-fixed based on the comparing in c), the method does not comprise de-crosslinking or additionally fixing the fixed biological sample; (ii) where the fixed biological sample is over-fixed based on the comparing in c), the method comprises de-crosslinking the fixed biological sample to provide a de-crosslinked fixed biological sample; or (iii) where the fixed biological sample is under-fixed based on the comparing in c), the method comprises additionally fixing the fixed biological sample to provide an additionally fixed biological sample.

In some aspects, provided herein is a method for sample processing and/or analysis, comprising: a) contacting a fixed biological sample with a nucleic acid stain which selectively binds to RNA over DNA, optionally wherein the stain is 1-Methyl-4-[(3-methyl-2(3H)-benzothiazolylidene)methyl]quinolinium p-tosylate; b) detecting an optical signal associated with the nucleic acid stain in the fixed biological sample; and c) comparing the optical signal detected in b) to a reference to determine the quality of the sample, wherein the comparing optionally comprises using a ratio between the optical signal in the nucleus associated with the nucleic acid stain and an optical signal associated with background signal detected in a cytoplasm, further optionally wherein: (i) where the fixed biological sample is neither over-fixed nor under-fixed based on the comparing in c), the method does not comprise de-crosslinking or additionally fixing the fixed biological sample; (ii) where the fixed biological sample is over-fixed based on the comparing in c), the method comprises de-crosslinking the fixed biological sample to provide a de-crosslinked fixed biological sample; or (iii) where the fixed biological sample is under-fixed based on the comparing in c), the method comprises additionally fixing the fixed biological sample to provide an additionally fixed biological sample.

In some aspects, provided herein is a method for sample processing and/or analysis, comprising: a) contacting a fixed biological sample with a nucleic acid stain which selectively binds to RNA over DNA, optionally wherein the stain is Thiazole Orange; b) detecting an optical signal associated with the nucleic acid stain in the fixed biological sample; and c) comparing the optical signal detected in b) to a reference to determine the quality of the sample, wherein the comparing optionally comprises using a ratio between the optical signal in the nucleus associated with the nucleic acid stain and an optical signal associated with background signal detected in a cytoplasm, further optionally wherein: (i) where the fixed biological sample is neither over-fixed nor under-fixed based on the comparing in c), the method does not comprise de-crosslinking or additionally fixing the fixed biological sample; (ii) where the fixed biological sample is over-fixed based on the comparing in c), the method comprises de-crosslinking the fixed biological sample to provide a de-crosslinked fixed biological sample; or (iii) where the fixed biological sample is under-fixed based on the comparing in c), the method comprises additionally fixing the fixed biological sample to provide an additionally fixed biological sample.

In some aspects, provided herein is a method for sample processing and/or analysis, comprising: a) contacting a fixed biological sample with a nucleic acid stain which selectively binds to RNA over DNA, optionally wherein the stain is Thiazole Orange Homodimer (TOhD), also known as TOTO®-1; b) detecting an optical signal associated with the nucleic acid stain in the fixed biological sample; and c) comparing the optical signal detected in b) to a reference to determine the quality of the sample, wherein the comparing optionally comprises using a ratio between the optical signal in the nucleus associated with the nucleic acid stain and an optical signal associated with background signal detected in a cytoplasm, further optionally wherein: (i) where the fixed biological sample is neither over-fixed nor under-fixed based on the comparing in c), the method does not comprise de-crosslinking or additionally fixing the fixed biological sample; (ii) where the fixed biological sample is over-fixed based on the comparing in c), the method comprises de-crosslinking the fixed biological sample to provide a de-crosslinked fixed biological sample; or (iii) where the fixed biological sample is under-fixed based on the comparing in c), the method comprises additionally fixing the fixed biological sample to provide an additionally fixed biological sample.

In some aspects, provided herein is a method for sample processing and/or analysis, comprising: a) contacting a fixed biological sample with a nucleic acid stain (such as DAPI) and an actin stain (such as phalloidin or a derivative thereof); b) detecting an optical signal associated with the nucleic acid stain and an optical signal associated with the actin stain in the fixed biological sample; and c) comparing the optical signal(s) detected in b) to a reference to determine the quality of the sample, wherein the comparing optionally comprises using a ratio between the optical signal detected in the cytoplasm associated with the actin stain and an optical signal in the nucleus associated with the nucleic acid stain in the fixed biological sample, further optionally wherein: (i) where the fixed biological sample is neither over-fixed nor under-fixed based on the comparing in c), the method does not comprise de-crosslinking or additionally fixing the fixed biological sample; (ii) where the fixed biological sample is over-fixed based on the comparing in c), the method comprises de-crosslinking the fixed biological sample to provide a de-crosslinked fixed biological sample; or (iii) where the fixed biological sample is under-fixed based on the comparing in c), the method comprises additionally fixing the fixed biological sample to provide an additionally fixed biological sample.

In any of the embodiments herein, the fixed biological sample can be neither over-fixed nor under-fixed based on the comparing, and the fixed biological sample or the dissociated cells or nuclei do not need to be de-crosslinked or additionally fixed prior to contacting with the nucleic acid probe. In any of the embodiments herein, the fixed biological sample can be over-fixed based on the comparing, and the method can comprise: de-crosslinking the fixed biological sample and/or the dissociated cells or nuclei; and contacting the de-crosslinked dissociated cells or nuclei with the nucleic acid probe. The fixed biological sample can be dissociated into single cells or nuclei prior to or after the de-crosslinking. In any of the embodiments herein, the fixed biological sample can be under-fixed based on the comparing, and the method can comprise: additionally fixing the fixed biological sample and/or the dissociated cells or nuclei; and contacting the additionally fixed dissociated cells or nuclei with the nucleic acid probe. The fixed biological sample can be dissociated into single cells or nuclei prior to or after the additional fixing.

In any of the embodiments herein, the fixed biological sample can be a formalin-fixed paraffin-embedded (FFPE) biological sample. In some embodiments, an FFPE tissue section is contacted with a nucleic acid stain and/or an actin stain, and an optical signal associated with the nucleic acid stain and/or an optical signal associated with the actin stain are detected and compared to a reference to assess the level of fixation.

In some embodiments, the FFPE tissue section is over-fixed. The same FFPE tissue section or a portion thereof can be de-crosslinked and dissociated into single cells or nuclei. The de-crosslinking and dissociation can be performed simultaneously or in any order. For instance, the FFPE tissue section or portion thereof can be dissociated into single cells or nuclei, and the single cells or nuclei are then de-crosslinked prior to contacting with the nucleic acid probe. Alternatively, the FFPE tissue section or portion thereof can be de-crosslinked, and the de-crosslinked FFPE tissue section or portion thereof is then dissociated into single cells or nuclei prior to contacting with the nucleic acid probe. In some embodiments, a different FFPE tissue section (the section being different from the FFPE tissue section stained with the nucleic acid stain and/or the actin stain) or a different portion of the same FFPE tissue section (the portion being different from the portion stained with the nucleic acid stain and/or the actin stain) can be de-crosslinked and dissociated into single cells or nuclei. The different FFPE tissue sections can be consecutive sections of the same FFPE tissue (e.g., the same tissue block is consecutively sectioned).

In some embodiments, the FFPE tissue section is under-fixed. The same FFPE tissue section or a portion thereof can be additionally fixed (e.g., crosslinked) and dissociated into single cells or nuclei. The additional fixation and dissociation can be performed simultaneously or in any order. For instance, the FFPE tissue section or portion thereof can be dissociated into single cells or nuclei, and the single cells or nuclei are then additionally fixed prior to contacting with the nucleic acid probe. Alternatively, the FFPE tissue section or portion thereof can be additionally fixed, and the additionally fixed FFPE tissue section or portion thereof is then dissociated into single cells or nuclei prior to contacting with the nucleic acid probe. In some embodiments, a different FFPE tissue section (the section being different from the FFPE tissue section stained with the nucleic acid stain and/or the actin stain) or a different portion of the same FFPE tissue section (the portion being different from the portion stained with the nucleic acid stain and/or the actin stain) can be additionally fixed and dissociated into single cells or nuclei. The different FFPE tissue sections can be consecutive sections of the same FFPE tissue (e.g., the same tissue block is consecutively sectioned).

In any of the embodiments herein, detecting the optical signal associated with the nucleic acid stain and/or the optical signal associated with the actin stain may comprise binning the optical signal detected in each of two or more regions of the biological sample. In any of the embodiments herein, the method may comprise binning a nucleic acid stain result based on the optical signal(s) detected in b) in each of two or more regions of the biological sample, optionally wherein the nucleic acid stain result is a signal-to-noise ratio (SNR) of the optical signal(s), intensity of the optical signal(s), or a Spearman correlation of the optical signal(s) with a DAPI signal.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings illustrate certain features and advantages of this disclosure. These embodiments are not intended to limit the scope of the appended claims in any manner.

FIG. 1A shows representative images of fixed HEK293 cells stained with DAPI, SYTO™ RNASelect™ (“RNASelect” in the figure), and phalloidin. FIG. 1B shows ratios between phalloidin staining and DAPI staining (e.g., “Phalloidin/DAPI intensity ratio” in the figure) that correlate with lengths of the fixation time.

FIG. 2A shows representative images of mouse liver (mLiver) FFPE tissue sections stained with DAPI, RNASelect™, and phalloidin. FIG. 2B shows RNASelect™ nucleus signal-to-noise ratios (SNRs) plotted against the fixation time.

FIG. 3A shows representative images of human lung (hLung) FFPE tissue sections stained with DAPI, RNASelect™, and phalloidin. The tissues were de-paraffinized (“DP”) or de-paraffinized/de-crosslinked (“DLX”). FIG. 3B shows RNASelect™ nucleus/cytoplasm signal-to-noise ratios (SNRs) detected in the DP and D×L samples from two tissue blocks. FIG. 3C shows RCPs detected in samples from the two tissue blocks.

FIG. 4 shows representative images of human brain (hBrain) FFPE tissue sections stained with RNASelect™ and phalloidin, and then used to detect RCPs associated with RNA transcripts of various genes. The anchor sequences in all of the RCPs were also detected.

FIG. 5 shows representative images of human breast (hBreast) FFPE tissue sections stained with RNASelect™, with H&E after RNASelect™ staining, and with H&E staining only.

FIG. 6 shows representative images of human liver FFPE tissue sections stained with two RNA dyes and detected anchor sequences of RCPs associated with RNA transcripts of a panel of six genes.

FIG. 7 shows representative images of human lung and mouse pancreas FFPE tissue sections stained with the indicated dyes.

FIG. 8A nucleus/cytoplasm signal-to-noise ratios (SNRs) for the two tested dyes in two regions of the tissue sample. FIG. 8B shows an example of regional assessment of tissue sample quality.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Biological sample preparation procedures can affect sample quality for downstream analysis. In some cases, various biological samples obtained in the field may vary in quality depending on the source of the sample (e.g., tissue type), the conditions and time from surgery to storage and/or processing of the sample, the amount of time in storage, level of attachment to substrate, and processing of the sample. In some cases, such sample processing procedures are upstream of downstream steps for performing an assay to detect analytes in the sample. Variability in sample preparation procedures and how it affects sample quality can be difficult to predict. For example, a biological sample may be treated using various protocols to preserve analytes in the sample and stored in various conditions prior to being used for an assay to detect analytes in the sample. There may be various reasons for the quality of the analytes in a sample to be sub-optimal. For example, the sample may be stored in a media or solution before fixation and long durations in the storage conditions may degrade RNA quality. In some cases, improper storage at a suboptimal temperature, the sample drying out or exposure to UV light or other environmental factors to a prolonged period of time may degrade tissue and/or analyte quality. In some cases, the pH of the fixative, the size of the sample, and the ratio of sample:fixative may affect sample quality leading to over or under fixation. In some cases, various steps in the tissue processing protocol can be performed for a sub-optimal duration, performed at sub-optimal temperatures, or the reagents such paraffin may be of a sub-optimal quality, and any of such sub-optimal conditions in performing the tissue processing protocol can result in degradation of tissue quality.

For example, fixation is one common method for preservation and storage of biological samples including primary tissue samples for pathology and life science research due to its robustness in maintaining tissue architecture at even ambient temperatures. In particular, formalin/formaldehyde fixation methods are widely used. Suitable fixation allows for maintaining clear and consistent morphologic features for histologic examination. However, either under-fixation and excessive fixation (over-fixation) of samples (e.g., tissue blocks or tissue sections) in various fixatives can lead to tissue architectural changes which can affect its value in downstream analysis. For instance, under-fixed samples may not be able to maintain clear and consistent morphologic features and/or stability of analytes for subsequent analysis. On the other hand, over-fixation due to prolonged and/or improper fixation may result in crosslinking of proteins in the tissue sample and/or a reduction in protein solubility, which can mask target antigens for subsequent detection. In particular, the fixation time of formalin-fixed, paraffin-embedded (FFPE) tissues can be variable and over-fixation can cause decreased RNA signal for in situ detection. While there are guidelines for fixative penetration at 1 mm/hr, obtaining properly fixed samples remains challenging, especially for larger tissues since the outside of a tissue block can be over-fixed whereas the inside of the tissue block can be under-fixed. For smaller tissues, leaving the sample in a fixative (e.g., neutral buffered formalin (NBF), paraformaldehyde (PFA), etc.) over time (e.g., for slightly longer than guidelines) can easily cause over-fixation.

Methods to qualitatively detect the quality of a biological sample, including for example, over-fixation and/or under-fixation of biological samples (such as FFPE, fresh frozen (FF), and cells), are needed. The ability to detect the quality of a sample (e.g., over-fixation and/or under-fixation) would allow for optimization of tissue preparation, including fixation time and/or downstream processing (e.g., increasing de-crosslinking of FFPE samples), as well as subsequent analyte detection. In some embodiments, optimization of tissue preparation, including fixation time and/or downstream processing, can be for the sample assessed using a method disclosed herein (e.g., using a nucleic acid stain and/or an actin stain) and/or for another sample that has not been processed (e.g., a consecutive tissue section to be processed, based on the assessment of quality of the processed tissue section). In some cases, the ability to detect the quality of a sample without performing an analyte detection assay can save time, reagents, and costs if the sample may not have satisfactory performance in the assay.

In some embodiments, provided herein are methods and compositions for detection of signals for assessing the quality of the sample, such as the level of fixation, which can be used to inform downstream sample processing and/or analyte detection. In some cases, the quality of nucleic acid analytes, such as RNA, can be detected. While over-fixation can be suspected when an analyte detection assay on a fixed biological sample show low or no signal, the analyte detection assay may take a long time and the sample can be wasted (e.g., not suitable for repeating the analyte detection assay or for other assays), and the observation of low or no signal per se is not concrete evidence of over-fixation.

In some embodiments, prior to an analyte detection assay, a binder such as an antibody and a dye (e.g., phalloidin, SYTO™ RNASelect™, RNA IQ (e.g., Qubit™ RNA IQ), SYBR™, and/or DAPI) may be used to detect the quality of the biological sample (e.g., under/over-fixation in biological samples) such as FFPE, FF, and cell samples. In some embodiments, the staining and detection can be completed within hours to assess if the sample is optimal for downstream workflows such as in situ detection (e.g., FISH, in situ sequencing, or sequential hybridization of detectable probes), protein staining, spatial array analysis and single cell analysis. In some aspects, assessing the quality of the biological sample (e.g., under/over-fixation can be detected in a fixed biological sample) without having to complete a downstream analyte detection workflow, which may take days to complete and may not provide concrete evidence that the sample is indeed suited for downstream analysis. In some aspects, assessing the quality of the biological sample (e.g., level of fixation) prior to and without downstream analyte detection can also help avoid wasting valuable biological samples.

In some embodiments, a method disclosed herein comprises using a nucleic acid stain (e.g., an RNA stain) and/or an actin stain (e.g., phalloidin or an antibody that specifically recognizes F-actin) for quality control of the fixation of fixed samples and/or optimization of analyte detection in these samples (e.g., by de-crosslinking or additionally fixing the samples). In some embodiments, provided herein is a method comprising: contacting a biological sample with a nucleic acid stain and/or an actin stain, wherein the biological sample is fixed; detecting an optical signal associated with the nucleic acid stain and/or an optical signal associated with the actin stain in the biological sample; and comparing the detected optical signal(s) to a reference. For instance, a nucleic acid stain that selectively stains RNA (e.g., SYTO™ RNASelect™ which is non-fluorescent in absence of nucleic acids) shows a more intense and specific (e.g., indicative that cellular retention of analyte of interest is maintained) signal in samples that are of good quality (e.g., not over-fixed) compared to poor quality (e.g., over-fixed) samples, which have decreased amounts of accessible RNA. In addition, phalloidin binds F-actin and can be detected in samples (e.g., FFPE tissues) with increasing intensity over the time of fixation. As such, a ratio between the phalloidin intensity and DAPI intensity can be used as a reference or normalization: the phalloidin/DPAI ratio increases when the sample is over-fixed and decreases below 1 when the sample is under-fixed. In some aspects, optical signals associated with the nucleic acid stain can be strongly localized in the nucleus when the sample (e.g., cells and FFPE tissue) is under-fixed, and not localized in the nucleus when the sample is over-fixed. Using the actin stain (such as phalloidin) and the nucleic acid stain (e.g., SYTO™ RNASelect™) either separately or in combination can allow for detection of under/over-fixation and optimal fixation in biological samples. The method can be used in any imaging platform for staining and imaging samples, and a ratio between dyes can be used to determine if a sample is over-fixed or under-fixed. Analysis software can be used to quantify the intensity of the dyes, as well as the intensity of other stains such as DAPI.

In some embodiments, based on the actin stain and/or the nucleic acid stain, the method can further comprise adjusting the sample preparation conditions or processing (e.g., level of fixation), and the adjustment can include de-crosslinking or additionally fixing the biological sample. In some embodiments, no adjustment of the level of fixation is necessary based on the quality assessment (e.g., staining). In some embodiments, the method can further comprise contacting the biological sample (e.g., the sample can be de-crosslinked, additionally fixed, or without adjusting the level of fixation) with one or more nucleic acid probes for subsequent analysis, such as in situ analyte detection, spatial array analysis or single cell analysis. In some embodiments, the quality assessment can be used to inform changes in a biological sample preparation protocol to improve the sample quality of other biological samples (e.g., consecutive tissue sections in a fixed tissue block).

### II. Fixed Biological Samples

A biological sample disclosed herein can include any sample comprising a cell, a tissue, or a derivative of a cell or a tissue. In some embodiments, a biological sample herein has been treated to preserve analytes in the sample prior to analysis. In some embodiments, a biological sample herein includes a fixed cell or tissue sample comprising molecular crosslinks that can be catalytically de-crosslinked using a catalyst (e.g., as described in Section VI(B)) disclosed herein. The ability to use a fixed biological sample in an analytical method, such as in situ analysis of biological molecules (e.g., genomic DNA, RNA, cDNA, and/or proteins), can be enhanced in some cases if the cross-links established during fixation of the biological sample are reversed so that an assay can be carried out before sample degradation occurs. In some aspects, data obtained from a de-crosslinked biological sample are similar to that obtained from a fresh sample (e.g., a sample that is not fixed and/or crosslinked).

A fixed biological sample can be any appropriate fixed or otherwise preserved biological sample, including fixed samples obtained by fixing any sample disclosed herein, for instance, in Section VIII. In some embodiments, a fixed biological sample can be a fixed tissue sample (e.g., a fixed tissue section). In some embodiments, a sample herein is not and does not comprise a dissociated tissue/cell suspension. Molecules (e.g., analytes, labelling agents, nucleic acid probes, etc., or products generated in situ in the sample) may but do not need to be removed from a sample herein for analysis before, during, or after catalytic de-crosslinking of the sample. In some embodiments, molecules (e.g., analytes, labelling agents, nucleic acid probes, etc.) are not removed from a sample herein for analysis. In some embodiments, signals associated with the molecules (e.g., analytes, labelling agents, nucleic acid probes, etc., or products generated in situ in the sample) are detected at multiple locations in the catalytically de-crosslinked sample, e.g., the signals can be detected in situ in a catalytically de-crosslinked tissue section.

In some embodiments, the biological sample is fixed and the fixation comprises contacting the sample with one or more agents that react with one another and/or with molecules in the biological sample. In some embodiments, the reaction creates molecular crosslinks between molecules of the one or more agents, between molecules in the biological sample, and/or between molecules of the one or more agents and molecules in the biological sample. In some embodiments, the one or more agents are crosslinking agents, and the molecular crosslinks are products of one or more reactions between a crosslinking agent and a molecule in the biological sample.

In some embodiments, a biological sample is fixed using one or more crosslinking agents comprising an aldehyde. In some embodiments, an aldehyde includes a compound containing one or more aldehyde (—CHO) groups, where the aldehyde groups are capable of reacting with an amine (e.g., a primary amine, a secondary amine, or a tertiary amine) or with an amide. Amines are derivatives of ammonia, wherein one or more hydrogen atoms in amines have been replaced by a substituent such as an alkyl or aryl group. These may respectively be called alkylamines and arylamines, and amines in which both types of substituent are attached to one nitrogen atom may be called alkylarylamines. Exemplary amines include amino acids (including amino acid residues of a protein having side chains that can react with an aldehyde), biogenic amines, trimethylamine, and aniline. In some embodiments, molecular crosslinks in a fixed sample are formed via condensation between an aldehyde and an amine, and in some aspects, the condensation does not require heating and/or an acidic condition. Amides having the structure R—CO—NR′R″ in which a nitrogen atom is attached to a carbonyl group. In some embodiments, molecular crosslinks in a fixed sample are formed via condensation between an aldehyde and an amide, e.g., under heating and/or acidic conditions. Exemplary aldehydes can include formaldehyde, paraformaldehyde, glutaraldehyde, glyoxal, and the like.

In some embodiments, the fixed biological sample is fixed using glyoxal or a derivative thereof. In some embodiments, the fixed biological sample is fixed using bis(sulfosuccinimidyl)suberate or a derivative thereof, e.g., BS³ (Sulfo-DSS).

In some embodiments, fixing a biological sample comprises treating the sample with a crosslinking agent. In some embodiments, the crosslinking agent comprises formaldehyde. Paraformaldehyde (PFA) is a polymer of formaldehyde. While paraformaldehyde itself is not a fixing agent, it can be heated and/or treated under basic conditions until it becomes solubilized and broken down to formaldehyde molecules.

In some embodiments, the molecular crosslinks are on RNA, DNA, protein, carbohydrate, lipid, and/or other molecules in the biological sample. In some embodiments, the molecular crosslinks comprise one or more aminal crosslinks such as aminal bridges. In some embodiments, a fixed biological sample can comprise aminal crosslinks among nucleic acids (e.g., genomic DNA, RNA such as mRNA, and/or cDNA), proteins, carbohydrates, lipids, and/or other molecules in the biological sample. Aminal crosslinks can be made, for example, by fixing a sample with formaldehyde.

In some embodiments, the method comprises fixing the biological sample using a fixative, wherein the fixative comprises 0.01-100% of a fixative selected from the group consisting of: formaldehyde, glutaraldehyde, acetone, methanol, ethanol, acetic acid, potassium dichromate, chromic acid, potassium permanganate, B-5, Zenker's fixative, uranyl acetate, mercuric chloride, osmium tetroxide, potassium permanganate, and 1-ethyl-3-(3-dimethylamino propyl) carbodiimide (EDC), picric acid, and derivatives thereof. In some embodiments, the fixative is free or substantially free of an alcohol. In some embodiments, the fixative is free or substantially free of methanol and ethanol.

In some embodiments, the fixative or fixation agent is formaldehyde. Formaldehyde as fixative comprises paraformaldehyde (or “PFA”) and formalin, both of which relate to the formaldehyde composition (e.g., formalin is a mixture of formaldehyde and methanol). Thus, a formaldehyde-fixed biological sample may be formalin-fixed or PFA-fixed. In some embodiments, the fixative comprises a neutral buffered formalin (NBF) or a paraformaldehyde (PFA) solution. Any suitable protocols and methods for the use of formaldehyde as a fixation reagent to prepare fixed biological samples can be used in the methods and compositions of the present disclosure. In some embodiments, a biological sample is a formalin-fixed, paraffin-embedded (FFPE) tissue sample (e.g., an FFPE tissue section). In some embodiments, the fixed biological sample is de-paraffinized prior to the contacting in a), optionally wherein the de-paraffinizing comprises contacting the biological sample with xylene, ethanol, and water, or, sequentially contacting the biological sample with xylene, absolute ethanol, about 96% ethanol, about 70% ethanol, and water.

In some embodiments, aldehyde fixation methods can be combined with other tissue preservation methods. For example, aldehyde fixation can be combined with fresh frozen preservation of tissues, e.g., fresh frozen tissues can be fixed using an aldehyde. Aldehyde fixation can also be combined with alcohol fixation, or with any number of commercially available fixation/preservation techniques. For example, aldehyde fixation can be combined with salt-rich buffer solutions such as RNAlater™, low-temperature preservation buffers such as HypoThermosol, alcohol-PEG fixation (e.g., Neo-Rix, STATFIX, PAGA, UMFIX), PAXGene, Allprotect/Xprotect, CellCover, RN Assist, and/or zinc buffers.

In some embodiments, preparing fixed (e.g., aldehyde-fixed) biological samples for in situ analysis may comprise catalytic de-crosslinking disclosed herein in combination with additional sample processing steps and/or conditions before, during, and/or after catalytic de-crosslinking. Exemplary sample processing steps and/or conditions may include longer permeabilization periods, additional permeabilization reagents, or higher permeabilization reagent concentrations, e.g., compared to samples that are not fixed, in order to allow detection reagents (e.g., nucleic acid probes and/or antibodies or epitope-binding fragments thereof) to bind to analytes in the sample.

In some embodiments, provided herein are methods of de-crosslinking aminal crosslinks in a fixed biological sample. In some embodiments, provided herein are methods of in situ analysis using such a de-crosslinked sample. The methods described herein are not limited to any particular fixation reagent that results in crosslinks (e.g., aminal crosslinks) and are equally amenable with any fixation method that results in intra-tissue crosslinking events (e.g., aminal intra tissue crosslinking events).

In some embodiments, condensation of an amino group on a first molecule (e.g., nucleic acid or protein) in a sample with formaldehyde can afford a reactive imine, which can react with a proximal amine (e.g., a CH₂-linked amine on a second molecule of the same or different species as the first molecule) to form an aminal bridge, thereby fixing the sample. While fixing can help stabilize the sample, molecular crosslinks could lead to antigen masking and/or background autofluorescence in the sample. In some cases, molecular crosslinks may block or restrict biochemical reactions such as nucleic acid hybridization or methods of signal amplification utilized for analyte detection. Conventional methods for antigen retrieval may not sufficiently retrieve the masked antigens and may not remove or reduce the background autofluorescence due to fixing. The catalytic de-crosslinking methods disclosed herein address these and other issues with conventional methods.

In some embodiments, provided herein are methods for assessing the level of fixation in a biological sample. In some embodiments, the method comprises contacting a fixed biological sample with a nucleic acid stain and/or an actin stain, detecting an optical signal associated with the nucleic acid stain and/or an optical signal associated with the actin stain in the biological sample and assessing the level of fixation of the biological sample based on the optical signal(s) detected in the fixed biological sample. By comparing the optical signal(s) to a reference, sample over-fixation or under-fixation can be detected without having to go through analyte detection in the sample.

### III. Nucleic Acid Staining

Sample preparation treatment procedures and conditions (e.g., fixation times) can be highly variable depending on the type and thickness of the biological sample. In some cases, poor quality samples due to degradation or other conditions e.g., over fixation, can cause decreased signals for in situ, spatial array, or single cell detection of nucleic acid (e.g., RNA). There are currently no concrete methods for the qualitative detection of the quality of the biological sample, e.g., over-fixed or under-fixed biological samples, particularly for the purpose of downstream nucleic acid (e.g., RNA) detection. Thus, a method for detecting the quality of the biological sample, e.g., over-fixation or under-fixation of biological samples, is desired to enable users to optimize fixation time for downstream workflows, including in situ detection and single cell analysis. In some embodiments, provided herein are methods for staining nucleic acid(s) in a fixed biological sample using a nucleic acid stain.

In some embodiments, the nucleic acid stain is a cyanine dye. In some embodiments, the nucleic acid stain is Thiazole Orange, a cyanine dye, which described in U.S. Pat. No. 4,883,867, the content of which is herein incorporated by reference in its entirety, a cyanine dye. In some embodiments, the nucleic acid stain is a Thiazole Orange analog, such as any of the dyes described in EP1,668,162; U.S. Pat. Nos. 5,321,130 and 5,410,030; 7,776,529; and US Appl. 2010/0041045, the contents of each of which are herein incorporated by reference in their entireties. In some embodiments, nucleic acid stain is a cyanine dye comprising a substituted methine bridge and a quinolinium moiety, such as any of the cyanine dyes described in EP1720945, the content of which is herein incorporated by reference in its entirety.

In some embodiments, the nucleic acid dye is a dye that forms a fluorescent complex in combination with double stranded nucleic acids, such as any of the dyes described in U.S. Pat. Nos. 8,598,198 and 9,206,474, the contents of each of which are herein incorporated by reference in their entireties.

In some embodiments, the nucleic acid stain is a light-emitting dye such as EvaGreen dye, a Hoechst dye, SYBR Green I, BEBO, BOXTO, SYTO9, LC Green Plus, ResoLight or Chromofy. In some embodiments, the light-emitting dye is used in conjunction with a light-emitting dye modifier, such as Coomassie Brilliant Blue R-250, Coomassie Brilliant Blue V-250, Coomassie Brilliant Blue G-250, or Guinea Green B. Where desired, the light-emitting dye modifier is designed to be non-fluorescent or not complexed to a metal. In some instances, the light-emitting dye modifier can be a fluorescent dye and exhibits an absorption maximum wavelength of at least about 10 nm longer or shorter than an absorption maximum wavelength of the light-emitting dye. Examples of light-emitting dyes and corresponding light-emitting dye modifiers are described in U.S. Pat. No. 8,148,515, the content of which is herein incorporated by reference in its entirety.

In some embodiments, the nucleic acid stain is a cyanine dye comprising a cyanine structure that connects two fused heterocycle ring systems, where at least two hydrogen-bonding groups are connected to the core structure through linkers. A hydrogen bond is a primarily electrostatic interaction between a hydrogen (H) atom which is covalently bound to a electronegative atom (e.g., O or N) or group of one hydrogen-bonding group, i.e., the hydrogen bond donor, and another electronegative atom having a lone pair of electrons of another hydrogen-bonding group, i.e., the hydrogen bond acceptor. In some embodiments, the hydrogen-bonding group is a moiety or group that includes a H atom bonded to such an electronegative atom (e.g., O or N). In some embodiments, the hydrogen-bonding group is a moiety or group that includes an electronegative atom that is capable of acting as only a hydrogen bond acceptor (e.g., the oxygen atom of a carbonyl containing group C═O). In some embodiments, the hydrogen-bonding group is a moiety that includes a group selected from OH, amino (e.g., a primary amino —NH₂ or a secondary amino group), an amide (e.g., —CONH₂), a urea (e.g., —NHCONH₂), thiourea (e.g., —NHCSNH₂), a sulfonamide group (e.g., —SO₂NH₂), sulfonamide (e.g., —SO₂NH₂), —NHSO₂CH₃, —NHSO₂CH₂F, —NHSO₂CHF₂, and —NHSO₂CF₃. Examples of suitable cyanine dyes are described in US20220260464, the content of which is herein incorporated by reference in its entirety.

In some embodiments, the nucleic acid stain possesses a high membrane permeability. In some embodiments, the nucleic acid stain is cell-permeant and is capable of passing through cell membranes and entering nuclei of cells in a fixed biological sample. In some embodiments, the nucleic acid stain has a membrane permeability that is at least or about 50%, at least or about 75%, at least or about 100%, at least or about 1.5 times, at least or about 2 times, at least or about 5 times, at least or about 7.5 times, at least or about 10 times, at least or about 15 times, at least or about 20 times, or at least or about 25 times of the membrane permeability of a SYTO™ RNASelect™ stain. In some embodiments, the nucleic acid stain is a cell-permeant nucleic acid stain that selectively stains RNA.

In some embodiments, the nucleic acid stain is nonfluorescent or substantially nonfluorescent in the absence of nucleic acids. In some embodiments, the nucleic acid stain is nonfluorescent or substantially nonfluorescent in the absence of nucleic acids. In some embodiments, the nucleic acid stain is fluorescent when bound to RNA. In some embodiments, the nucleic acid stain selectively binds to RNA over DNA.

In some embodiments, a method disclosed herein comprises contacting a fixed sample with one or more nucleic acid stains. In some embodiments, the nucleic acid stain may comprise one or more stain components, for instance, multiple dyes that each stains a different type of nucleic acid or binds to a different structure and/or sequence of the same or different types of nucleic acid. In some embodiments, the nucleic acid stain comprises a dye that binds to intact RNA and/or a dye that binds to degraded RNA. In some embodiments, the intact RNA comprises mRNA, nucleolar RNA, tRNA, and/or rRNA. In some embodiments, the nucleic acid stain comprises a SYTO™ RNASelect™ stain, a RNA integrity and quality (IQ) dye (e.g., Qubit™ RNA IQ kit), a SYTO™ 647 stain, a SYTO™ 17 stain, or a SYTO™ 63 stain. In some embodiments, the nucleic acid stain comprises a StrandBrite™ RNA stain (e.g., as part of the Cell Navigator® RNA imaging kit), such as StrandBrite™ RNA Green. In some embodiments, the nucleic acid stain comprises a QuantiFluor® RNA dye (e.g., as part of the QuantiFluor® RNA system). In some embodiments, the nucleic acid stain comprises a SYBR™ Green II dye.

In some embodiments, the nucleic acid stain is detectable using a detection channel between about 450 nm and about 750 nm in wavelength. In some embodiments, the nucleic acid stain is detectable using a detection channel between about 475 nm and about 700 nm in wavelength. In some embodiments, the nucleic acid stain is detectable using a detection channel between about 480 nm and about 495 nm in wavelength. In some embodiments, the nucleic acid stain is detectable using a detection channel between about 525 nm and about 535 nm in wavelength. In some embodiments, the nucleic acid stain is detectable using a detection channel between about 645 nm and about 655 nm in wavelength. In some embodiments, the nucleic acid stain is detected at a wavelength of about 647 nm. In some embodiments, the nucleic acid stain is detected at a wavelength of about 532 nm. In some embodiments, the nucleic acid stain is detected at a wavelength of about 488 nm.

In some embodiments, the nucleic acid stain comprises DAPI, propidium iodide (PI), a Hoechst stain (e.g., Hoechst 33342), ethidium bromide, a SYBR™ stain (e.g., SYBR™ Green I, SYBR™ Green IL, or SYBR™ Gold), and/or a fluorescent Nissl stain. In some embodiments, the nucleic acid stain does not comprise DAPI, PI, a Hoechst stain (e.g., Hoechst 33342), ethidium bromide, a SYBR™ stain (e.g., SYBR™ Green I, SYBR™ Green IL, or SYBR™ Gold), Thiazole Orange, TOTO®-1 or a fluorescent Nissl stain. In some embodiments, the nucleic acid stain does not comprise an intercalator. In some embodiments, the nucleic acid stain comprises SYBR™ Green II. In some embodiments, the nucleic acid stain does not comprise ethidium bromide.

In some embodiments, the nucleic acid stain is SYBR™ Green II used at a dilution of at least 1:2000 or 1:4000 in a suitable buffer. In some embodiments, the amount of time the sample is stained can be adjusted based on the concentration of the nucleic acid stain. In some embodiments, the nucleic acid stain is diluted in water or PBS.

In some embodiments, the nucleic acid stain selectively binds to RNA. In some embodiments, the nucleic acid stain selectively binds to nucleolar RNA. In some embodiments, the nucleic acid stain has a higher binding affinity to RNA than to DNA. In some embodiments, the binding affinity to RNA of the nucleic acid stain is at least or about 1.1, at least or about 1.2, at least or about 1.5, at least or about 2, at least or about 2.5, at least or about 5, at least or about 10, at least or about 15, at least or about 20, at least or about 25, at least or about 50, at least or about 75, at least or about 100, or at least or about 1,000 times of the binding affinity of the nucleic acid stain to DNA. In some embodiments, the binding affinity of the nucleic acid stain to RNA is more than 5 times, more than 50 times, more than 100 times, more than 200 times, more than 500 times, more than 1,000 times, more than 2,000 times, or more than 5,000 times of its binding affinity to DNA.

In some embodiments, the nucleic acid stain contacts a solvent for the nucleic acid stain and/or the nucleic acid to be stained. In some embodiments, the nucleic acid stain is sensitive to salt. In some embodiments, the nucleic acid stain is sensitive to a divalent cation. In some embodiments, the nucleic acid stain is sensitive to a detergent, such as an ionic detergent and/or a non-ionic detergent. In some embodiments, the nucleic acid stain is sensitive to an ionic detergent but not to a non-ionic detergent, such that binding of the stain to nucleic acid(s) is affected by the ionic detergent but not affected by the non-ionic detergent. In some embodiments, the nucleic acid stain is sensitive to sodium dodecyl sulfate (SDS). In some embodiments, the nucleic acid stain has contacts in a groove (e.g., a major groove and/or a minor groove) of the nucleic acid (e.g., RNA) that it stains, resulting in an increase of the binding constant of the nucleic acid stain-nucleic acid complex. In some embodiments, the nucleic acid shows base selectivity and is likely to have one or more minor groove contacts with the nucleic acid (e.g., RNA) that it stains.

In some embodiments, the nucleic acid stain is contacted with a fixed biological sample under conditions that permit the formation of cellular nucleic acid-stain complexes. The concentration of the nucleic acid stain molecule and the duration of contact time can vary depending upon the application.

In some embodiments, the nucleic acid stain may be removed from nucleic acid using ethanol precipitation. In some embodiments, the nucleic acid stain is not removed from the nucleic acid using butanol or chloroform extraction. In some embodiments, the nucleic acid stain is not removed from the nucleic acid using reagents and/or conditions that remove ethidium bromide from nucleic acid. In some embodiments, the nucleic acid stain is not removed from the nucleic acid using a nonionic detergent. In some embodiments, the nucleic acid stain is not quenched by BrdU.

In some embodiments, the nucleic acid stain comprises a monomethine cyanine analogue. In some embodiments, the nucleic acid stain comprises an unsymmetrical monomethine cyanine analogue. In some embodiments, the nucleic acid stain comprises a styryl dye. In some embodiments, the nucleic acid stain comprises E36. In some embodiments, the nucleic acid stain comprises PI1. In some embodiments, the nucleic acid stain comprises PI2. In some embodiments, the nucleic acid stain comprises (E)-2-(2-(1H-indol-3-yl)vinyl)-1-methylquinolin-1-ium iodide, (E)-2-(2-(1H-indol-2-yl)vinyl)-1-methyl-4-((2-(piperidin-1-yl)ethyl)amino) quinolin-1-ium iodide, or (E)-2-(2-(1H-indol-3-yl)vinyl)-1-methyl-4-((2-(piperidin-1-yl)ethyl)amino) quinolin-1-ium iodide. In some embodiments, the nucleic acid stain comprises any one or more of:

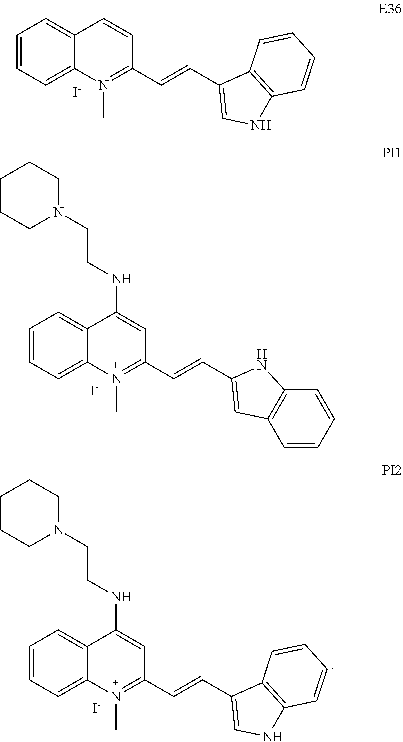

In some embodiments, the nucleic acid stain comprises benzo[c,d]indole-quinoline (BIQ). In some embodiments, the nucleic acid stain comprises a benzo[c,d]indole-containing monomethine cyanine. In some embodiments, the nucleic acid stain comprises benzo[c,d]indole-oxazolopyridine. In some embodiments, the nucleic acid stain comprises a benzo[c,d]indole-oxazolopyridine cyanine dye. In some embodiments, the nucleic acid stain comprises benzo[c,d]indole-oxazolo[5,4-c]pyridine (BIOP) having the formula

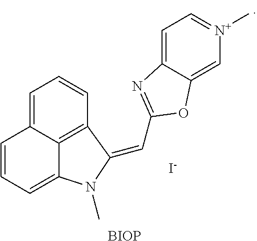

In some embodiments, the nucleic acid stain exhibits a light-up response upon RNA binding, and the light-up factor (I/I₀, where I and I₀ denote the fluorescence intensities in the presence and absence of a target, respectively) is at least or about 100-fold, at least or about 150-fold, at least or about 200-fold, at least or about 250-fold, at least or about 375-fold, at least or about 500-fold, at least or about 550-fold, at least or about 600-fold, at least or about 650-fold, at least or about 700-fold, at least or about 750-fold, at least or about 800-fold, at least or about 850-fold, at least or about 900-fold, at least or about 950-fold, or at least or about 1000-fold. In some embodiments, the I/I₀ of the nucleic acid stain is between about 400-fold and about 1000-fold, for instance, between about 500-fold and about 950-fold, between about 600-fold and about 900-fold, between about 700-fold and about 850-fold, between about 750-fold and about 800-fold, or in a range between any of the aforementioned values.

In some embodiments, the nucleic acid stain exhibits a high fluorescence quantum yield in the bound state with RNA. In some embodiments, the nucleic acid stain has a high Φ_{bound} value. In some embodiments, the nucleic acid stain has a Φ_{bound} value that is at least or about 0.01, at least or about 0.02, at least or about 0.05, at least or about 0.1, at least or about 0.15, at least or about 0.2, at least or about 0.25, at least or about 0.3, at least or about 0.35, at least or about 0.4, at least or about 0.45, at least or about 0.5, at least or about 0.55, at least or about 0.6, at least or about 0.65, at least or about 0.7, at least or about 0.75, or at least or about 0.8. In some embodiments, the Φ_{bound} value of the nucleic acid stain is between about 0.3 and about 0.9, for instance, between about 0.4 and about 0.8, between about 0.45 and about 0.75, between about 0.5 and about 0.7, between about 0.55 and about 0.65, or in a range between any of the aforementioned values.

In some embodiments, the nucleic acid stain exhibits an emission wavelength (λₑₘ) of at least or about 450 nm, at least or about 475 nm, at least or about 500 nm, at least or about 510 nm, at least or about 520 nm, at least or about 530 nm, at least or about 540 nm, at least or about 550 nm, at least or about 560 nm, at least or about 570 nm, at least or about 580 nm, at least or about 590 nm, at least or about 600 nm, at least or about 610 nm, at least or about 620 nm, at least or about 630 nm, at least or about 640 nm, at least or about 650 nm, at least or about 660 nm, at least or about 670 nm, at least or about 680 nm, at least or about 690 nm, or at least or about 700 nm. In some embodiments, the λₑₘ of the nucleic acid stain is between about 480 nm and about 680 nm, for instance, between about 495 nm and about 675 nm, between about 510 nm and about 670 nm, between about 550 nm and about 650 nm, between about 570 nm and about 630 nm, or in a range between any of the aforementioned values.

In some embodiments, the nucleic acid stain has a light-up factor that is at least 100-fold, a Φ_{bound} value that is at least 0.2, and an emission wavelength that is at least 450 nm. In some embodiments, the nucleic acid stain has a light-up factor between about 250-fold and about 950-fold, a Φ_{bound} value between about 0.3 and about 0.8, and an emission wavelength between about 495 nm and about 675 nm. In some embodiments, the nucleic acid stain has a light-up factor that is at least 500-fold, a Φ_{bound} value that is at least 0.4, and an emission wavelength that is at least 550 nm.

The fluorescence of the cellular nucleic acid-stain complexes (e.g., in a fixed cell or tissue sample) may be detected, for example, with an epifluorescence microscope, a confocal microscope, a scanning microscope, a flow cytometer, a fluorometer, and/or a plate reader. For instance, dissociated cells or nuclei stained with the nucleic acid stain can be analyzed using a flow cytometer. In some embodiments, a cell or tissue sample or dissociated cells or nuclei on a substrate (e.g., on a planar substrate or in wells) stained with the nucleic acid stain can be analyzed using microscopy and/or a plate reader. In some aspects, the nucleic acid stain in a biological sample can be detected using fluorescent microscopy or other imaging techniques described herein. In such aspects, the detecting comprises determining a signal, e.g., a fluorescent signal. In some aspects, the detection (comprising imaging) is carried out using any of a number of different types of microscopy, e.g., confocal microscopy, two-photon microscopy, or light-field microscopy.

### IV. Cellular Matrix Staining

In some embodiments, provided herein are methods for staining a fixed biological sample using one or more stains for the cellular matrix in the fixed biological sample. In some embodiments, the cellular matrix comprises the intracellular cytoskeleton, the extracellular matrix, and cell-cell and cell-matrix junctions (e.g., intermediate filaments can form cell-cell connections and anchor cell-matrix junctions).

The cytoskeleton is a complex, dynamic network of interlinking protein filaments present in the cytoplasm of all cells, whereas the extracellular matrix is a large network of proteins and other molecules that surround, support, and give structure to cells and tissues in the body. In eukaryotes, the cytoskeleton extends from the cell nucleus to the cell membrane and is composed of similar proteins in the various organisms, including three main components: microfilaments, intermediate filaments, and microtubules. Microfilaments comprise actin, one of the most abundant cellular proteins. Actin can be present as either a free monomer called G-actin (globular) or as part of a linear polymer microfilament called F-actin (filamentous).

In some embodiments, signals associated with the one or more cellular matrix stains can provide information regarding the fixation (e.g., cross-linking) status of the cellular matrix, thereby providing an indicator of the overall level of fixation (e.g., cross-linking) of the fixed biological sample. In some embodiments, the fixed biological sample is stained with a cellular matrix stain that binds directly or indirectly to one or more components of the extracellular matrix. In some embodiments, the fixed biological sample is stained with a cellular matrix stain that binds directly or indirectly to one or more components of the cytoskeleton. In some embodiments, the fixed biological sample is stained with an actin stain. In some embodiments, the actin stain selectively binds to F-actin over monomeric or oligomeric actin.

In some embodiments, the fixed biological sample is stained with phalloidin or a derivative or analogue thereof. Phalloidin belongs to a class of toxins called phallotoxins, which are found in the death cap mushroom Amanita phalloides. It is a rigid bicyclic heptapeptide that functions by binding and stabilizing F-actin (much more tightly than to actin monomers) and effectively prevents the depolymerization of actin fibers. Due to its tight and selective binding to F-actin, phalloidin and derivatives thereof (e.g., derivatives containing fluorescent tags) can be used to visualize F-actin in a cell or tissue sample, such as a tissue section or dissociated cells. In some aspects, a biological sample with more fixation stained with phalloidin shows more intense staining compared to a biological sample with less fixation for certain tissue types.

In some embodiments, the fixed biological sample is stained with a binder that specifically or selectively binds to F-actin. In some embodiments, the binder is an aptamers or an antibody or epitope binding fragment thereof that binds to F-actin.

The fluorescence of the cellular matrix stain (e.g., phalloidin and a derivative thereof that is used to stain a fixed cell or tissue sample) may be detected, for example, with an epifluorescence microscope, a confocal microscope, a scanning microscope, a flow cytometer, a fluorometer, and/or a plate reader. For instance, dissociated cells or nuclei stained with the cellular matrix stain (e.g., an F-actin stain such as phalloidin or a derivative thereof) can be analyzed using a flow cytometer. In some embodiments, a cell or tissue sample or dissociated cells or nuclei on a substrate (e.g., on a planar substrate or in wells) stained with the cellular matrix stain (e.g., an F-actin stain such as phalloidin or a derivative thereof) can be analyzed using microscopy and/or a plate reader. In some aspects, the cellular matrix stain in a biological sample can be detected using fluorescent microscopy or other imaging techniques described herein. In such aspects, the detecting comprises determining a signal, e.g., a fluorescent signal. In some aspects, the detection (comprising imaging) is carried out using any of a number of different types of microscopy, e.g., confocal microscopy, two-photon microscopy, or light-field microscopy.

### V. Assessing Level of Fixation

In some embodiments, provided herein is a method for sample processing and/or analysis, comprising: contacting a fixed biological sample with a nucleic acid stain and/or an actin stain; detecting an optical signal associated with the nucleic acid stain and/or an optical signal associated with the actin stain in the fixed biological sample; and comparing the detected optical signal(s) to a reference in order to assess the quality (e.g., level of fixation) of the fixed biological sample. In some embodiments, the method comprises using the nucleic acid stain but not the actin stain, and the detected optical signal associated with the nucleic acid stain is compared to a reference in order to assess the level of fixation in the fixed biological sample. In some embodiments, the method comprises using the actin stain but not the nucleic acid stain, and the detected optical signal associated with the actin stain is compared to a reference in order to assess the quality of the fixed biological sample. In some embodiments, the method comprises using the nucleic acid stain and the actin stain, and the detected optical signals associated with the actin stain and the nucleic acid stain are compared to one or more references in order to assess the quality of the fixed biological sample. In some embodiments, the quality of the fixed biological sample is reflective of the level of fixation in the biological sample. In some aspects, the quality of the fixed biological sample comprises the preservation of certain biomolecules and structures in the biological sample, and/or the quality of certain analytes in the biological sample, e.g., RNA analytes.

In some embodiments, a fixed biological sample can be contacted with a first nucleic acid stain and a second nucleic acid stain simultaneously or in any order. In some embodiments, the first nucleic acid stain selectively binds to RNA and the second nucleic acid stain binds to both DNA and RNA or selectively binds to DNA. In some embodiments, the second nucleic acid stain can comprise DAPI, propidium iodide (PI), a Hoechst stain, or a fluorescent Nissl stain. In some embodiments, the optical signal associated with the first nucleic acid stain and the optical signal associated with the second nucleic acid stain can be detected in a nucleus. In some embodiments, the optical signals can be detected in the nuclei of cells in the fixed biological sample. In some embodiments, the comparing can comprise using a ratio between the optical signal associated with the first nucleic acid stain (e.g., SYTO™ RNASelect™) and the optical signal associated with the second nucleic acid stain (e.g., DAPI) in the fixed biological sample, and comparing the ratio to a reference. In some embodiments, the reference is a reference ratio between the optical signal associated with the first nucleic acid stain (e.g., SYTO™ RNASelect™) and the optical signal associated with the second nucleic acid stain (e.g., DAPI) detected in the same fixed sample (e.g., a different region or portion of the same sample), or detected in a reference sample (e.g., a sample with a known or estimated level of fixation), or the reference ratio can be provided based on detection in one or more similar samples (e.g., the reference can be based on detection in multiple mouse brain samples and used for assessing fixation of a mouse brain sample). In some embodiments, the first nucleic acid stain is SYBR™ Green II and the second nucleic acid stain is DAPI. In some embodiments, one or more reference images or samples can be provided for generating a reference ratio.

In some embodiments, the reference ratio is about 1, about 1.5, about 2, about 2.5, about 3, about 3.5, about 4, about 4.5, or about 5. In some embodiments, the reference ratio of a nucleus/cytoplasm SNR can be compared to a detected nucleus/cytoplasm SNR of a biological sample. In some embodiments, a detected ratio between the optical signal associated with the first nucleic acid stain (e.g., SYTO™ RNASelect™) and the optical signal associated with the second nucleic acid stain (e.g., DAPI) or nucleus/cytoplasm SNR ratio more than the reference ratio indicates the biological sample can be used for downstream analyte detection (e.g., described in Section VII). In some embodiments, a detected ratio between the optical signal associated with the first nucleic acid stain (e.g., SYTO™ RNASelect™) and the optical signal associated with the second nucleic acid stain (e.g., DAPI) more than the reference ratio indicates the biological sample is not over-fixed. In some embodiments, a detected ratio more than about 1, about 1.5, about 2, about 2.5, about 3, about 3.5, about 4, about 4.5, or about 5 indicates the biological sample is not over-fixed. In some embodiments, a detected nucleus/cytoplasm SNR ratio greater than about 2 indicates the biological sample is not over-fixed. In some embodiments, a detected nucleus/cytoplasm SNR ratio less than about 1 indicates the biological sample is over-fixed. In some embodiments, a detected ratio between the optical signal associated with the first nucleic acid stain (e.g., SYTO™ RNASelect™) and the optical signal associated with the second nucleic acid stain (e.g., DAPI) or nucleus/cytoplasm SNR ratio of less than the reference ratio indicates the biological sample is over-fixed. In some embodiments, a detected nucleus/cytoplasm SNR ratio greater than about 5, about 5.5, about 6, about 6.5, about 7, about 7.5, about 8, about 8.5, about 9, about 9.5, or about 10 indicates the biological sample is suitable for downstream analyte detection. In some embodiments, the reference nucleus/cytoplasm SNR ratio is about 2 and a detected nucleus/cytoplasm SNR ratio of less than 2 indicates the biological sample is of low quality and not suitable for downstream analyte detection (e.g., described in Section VII). In some embodiments, the stains used for calculating the SNR comprises a first nucleic acid stain that is SYBR™ Green II and a second nucleic acid stain that is DAPI.

In some embodiments, a phalloidin/DAPI intensity ratio less than about 1 can indicate the sample is under-fixed or not over-fixed. In some embodiments, a phalloidin/DAPI intensity ratio over than about 2, about 2.5, about 3, about 3.5, about 4, about 4.5, or about 5 can indicate the sample is of low quality is not suitable for analyte detection. In some embodiments, a phalloidin/DAPI intensity ratio over than about 5 can indicate the sample is of low quality is not suitable for analyte detection, and a phalloidin/DAPI intensity ratio less than about 1 can indicate the sample is under-fixed. In some embodiments, a phalloidin/DAPI intensity ratio between about 1 and about 5 can indicate the sample is of good quality and suitable for downstream analyte detection. In some embodiments, a phalloidin/DAPI intensity ratio between about 1 and about 4 can indicate the sample is of good quality and suitable for downstream analyte detection. In some embodiments, a phalloidin/DAPI intensity ratio between about 1 and about 3 can indicate the sample is of good quality and suitable for downstream analyte detection.

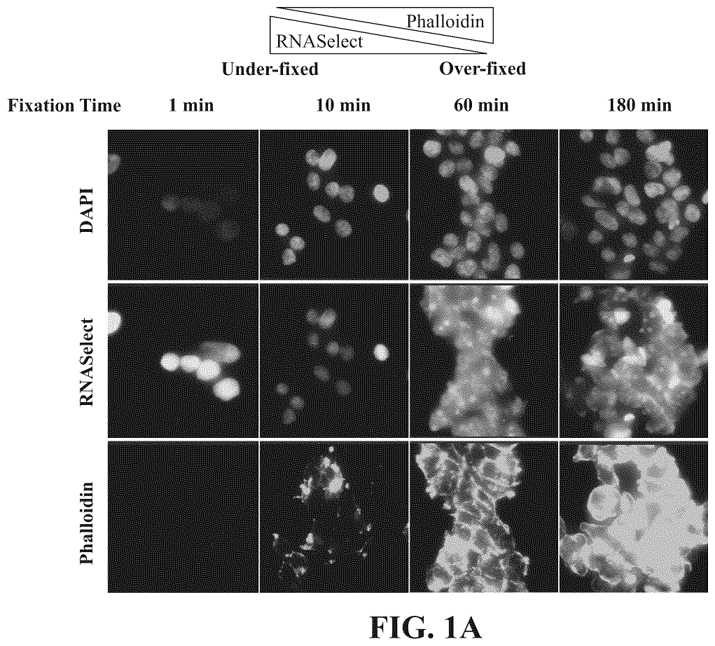

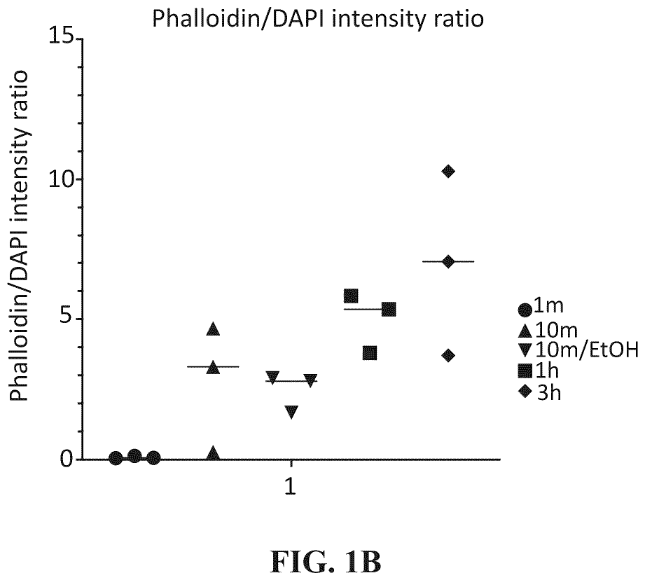

FIGS. 1A-1B show that the ratios between phalloidin staining and DAPI staining (e.g., “Phalloidin/DAPI intensity ratio” in the figure) can be correlated with lengths of the fixation time and the level of sample fixation. In some embodiments, a phalloidin/DAPI intensity ratio less than about 1 can indicate the sample is under-fixed and a phalloidin/DAPI intensity ratio of about 5 or greater can indicate the sample is over-fixed.

In some embodiments, a detected nucleus/cytoplasm SNR ratio greater than about 2, about 3, about 4, about 5, about 5.5, about 6, about 6.5, about 7, about 7.5, about 8, about 8.5, about 9, about 9.5, or about 10, and/or a phalloidin/DAPI intensity ratio less than about 1, about 2, about 3, about 4, or about 5 can indicate the biological sample is suitable for downstream analyte detection. In some embodiments, a detected nucleus/cytoplasm SNR ratio less than about 2, and/or a phalloidin/DAPI intensity ratio greater than about 5 can indicate the biological sample is of low quality (e.g., over-fixed) and not suitable for downstream analyte detection.

In some embodiments, the comparing can comprise using a ratio between the optical signal associated with the actin stain and an optical signal associated with an additional nucleic acid stain in the fixed biological sample. A detected ratio can be compared to a reference ratio detected in the same fixed sample (e.g., a different region or portion of the same sample), or detected in a reference sample (e.g., a sample with a known or estimated level of fixation), or the reference ratio can be provided based on detection in one or more similar samples. In some embodiments, one or more reference images or samples can be provided for generating a reference ratio. In some embodiments, the additional nucleic acid stain selectively binds to DNA. In some embodiments, the additional nucleic acid stain is DAPI. In some embodiments, the optical signal associated with the actin stain can be detected in a cytoplasm and the optical signal associated with the additional nucleic acid stain can be detected in a nucleus.

In some embodiments, the comparing can comprise using an optical signal associated with the nucleic acid stain, an optical signal associated with the actin stain, an optical signal associated with an additional nucleic acid stain, and/or an optical signal associated with an additional actin stain in a reference sample. For example, the comparing can comprise quantitatively and/or qualitatively comparing optical signals in on image of the fixed biological sample to optical signals in one or more other images of the fixed biological sample or a reference sample different from the fixed biological sample. In some embodiments, an elevated level of the nucleic acid stain and/or the additional nucleic acid stain in the fixed biological sample compared to that in the reference sample can indicate the fixed biological sample is less fixed than the reference sample. In some embodiments, a decreased level of the nucleic acid stain and/or the additional nucleic acid stain in the fixed biological sample compared to that in the reference sample can indicate the fixed biological sample is more fixed than the reference sample. In some embodiments, an elevated level of the actin stain and/or the additional actin stain in the fixed biological sample compared to that in the reference sample can indicate the fixed biological sample is more fixed than the reference sample. In some embodiments, a decreased level of the actin stain and/or the additional actin stain in the fixed biological sample compared to that in the reference sample can indicate the fixed biological sample is less fixed than the reference sample.

In some embodiments, the comparing can comprise using a signal-to-noise ratio (SNR) between signals associated with the nucleic acid stain (e.g., an RNA-selective stain) in the nucleus and signals associated with another nucleic acid stain (e.g., a DNA-selective stain). In some embodiments, the comparing can comprise using a signal-to-noise ratio (SNR) between signals associated with an RNA-selective stain in the nucleus and signals associated with the same or a different RNA-selective stain in the cytoplasm. The location of the nucleus and/or the boundary between the nucleus and the cytoplasm can be detected using another nucleic acid stain (e.g., a DNA-selective stain), in order to distinguish signals associated with RNA-selective stain(s) in nuclei from RNA-selective stain signals in the cytoplasm. A detected nucleus/cytoplasm SNR can be compared to a reference nucleus/cytoplasm SNR detected in the same fixed sample (e.g., a different region or portion of the same sample), or detected in a reference sample (e.g., a sample with a known or estimated level of fixation), or the reference nucleus/cytoplasm SNR can be provided based on detection in one or more similar samples. In some embodiments, one or more reference images or samples can be provided for generating a reference nucleus/cytoplasm SNR. SNR generation or a nucleus-to-cytoplasm signal ratio can be done manually or automated (e.g., using a software).

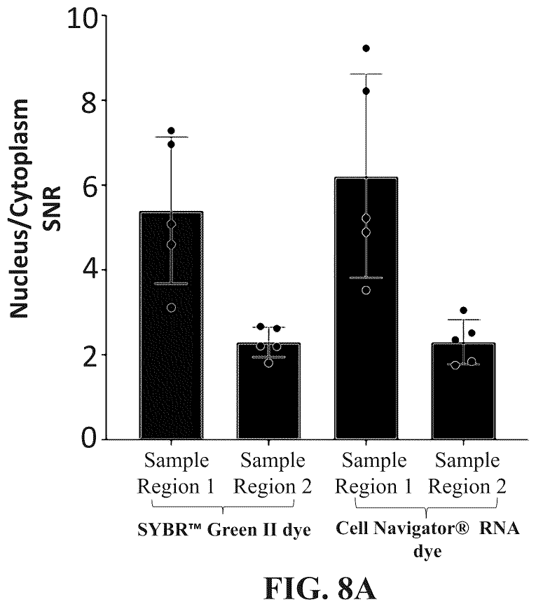

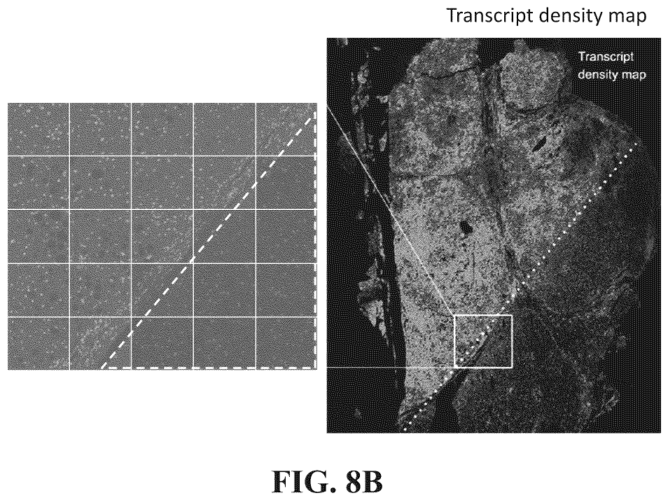

In some embodiments, the analysis can comprise binning nucleic acid stain results (e.g., SNR, detected intensity, or Spearman correlation with DAPI signal) in each of two or more regions of a tissue sample. For example, a tissue sample may be divided in a plurality of regions (e.g., as shown in FIG. 8B). In some embodiments, the SNR can be binned within each region of the tissue sample. In some embodiments, software may be used to automate the processing (e.g., region determination and/or binning), analysis, and/or display of data. Analysis software can be used to quantify staining results (e.g., SNR, detected intensity, or Spearman correlation with DAPI signal) within one or more regions of the tissue sample. Analysis software can be used to compare the staining results (e.g., SNR, detected intensity, or Spearman correlation with DAPI signal) of the nucleic stain between two or more regions of the tissue sample.

In some embodiments, the tissue sample may be divided into regions (e.g., rectangular regions as shown in FIG. 8B) and a percentage of the area in the particular region exhibiting staining indicative of good quality is determined. In some aspects, as the rolling window for binning of the sample decreases, the resolution may increase. In some cases, a display of the tissue sample may provide an indication of whether the region contains areas of good quality or poor quality. For example, a region may be considered to have a satisfactory percentage of good quality cells for downstream analysis if more than 50%, more than 60%, more than 70%, more than 80%, or more than 90% of the binned area shows staining indicative of good quality. In some cases, a region may be considered to not have a satisfactory percentage of good quality cells for downstream analysis if more than 50%, more than 60%, more than 70%, more than 80%, or more than 90% of the binned area shows staining indicative of poor quality.

In some embodiments, the tissue sample may be divided into regions (e.g., rectangular regions as shown in FIG. 8B) and a percentage of cells in the particular region exhibiting staining indicative of good quality is determined. In some cases, a display of the tissue sample may provide an indication of whether the region contains cells of good quality or poor quality. For example, a region may be considered to have a satisfactory percentage of good quality cells for downstream analysis if more than 50%, more than 60%, more than 70%, more than 80%, or more than 90% of cells in the region show staining indicative of good quality. In some cases, a region may be considered to not have a satisfactory percentage of good quality cells for downstream analysis if more than 50%, more than 60%, more than 70%, more than 80%, or more than 90% of cells in the region show staining indicative of poor quality.

In some embodiments, the Spearman correlation between two nucleic acid stains (e.g., a SYBR™ Green II dye and DAPI) can be determined for each region in a tissue sample. In some aspects, intensity of a first nucleic acid stain and a second nucleic acid stain may be correlated in a sample (or a region thereof) of good quality. In some aspects, intensity of a first nucleic acid stain in the nucleus and a second nucleic acid stain in the nucleus may be correlated in a sample (or a region thereof) of good quality.

In some embodiments, it can be useful to determine regional differences in tissue quality for samples because some portions of a tissue may exhibit low quality cells or analytes. In some cases, it can be difficult to determine any regional differences when evaluating the entire tissue sample (e.g., in a zoomed out view) or when determining an average SNR of the entire tissue sample. In some cases, a method to bin the detected signals from one or more stains or a calculated score based on the stain(s) e.g., intensity of stain, SNR scores, or Spearman correlation with DAPI stain, in each region can provide a quality map for the tissue. In some cases, the quality map can be used to select regions of interest, to screen samples prior to placing on a substrate for performing an assay, to screen samples prior to performing an assay on an instrument, and/or to remove data from certain identified regions of low quality during analysis.

In some embodiments, a fixed biological sample can be stained with a nucleic acid stain such as RNASelect™ and/or an actin stain, imaged to assess the level of sample fixation, and then stained for sample morphology, such as with H&E staining. The sample can be optionally de-crosslinked or additionally fixed prior to and/or after staining for morphology (e.g., using H&E staining).

### VI. Adjusting or Controlling Level of Fixation

Depending on the assessment of sample fixation, the fixed biological sample can be de-crosslinked if it is over-fixed, or the fixed biological sample can be additionally fixed if it is under-fixed. In some embodiments, provided herein is a method for sample processing, comprising: a) contacting a biological sample with a nucleic acid stain and/or an actin stain, wherein the biological sample is fixed; b) detecting an optical signal associated with the nucleic acid stain and/or an optical signal associated with the actin stain in the biological sample; c) comparing the optical signal(s) detected in b) to a reference; d) adjusting to a level of fixation in the biological sample by: i) performing additional fixation; or ii) performing de-crosslinking.

In some embodiments, provided herein is a method for sample analysis, comprising: a) contacting a biological sample with i) an RNA stain and ii) an actin stain, wherein the biological sample is fixed, the RNA stain selectively binds to RNA over DNA, and the actin stain is phalloidin or a derivative thereof; b) detecting optical signals associated with the RNA stain and the actin stain in the biological sample; and c) assessing the level of fixation of the biological sample based on the optical signals detected in b). In some embodiments, the method further comprises: d) crosslinking or de-crosslinking the fixed biological sample based on the assessing in c).

In some embodiments, biological samples that have not been fixed can be fixed in an informed way using the assessment of sample fixation. For example, a tissue block can be sectioned into consecutive tissue sections, and a first tissue section of the consecutive sections can be fixed and then stained with a nucleic acid stain and/or an actin stain disclosed herein. The fixation level in the fixed first tissue section can be assessed, and if the first tissue section is over-fixed or under-fixed, a second tissue section consecutive to the first tissue section can be fixed under a different condition to achieve optimal fixation. For instance, if the first tissue section is over-fixed, the second, consecutive tissue section can be fixed for a shorter time and/or using a less strong fixative. Likewise, dissociated single cells can be divided into separate portions, and a first portion can be fixed and then stained with a nucleic acid stain and/or an actin stain disclosed herein. The fixation level in the fixed first portion can be assessed to guide the fixation of another portion of the same population of dissociated single cells.

A. Fixation and Additional Fixation

In some embodiments, a biological sample can be fixed, and a fixed biological sample can be additionally fixed. In some embodiments, a fixed biological sample and/or additionally fixed biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a fixed biological sample and/or additionally fixed biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe (e.g., a padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

B. Crosslinking and De-Crosslinking

In some embodiments, provided herein are methods and compositions for providing a fixed biological sample immobilized on a substrate, and for catalytically de-crosslinking molecular crosslinks in the fixed biological sample. In some embodiments, a method disclosed herein comprises contacting a fixed biological sample with a composition comprising a catalyst or a precursor thereof.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel comprises a hybrid material, e.g., the hydrogel material comprises elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate comprises a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of molecular crosslinks in the reversibly cross-linked biological sample are de-crosslinked.

In some embodiments, a de-crosslinking agent or un-fixing agent herein can be a compound or composition that reverses fixation and/or removes the crosslinks within or between biomolecules (e.g., analytes for analytical methods, such as those described herein) in a sample caused by previous use of a fixation reagent. In some embodiments, de-crosslinking agents are compounds that act catalytically in removing crosslinks in a fixed sample. In some embodiments, de-crosslinking agents are compounds that act catalytically in removing aminal crosslinks in a fixed sample. In some embodiments, de-crosslinking agents can act on biological samples fixed with an aldehyde (e.g., formaldehyde), an N-hydroxysuccinimide (NHS) ester, an imidoester, or a combination thereof.

In some embodiments, provided herein are catalysts that catalyze de-crosslinking of inter-molecular crosslinks and/or intra-molecular crosslinks in the biological sample. In some embodiments, provided herein are catalysts that catalyze the cleavage of aminal bridges, thereby de-crosslinking the inter-molecular crosslinks and/or intra-molecular crosslinks.

In some embodiments, the de-crosslinking provided herein can comprise a protease mediated decrosslinking or proteolytic-induced antigen retrieval (PIER), and the protease can be any suitable protease such as proteinase K, pepsin, or a combination thereof. In some embodiments, the de-crosslinking provided herein can comprise heat-induced antigen retrieval (HIER) using one or more buffers, such as Citrate buffer pH 6.0, Tris buffer pH 8.0, or Tris-EDTA buffer pH 9.0.

In some embodiments, the catalyst is a water-soluble catalyst. In some embodiments, the catalyst is an organic molecule. In some embodiments, the catalyst is a transimination catalyst. In some embodiments, the catalyst is a bifunctional transimination catalyst that accelerates hydrazone and oxime formation. In some embodiments, the catalyst catalyzes de-crosslinking of aminal crosslinks in the biological sample. In some embodiments, the catalyst catalyzes breakdown of hemi-aminal adducts and/or aminal adducts in the biological sample.

Aminal crosslinks (e.g., aminal bridges) can be catalytically reversed using one or more organocatalyst. In some embodiments, in catalytic reversal of aminal crosslinks, a first C—N bond of the aminal bridge can be broken in an acid-base reaction, and the second C—N bond of the aminal can be broken to generate repaired NH₂ groups on the first and second molecules. In some embodiments, aminal crosslinks can be catalytically reversed using a combination of acid catalysis and nucleophilic catalysis.

In some embodiments, the sample is contacted with a compound (e.g., in a solution or suspension) for catalytic de-crosslinking selected from the group consisting of 2-amino-5-methylbenzoic acid, 2-amino-5-nitrobenzoic acid, (2-amino-5-methylphenyl)phosphonic acid, 2-amino-5-methylbenzenesulfonic acid, 2,5-diaminobenzenesulfonic acid, 2-amino-3,5-dimethylbenzenesulfonic acid, (2-amino-5-nitrophenyl)phosphonic acid, (4-aminopyridin-3-yl)phosphonic acid, (3-aminopyridin-2-yl)phosphonic acid, (5-aminopyrimidin-4-yl)phosphonic acid, (2-amino-5-{[2-poly-ethoxy]ethyl}carbamoyl)phenyl)phosphonic acid, 4-aminonicotinic acid, 3-aminoisonicotinic acid, 2-aminonicotinic acid, and (2-aminophenyl)phosphonic acid. In some embodiments, the sample is contacted with Compound 1 (2-amino-5-methylbenzoic acid) in a solution or suspension for catalytic de-crosslinking. In some embodiments, the sample is contacted with Compound 8 ((4-aminopyridin-3-yl)phosphonic acid) in a solution or suspension for catalytic de-crosslinking. In some embodiments, the sample is contacted with Compound 15 ((2-aminophenyl)phosphonic acid) in a solution or suspension for catalytic de-crosslinking.

In some embodiments, the catalyst is selected from the group consisting of (2S,4R)-4-hydroxyproline, (2R,4S)-4-hydroxyproline, (2S,4S)-4-hydroxyproline, (2R,4R)-4-hydroxyproline, (2S,4R)-4-aminoproline, (2R,4S)-4-aminoproline, (2S,4S)-4-aminoproline, and (2R,4R)-4-aminoproline.

In some embodiments, the catalyst comprises

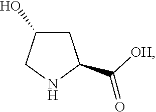

or a salt, zwitterion, or solvate thereof. In some embodiments, the catalyst comprises

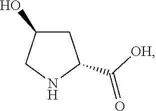

or a salt, zwitterion, or solvate thereof. In some embodiments, the catalyst comprises

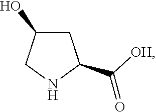

or a salt, zwitterion, or solvate thereof. In some embodiments, the catalyst comprise

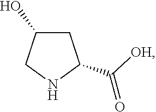

or a salt, zwitterion, or solvate thereof. In some embodiments, the catalyst comprises

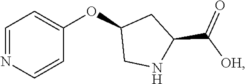 [SMILES: O=C(O)[C@@H]1C[C@H](Oc2ccncc2)CN1]

or a salt, zwitterion, or solvate thereof. In some embodiments, the catalyst comprises

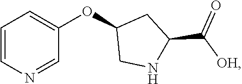 [SMILES: O=C(O)[C@@H]1C[C@H](Oc2cccnc2)CN1]

or a salt, zwitterion, or solvate thereof.

In some embodiments, the catalyst is selected from the group consisting of:

| Compound No. | Compound Structure |
|---|---|
| 1 | 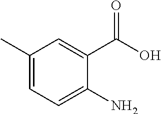 |
| 2 | 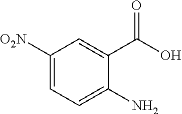 [SMILES: Nc1ccc([N+](=O)[O-])cc1C(=O)O] |
| 3 | 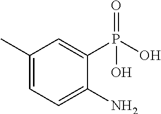 |
| 4 | 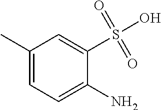 |
| 5 | 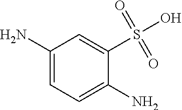 [SMILES: Nc1ccc(N)c(S(=O)(=O)O)c1] |
| 6 | 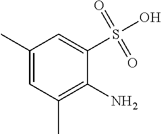 |
| 7 | 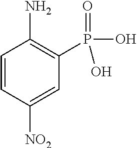 [SMILES: Nc1ccc([N+](=O)[O-])cc1P(=O)(O)O] |
| 8 | 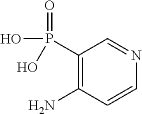 [SMILES: Nc1ccncc1P(=O)(O)O] |
| 9 | 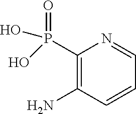 [SMILES: Nc1cccnc1P(=O)(O)O] |
| 10 | 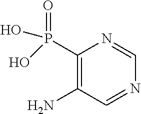 [SMILES: Nc1cncnc1P(=O)(O)O] |
| 11 | 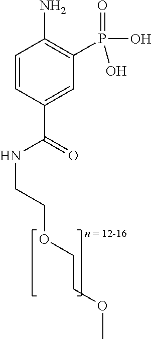 |
| 12 | 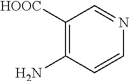 |
| 13 | 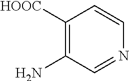 |
| 14 | 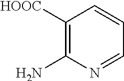 |
| 15 | 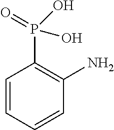 [SMILES: Nc1ccccc1P(=O)(O)O] |
| 16 | 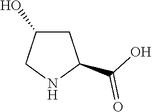 |
| 17 | 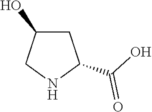 |
| 18 | 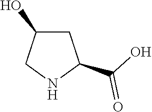 |
| 19 | 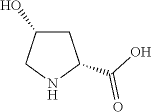 |
| 20 | 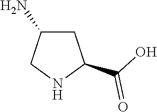 |
| 21 | 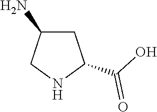 |
| 22 | 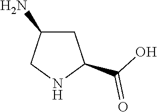 |
| 23 | 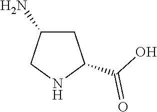 |
| 24 | 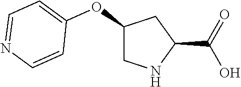 [SMILES: O=C(O)[C@@H]1C[C@H](Oc2ccncc2)CN1] |
| 25 | 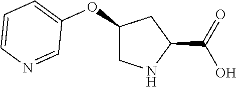 [SMILES: O=C(O)[C@@H]1C[C@H](Oc2cccnc2)CN1] |

or a salt, Zwitterion, or solvate thereof.

A composition comprising a catalyst for de-crosslinking disclosed herein may additionally comprise water, various non-ionic detergents, saline-sodium citrate (SSC), sodium phosphate, phosphate buffered saline (PBS), sodium dodecyl sulfate (SDS), urea, proteinase (e.g., proteinase K), bovine serum albumin (BSA), ethylenediaminetetraacetic acid (EDTA), a sarkosyl compound (e.g., sodium lauroyl sarcosinate; sarkosyl, ammonium salt; or sarkosyl, potassium salt), tris(hydroxymethyl)aminomethane (tris), tris-HCl (tris hydrochloride), 3-morpholinopropane-1-sulfonic acid (MOPS), TAE buffer (tris EDTA), TBS buffer (tris buffered saline), bis-tris methane, 4-(2-hydroxyethyl)-1-piperazineethanesulfonic acid (HEPES buffer), dimethyl sulfoxide (DMSO), quaternary ammonium salts (e.g., tetramethylammonium chloride (TMAC)), trimethybenzylammonium chloride (TMBAC), tetraethylphosphonium chloride (TEPC), triethylbenzylammonium chloride (TEBAC), tetra-n-propylammonium chloride (TPAC), tri-n-butylbenzylammonium chloride (TBBAC), tetra-n-butylphosphonium chloride (TBPC), (3-((3-cholamidopropyl) dimethylammonio)-1-propanesulfonate) (3-((3-cholamidopropyl) dimethylammonio)-1-propanesulfonate) (CHAPS detergent), and choline dihydrogen phosphate (choline DHP). The composition may include a zwitterionic catalyst and/or a zwitterionic detergent.

When the sample is over-fixed, it can be rendered less fixed by using a method of de-crosslinking disclosed herein. When the sample is over-fixed, it can be rendered more fixed by using a method of fixation disclosed herein. A particular fixed sample can be de-crosslinked, and the level of fixation of the de-crosslinked sample can be assessed using a method disclosed herein (e.g., using a nucleic acid stain and/or an actin stain), and depending on the assessment, the de-crosslinked sample can be fixed or additionally de-crosslinked. Likewise, a particular fixed sample can be additionally fixed, and the level of fixation of the additionally fixed sample can be assessed using a method disclosed herein (e.g., using a nucleic acid stain and/or an actin stain), and depending on the assessment, the additionally fixed sample can be de-crosslinked or further additionally fixed. The de-crosslinking or additional de-crosslinking and/or the additional fixation or further additional fixation can be performed one or more times in any suitable order, and the adjustment of the level of sample fixation can be informed by a method disclosed herein.

### VII. Analytes and Analyte Detection

In some aspects, the provided embodiments can be applied in an in situ method of analyzing nucleic acid sequences, such as fluorescent in situ hybridization (FISH)-based methods, in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to detect a signal associated with a detectable label of a nucleic acid probe that is hybridized to a target sequence of a target nucleic acid in a biological sample. For example, the quality of a biological sample may be assessed as described in Section V, optionally adjusted as described in Section VI, before an assay is performed to detect analytes in the sample.

In some aspects, the provided embodiments can be applied in single cell assays, for example, as described in Section VII(B)(e). In some aspects, the provided embodiments can be applied in spatial array based assays, for example, as described in Section VII(B)(f).

In some embodiments, provided herein are methods and compositions for sample analysis comprising contacting a fixed biological sample with a nucleic acid probe that binds to an analyte at a location in the fixed biological sample, and detecting an optical signal associated with the nucleic acid probe or a product thereof, thereby detecting the analyte at the location in the biological sample. A biological sample may comprise one or a plurality of analytes of interest. Methods for performing multiplexed assays to analyze two or more different analytes in a single biological sample are provided.

The methods, probes, and kits disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule, macromolecule, or chemical compound, including a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules (e.g., cellular nucleic acids), such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which comprises a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

A. Endogenous Analytes

In some embodiments, an analyte herein is endogenous to a biological sample and can include cellular nucleic acid analytes and non-nucleic acid analytes. Methods, probes, and kits disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods, probes, and kits disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte can comprise or be associated with a target sequence. In some embodiments, the target nucleic acid and the target sequence therein may be endogenous to the sample, generated in the sample, added to the sample, or associated with an analyte in the sample. In some embodiments, the target sequence is a single-stranded target sequence (e.g., a sequence in a rolling circle amplification product). In some embodiments, the target sequence is a single-stranded target sequence (e.g., in a probe bound directly or indirectly to the analyte). In some embodiments, the target sequence is a single-stranded target sequence in a primary probe that binds to an analyte of interest in the biological sample. In some embodiments, the target sequence is a single-stranded target sequence in an intermediate probe which directly or indirectly binds to a primary probe or product thereof, where the primary probe binds to an analyte of interest in the biological sample. In some embodiments, the target sequence is a single-stranded target sequence in a secondary probe that binds to the primary probe or product thereof. In some embodiments, the analytes comprises one or more single-stranded target sequences.

B. Analyte Detection

In some embodiments, provided herein are methods, probes, and kits for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

Disclosed herein in some aspects are labelling agents (e.g., nucleic acid probes and/or probe sets) that are introduced into a cell or used to otherwise contact a biological sample such as a tissue sample. The labelling agents include probes (e.g., the primary probes disclosed herein and/or any detectable probe disclosed herein) may comprise any of a variety of entities that can hybridize to a nucleic acid, typically by Watson-Crick base pairing, such as DNA, RNA, LNA, PNA, etc. The nucleic acid probe may comprise a hybridization region that is able to directly or indirectly bind to at least a portion of a target sequence in a target nucleic acid. The nucleic acid probe may be able to bind to a specific target nucleic acid (e.g., an mRNA, or other nucleic acids disclosed herein). In some embodiments, the nucleic acid probes may be detected using a detectable label, and/or by using secondary nucleic acid probes able to bind to the nucleic acid probes. In some embodiments, the nucleic acid probes (e.g., primary probes and/or secondary probes) are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease or exonuclease for cleavage or digestion).

In some embodiments, more than one type of primary nucleic acid probes may be contacted with a sample, e.g., simultaneously or sequentially in any suitable order, such as in sequential probe hybridization/unhybridization cycles. In some embodiments, more than one type of secondary nucleic acid probes may be contacted with a sample, e.g., simultaneously or sequentially in any suitable order, such as in sequential probe hybridization/unhybridization cycles. In some embodiments, the secondary probes may comprise probes that bind to a product of a primary probe targeting an analyte. In some embodiments, more than one type of higher order nucleic acid probes may be contacted with a sample, e.g., simultaneously or sequentially in any suitable order, such as in sequential probe hybridization/unhybridization cycles. In some embodiments, more than one type of detectably labeled nucleic acid probes may be contacted with a sample, e.g., simultaneously or sequentially in any suitable order, such as in sequential probe hybridization/unhybridization cycles. In some embodiments, the detectably labeled nucleic acid probes can be used to bind to one or more primary probes, one or more secondary probes, one or more higher order probes, one or more intermediate probes between a primary/secondary/higher order probes, and/or one or more detectably or non-detectably labeled probes (e.g., as in the case of a hybridization chain reaction (HCR), a branched DNA reaction (bDNA), or the like). In some embodiments, the plurality of probes or probe sets comprises at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid probes (e.g., primary, secondary, higher order probes, and/or detectably labeled probes) that can be contacted with a sample, e.g., simultaneously or sequentially in any suitable order. Between any of the probe contacting steps disclosed herein, the method may comprise one or more intervening reactions and/or processing steps, such as modifications of a target nucleic acid, modifications of a probe or product thereof (e.g., via hybridization, ligation, extension, amplification, cleavage, digestion, branch migration, primer exchange reaction, click chemistry reaction, crosslinking, attachment of a detectable label, activating photo-reactive moieties, etc.), removal of a probe or product thereof (e.g., cleaving off a portion of a probe and/or unhybridizing the entire probe), signal modifications (e.g., quenching, masking, photo-bleaching, signal enhancement (e.g., via FRET), signal amplification, etc.), signal removal (e.g., cleaving off or permanently inactivating a detectable label), crosslinking, de-crosslinking, and/or signal detection.

The hybridization region of the probe or probe set is a target-binding sequence (sometimes also referred to as the targeting region/sequence or the recognition region/sequence) that be positioned anywhere within the probe. For instance, the target-binding sequence of a primary probe that binds to a target nucleic acid can be 5′ or 3′ to any barcode sequence in the primary probe. Likewise, the target-binding sequence of a secondary probe (which binds to a primary probe or complement or product thereof) can be 5′ or 3′ to any barcode sequence in the secondary probe. In some embodiments, the target-binding sequence may comprise a sequence that is substantially complementary to a portion of a target nucleic acid. In some embodiments, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

The hybridization region of the probe or probe set can be used to identify a particular analyte comprising or associated with a target (e.g., comprising a target sequence). In some cases, multiple probes can be used, sequentially and/or simultaneously, that can bind to (e.g., hybridize to) different regions of the same target nucleic acid. In other examples, a probe may comprise target-binding sequences (e.g., hybridization regions) that can bind to different target nucleic acid sequences, e.g., various intron and/or exon sequences of the same gene (for detecting splice variants, for example), or sequences of different genes, e.g., for detecting a product that comprises the different target nucleic acid sequences, such as a genome rearrangement (e.g., inversion, transposition, translocation, insertion, deletion, duplication, and/or amplification).

In some embodiments, provided herein are methods, probes, and kits for analyzing endogenous analytes (e.g., RNA, ssDNA, and cell surface or intracellular proteins and/or metabolites) in a sample using one or more labelling agents.

In some embodiments, the labelling agent is an immunohistochemistry (IHC) probe that is excited at various different wavelengths. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode comprises to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety comprises one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes comprises a single species of analyte (e.g., a single species of polypeptide). In some embodiments in which the plurality of analytes comprises a single species of analyte, the analyte binding moieties of the plurality of analyte labelling agents are the same. In some embodiments in which the plurality of analytes comprises a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes comprises multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labelling agent through a labile bond (e.g., chemically labile, photolabile, thermally labile, etc.) as generally described for releasing molecules from supports elsewhere herein.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

a. Hybridization

In some embodiments, the labelling agents (e.g., probes or probes sets) described herein can be used to detect an endogenous analyte, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or an exogenous molecule such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. In some instances, various probes and probe sets can be used to generate a product comprising a target sequence that can be hybridized by one or more detectable probes. In some instances, a probe or probe set disclosed herein is a circularizable probe or probe set comprising a barcode region comprising one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

b. Ligation

In some embodiments, a product of an endogenous analyte and/or a labelling agent is a ligation product that may comprise a target sequence that can be hybridized by one or more probes described herein for detection of analytes. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent or probe, for example, the circularization of a circularizable probe or probe set upon hybridization to a target sequence. The target sequence can be comprised in an endogenous analyte (e.g., nucleic acid such as genomic DNA or mRNA) or a product thereof (e.g., cDNA from a cellular mRNA transcript), or in a labelling agent (e.g., the reporter oligonucleotide) or a product thereof.

In some embodiments, provided herein is a labelling agent comprising a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety.

In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, a circular or circularizable probe or probe set may be used to analyze a reporter oligonucleotide, which may generated using proximity ligation or be subjected to proximity ligation. In some examples, the reporter oligonucleotide of a labelling agent that specifically recognizes a protein can be analyzed using in situ hybridization (e.g., sequential hybridization) and/or in situ sequencing (e.g., using circular or circularizable probes and rolling circle amplification of circular or circularized probes). Further, the reporter oligonucleotide of the labelling agent and/or a complement thereof and/or a product (e.g., a hybridization product, a ligation product, an extension product (e.g., by a DNA or RNA polymerase), a replication product, a transcription/reverse transcription product, and/or an amplification product) thereof can be recognized by another labelling agent and analyzed.

In some embodiments, an analyte (a nucleic acid analyte or non-nucleic acid analyte) can be specifically bound by two labelling agents (e.g., antibodies) each of which is attached to a reporter oligonucleotide (e.g., DNA) that can participate in ligation, replication, and sequence decoding reactions, e.g., using a probe or probe set (e.g., a padlock probe, a SNAIL probe set, a circular probe, a gapped padlock probe, or a gapped padlock probe and a connector). In some embodiments, the probe set may comprise two or more probe oligonucleotides, each comprising a region that is complementary to each other. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template PCR amplification, as described for example in Soderberg et al., Methods. (2008), 45(3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Application Publication No. 2002/0051986, the entire contents of which are incorporated herein by reference. In some embodiments, two analytes in proximity can be specifically bound by two labelling agents (e.g., antibodies) each of which is attached to a reporter oligonucleotide (e.g., DNA) that can participate, when in proximity when bound to their respective targets, in ligation, replication, and/or sequence decoding reactions.

In some embodiments, one or more reporter oligonucleotides (and optionally one or more other nucleic acid molecules such as a connector) aid in the ligation of the probe. Upon ligation, the probe may form a circularized probe. In some embodiments, one or more suitable probes can be used and ligated, wherein the one or more probes comprise a sequence that is complementary to the one or more reporter oligonucleotides (or portion thereof). The probe may comprise one or more barcode sequences. In some embodiments, the one or more reporter oligonucleotide may serve as a primer for rolling circle amplification (RCA) of the circularized probe. In some embodiments, a nucleic acid other than the one or more reporter oligonucleotide is used as a primer for rolling circle amplification (RCA) of the circularized probe. For example, a nucleic acid capable of hybridizing to the circularized probe at a sequence other than sequence(s) hybridizing to the one or more reporter oligonucleotide can be used as the primer for RCA. In other examples, the primer in a SNAIL probe set is used as the primer for RCA.

In some embodiments, one or more analytes can be specifically bound by two primary antibodies, each of which is in turn recognized by a secondary antibody each attached to a reporter oligonucleotide (e.g., DNA). Each nucleic acid molecule can aid in the ligation of the probe to form a circularized probe. In some instances, the probe can comprise one or more barcode sequences. Further, the reporter oligonucleotide may serve as a primer for rolling circle amplification of the circularized probe. The nucleic acid molecules, circularized probes, and RCA products can be analyzed using any suitable method disclosed herein for in situ analysis.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

c. Primer Extension

In some embodiments, a primer extension product of an analyte, a labelling agent, a probe or probe set bound to the analyte (e.g., bound to genomic DNA, mRNA, or cDNA), or a probe or probe set bound to the labelling agent (e.g., bound to one or more reporter oligonucleotides from the same or different labelling agents). Any of such products of extension may comprise a target sequence that can be hybridized by the plurality of probes or probe sets described herein.

In some embodiments, the plurality of probes or probe sets comprises a primer. A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) include linear RCA, a branched RCA, a dendritic RCA, or any combination thereof. See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97(18):10113-9, 2000; Faruqi et al, BMC Genomics 2:4, 2000; Nallur et al, Nucl. Acids Res. 29:e118, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801, all of which are incorporated by reference. Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N⁶-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, US 2016/0024555, US 2018/0251833, US 2017/0219465, U.S. Pat. Nos. 10,138,509, 10,494,662, 11,078,520, 11,299,767, 10,266,888, 11,118,220, US 2021/0363579, US 2021/0324450, and US 2021/0215581, all of which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, the detection of numerous different analytes may use a RCA-based detection system, e.g., where the signal is provided by generating a target sequence from a circular RCA template which is provided or generated in the assay, and the target sequence is detected to detect the corresponding analyte. The target sequence may thus be regarded as a reporter which is detected to detect the target analyte. However, the RCA template may also be regarded as a reporter for the target analyte; the target sequence is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g. a reagent in a detection assay), which is used as a reporter for the assay, or a part of a reporter, or signal-generation system. The RCA template used to generate the target sequence may thus be a circular (e.g. circularized) reporter nucleic acid molecule, namely from any RCA-based detection assay which uses or generates a circular nucleic acid molecule as a reporter for the assay. Since the RCA template generates the target sequence reporter, it may be viewed as part of the reporter system for the assay.

In some embodiments, a product herein comprises a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a target-binding region in a probe may be a hybridization complex formed of a cellular nucleic acid in a sample and an exogenously added nucleic acid probe or a product generated therefrom. The exogenously added nucleic acid probe (e.g., plurality of probes or probe sets) may comprise an overhang that does not hybridize to the cellular nucleic acid but hybridizes to another probe (e.g., an intermediate probe).

In some embodiments, the labelling agents may bind or hybridize to a target. In some instances, a target comprises a target sequence for a probe or probe set. In some instances, the plurality of probes or probe sets may be used to generate a product comprising signal amplification components. In some instances, the amplification comprises one or more probe hybridizations and generation of amplified signals associated with the labelling agents (e.g., probes). Exemplary signal amplification methods include targeted assembly of branched structures (e.g., bDNA). In some instances, detection of nucleic acids sequences in situ includes combination of the sequential decoding methods described herein with an assembly for branched signal amplification using the nucleic acid probes provided herein. In some instances, the assembly complex comprises an amplifier hybridized directly or indirectly (via one or more oligonucleotides) to a sequence of a cellular nucleic acid.

After contacting the biological sample with a plurality of labelling agents (e.g., probes or probe sets), the probes may be directly detected by determining detectable labels (if present), and/or detected by using one or more other probes that bind directly or indirectly to the plurality of probes or probe sets or products thereof. The one or more other probes may comprise a detectable label. For instance, a primary nucleic acid probe can bind to a target nucleic acid in the sample, and a secondary nucleic acid probe can be introduced to bind to the primary nucleic acid probe, where the secondary nucleic acid probe or a product thereof can then be detected using detectable probes (e.g., detectably labeled probes). Higher order probes that directly or indirectly bind to the secondary nucleic acid probe or product thereof may also be used, and the higher order probes or products thereof can then be detected using detectably labeled probes.

In some instances, a secondary nucleic acid probe binds to a primary nucleic acid probe directly hybridized to the target nucleic acid. A secondary nucleic acid probe (e.g., a first detectable probe or a second detectable probe disclosed herein) may contain a recognition sequence able to bind to or hybridize with a primary nucleic acid probe (e.g., probes or probe sets disclosed herein) or a product thereof (e.g., an RCA product), e.g., at a barcode sequence or portion(s) thereof of the probes or probe sets or products thereof. In some embodiments, a secondary nucleic acid probe may bind to a combination of barcode sequences (which may be continuous or spaced from one another) in the probes or probe sets, a product thereof. In some embodiments, the binding is specific, or the binding may be such that a recognition sequence preferentially binds to or hybridizes with only one of the barcode sequences or complements thereof that are present. The secondary nucleic acid probe may also contain one or more detectable labels. If more than one secondary nucleic acid probe is used, the detectable labels may be the same or different.

The recognition sequences may be of any length, and multiple recognition sequences in the same or different secondary nucleic acid probes may be of the same or different lengths. If more than one recognition sequence is used, the recognition sequences may independently have the same or different lengths. For instance, the recognition sequence may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence may be no more than 48, no more than 40, no more than 32, no more than 24, no more than 16, no more than 12, no more than 10, no more than 8, or no more than 6 nucleotides in length. Combinations of any of these are also possible, e.g., the recognition sequence may have a length of between 5 and 8, between 6 and 12, or between 7 and 15 nucleotides, etc. In some embodiments, the recognition sequence is of the same length as a barcode sequence or complement thereof of a primary nucleic acid probe or a product thereof. In some embodiments, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to the barcode sequence or complement thereof.

In some embodiments, the probes or probe sets, or an intermediate probe, may also comprise 1, 2, 3, 4, 5, 6, 7, 8, 9, 10, 11, 12, 13, 14, 15 or more, 20 or more, 32 or more, 40 or more, or 50 or more barcode sequences. As an illustrative example, a first probe may contain a first target-binding sequence, a first barcode sequence, and a second barcode sequence, while a second, different probe may contain a second target-binding sequence (that is different from the first target-binding sequence in the first probe), the same first barcode sequence as in the first probe, but a third barcode sequence instead of the second barcode sequence. Such probes may thereby be distinguished by determining the various barcode sequence combinations present or associated with a given probe at a given location in a sample.

In some embodiments, the nucleic acid probes disclosed herein may be made using only 2 or only 3 of the 4 bases, such as leaving out all the “G”s and/or leaving out all of the “C”s within the probe. Sequences lacking either “G”s or “C”s may form very little secondary structure, and can contribute to more uniform, faster hybridization in certain embodiments.

In some embodiments, a nucleic acid probe disclosed herein may contain a detectable label such as a fluorophore. In some embodiments, one or more probes of a plurality of nucleic acid probes used in an assay may lack a detectable label, while one or more other probes in the plurality each comprises a detectable label selected from a limited pool of distinct detectable labels (e.g., red, green, yellow, and blue fluorophores), and the absence of detectable label may be used as a separate “color.” As such, detectable labels are not required in all cases. In some embodiments, a primary nucleic acid probe disclosed herein lacks a detectable label. While a detectable label may be incorporated into an amplification product of a probe, such as via incorporation of a modified nucleotide into an RCA product of a circularized probe, the amplification product itself in some embodiments is not detectably labeled. In some embodiments, a probe that binds to the primary nucleic acid probe or a product thereof (e.g., a secondary nucleic acid probe that binds to a barcode sequence or complement thereof in the primary nucleic acid probe or product thereof) comprises a detectable label and may be used to detect the primary nucleic acid probe or product thereof. In some embodiments, a secondary nucleic acid probe disclosed herein lacks a detectable label, and a detectably labeled probe that binds to the secondary nucleic acid probe or a product thereof (e.g., at a barcode sequence or complement thereof in the secondary nucleic acid probe or product thereof) can be used to detect the second nucleic acid probe or product thereof. In some embodiments, signals associated with the detectably labeled probes (e.g., the first detectable probe which is detectably labelled, the second detectable probe which is detectably labelled, a detectably labeled probe that binds to the first detectable probe which itself is not detectably labelled, or a detectably labeled probe that binds to the second detectable probe which itself is not detectably labelled) can be used to detect one or more barcode sequences in the secondary probe and/or one or more barcode sequences in the primary probe, e.g., by using sequential hybridization of detectably labeled probes, sequencing-by-ligation, and/or sequencing-by-hybridization. In some embodiments, the barcode sequences (e.g., in the secondary probe and/or in the primary probe) are used to combinatorially encode a plurality of analytes of interest. As such, signals associated with the detectably labeled probes at particular locations in a biological sample can be used to generate distinct signal signatures that each corresponds to an analyte in the sample, thereby identifying the analytes at the particular locations, e.g., for in situ spatial analysis of the sample.

In some embodiments, probes or probe sets described herein comprises one or more other components, such as one or more primer binding sequences (e.g., to allow for enzymatic amplification of probes), enzyme recognition sequences (e.g., for endonuclease cleavage), or the like. The components of the nucleic acid probe may be arranged in any suitable order3%%

In some aspects, targets (e.g., analytes) are targeted by labelling agents (e.g., probes or probe sets) described herein, which are barcoded through the incorporation of one or more barcode sequences (e.g., sequences that can be detected or otherwise “read”) and binds the targeted analyte. In some aspects, the probes or probe sets described herein are in turn targeted by secondary probes e.g., intermediate probes, which are also barcoded through the incorporation of one or more barcode sequences that are separate from a recognition sequence in a secondary probe that directly or indirectly binds the probes or probe sets described herein or a product thereof. In some embodiments, a secondary probe may bind to a barcode sequence in the primary probe. In some aspects, tertiary probes and optionally even higher order probes may be used to target the secondary probes, e.g., at a barcode sequence or complement thereof in a secondary probe or product thereof. In some embodiments, the tertiary probes and/or even higher order probes may comprise one or more barcode sequences and/or one or more detectable labels. In some embodiments, a tertiary probe is a detectably labeled probe that hybridizes to a barcode sequence (or complement thereof) of a secondary probe (or product thereof). In some embodiments, through the detection of signals associated with detectably labeled probes in a sample, the location of one or more analytes in the sample and the identity of the analyte(s) can be determined. In some embodiments, the presence/absence, absolute or relative abundance, an amount, a level, a concentration, an activity, and/or a relation with another analyte of a particular analyte can be analyzed in situ in the sample.

In some embodiments, provided herein are labelling agents (e.g., probes or probe sets), and assay methods to couple target nucleic acid detection, signal amplification (e.g., through nucleic acid amplification such as RCA, and/or hybridization of a plurality of detectably labeled probes, such as in hybridization chain reactions and the like, e.g., described in Section III-C), and decoding of the barcodes.

In some aspects, probes or probe sets described herein, or intermediate probes (e.g., a secondary probe, and/or a higher order probe) can be selected from the group consisting of a circular probe, a circularizable probe, and a linear probe. In some embodiments, a circular probe can be one that is pre-circularized prior to hybridization to a target nucleic acid and/or one or more other probes. In some embodiments, a circularizable probe can be one that can be circularized upon hybridization to a target nucleic acid and/or one or more other probes such as a splint. In some embodiments, a linear probe can be one that comprises a target recognition sequence and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) is non-hybridizing to the target nucleic acid but may hybridize to one another and/or one or more other probes, such as detectably labeled probes.

Specific probe designs can vary depending on the application. For instance, probes or probe sets described herein (e.g., a primary probe,) or a secondary probe, and/or a higher order probe disclosed herein can comprise a circularizable probe that does not require gap filling to circularize upon hybridization to a template (e.g., a target nucleic acid and/or a probe such as a splint), a gapped circularizable probe (e.g., one that requires gap filling to circularize upon hybridization to a template), an L-shaped probe (e.g., one that comprises a target recognition sequence and a 5′ or 3′ overhang upon hybridization to a target nucleic acid or a probe), a U-shaped probe (e.g., one that comprises a target recognition sequence, a 5′ overhang, and a 3′ overhang upon hybridization to a target nucleic acid or a probe), a V-shaped probe (e.g., one that comprises at least two target recognition sequences and a linker or spacer between the target recognition sequences upon hybridization to a target nucleic acid or a probe), a probe or probe set for proximity ligation (such as those described in U.S. Pat. Nos. 7,914,987 and 8,580,504 incorporated herein by reference in their entireties, and probes for Proximity Ligation Assay (PLA) for the simultaneous detection and quantification of nucleic acid molecules and protein-protein interactions), or any suitable combination thereof. In some embodiments, a primary probe, a secondary probe, and/or a higher order probe disclosed herein can comprise a probe that is ligated to itself or another probe using DNA-templated and/or RNA-templated ligation. In some embodiments, a primary probe, a secondary probe, and/or a higher order probe disclosed herein can be a DNA molecule and can comprise one or more other types of nucleotides, modified nucleotides, and/or nucleotide analogues, such as one or more ribonucleotides. In some embodiments, the ligation can be a DNA ligation on a DNA template. In some embodiments, the ligation can be a DNA ligation on an RNA template, and the probes can comprise RNA-templated ligation probes. In some embodiments, a primary probe, a secondary probe, and/or a higher order probe disclosed herein can comprise a padlock-like probe or probe set, such as one described in US 2019/0055594, US 2021/0164039, US 2016/0108458, or US 2020/0224243, each of which is incorporated herein by reference in its entirety. Any suitable combination of the probe designs described herein can be used.

In some embodiments, probes or probe sets described herein (e.g., a primary probe,) or a secondary probe, and/or a higher order probe disclosed herein can comprise two or more parts. In some cases, a probe can comprise one or more features of and/or be modified based on: a split FISH probe or probe set described in WO 2021/167526A1 or Goh et al., “Highly specific multiplexed RNA imaging in tissues with split-FISH,” Nat Methods 17(7):689-693 (2020), which are incorporated herein by reference in their entireties; a Z-probe or probe set, such as one described in U.S. Pat. No. 7,709,198 B2, U.S. Pat. No. 8,604,182 B2, U.S. Pat. No. 8,951,726 B2, U.S. Pat. No. 8,658,361 B2, or Tripathi et al., “Z Probe, An Efficient Tool for Characterizing Long Non-Coding RNA in FFPE Tissues,” Noncoding RNA 4(3):20 (2018), which are incorporated herein by reference in their entireties; an HCR initiator or amplifier, such as one described in U.S. Pat. No. 7,632,641 B2, US 2017/0009278 A1, U.S. Pat. No. 10,450,599 B2, Dirks and Pierce, “Triggered amplification by hybridization chain reaction,” PNAS 101(43):15275-15278 (2004), Chemeris et al., “Real-time hybridization chain reaction,” Dokl. Biochem 419:53-55 (2008), Niu et al., “Fluorescence detection for DNA using hybridization chain reaction with enzyme-amplification,” Chem Commun (Camb) 46(18):3089-91 (2010), Choi et al., “Programmable in situ amplification for multiplexed imaging of mRNA expression,” Nat Biotechnol 28(11):1208-12 (2010), Song et al., “Hybridization chain reaction-based aptameric system for the highly selective and sensitive detection of protein,” Analyst 137(6):1396-401 (2012), Choi et al., “Third-generation in situ hybridization chain reaction: multiplexed, quantitative, sensitive, versatile, robust,” Development 145(12): dev165753 (2018), or Tsuneoka and Funato, “Modified in situ Hybridization Chain Reaction Using Short Hairpin DNAs,” Front Mol Neurosci 13:75 (2020), which are incorporated herein by reference in their entireties; a PLAYR probe or probe set, such as one described in US 2016/0108458 A1 or Frei et al., “Highly multiplexed simultaneous detection of RNAs and proteins in single cells,” Nat Methods 13(3):269-75 (2016), which are incorporated herein by reference in their entireties; a PLISH probe or probe set, such as one described in US 2020/0224243 A1 or Nagendran et al., “Automated cell-type classification in intact tissues by single-cell molecular profiling,” eLife 7:e30510 (2018), which are incorporated herein by reference in their entireties; a RollFISH probe or probe set such as one described in Wu et al., “RollFISH achieves robust quantification of single-molecule RNA biomarkers in paraffin-embedded tumor tissue samples,” Commun Biol 1, 209 (2018), which is hereby incorporated by reference in its entirety; a MERFISH probe or probe set, such as one described in US 2022/0064697 A1 or Chen et al., “Spatially resolved, highly multiplexed RNA profiling in single cells,” Science 348(6233):aaa6090 (2015), which are incorporated herein by reference in their entireties; or a primer exchange reaction (PER) probe or probe set, such as one described in US 2019/0106733 A1, which is hereby incorporated by reference in its entirety.

In some embodiments, probes or probe sets described herein comprise one or more features and/or is modified to allow for generation and detection of a first signal that does not comprise a nucleic acid amplification step (e.g., the first signal can be an smFISH signal). In some instances, the probes or probe sets described herein for each target comprises probes directly hybridize to multiple regions (e.g., sequences) of the same transcript. In some embodiments, the probes or probe sets described herein comprise a circular probe or circularizable probe or probe set comprises one or more features and/or is modified to allow for generation and detection of a second signal that comprises an amplification step (e.g., extension and/or amplification catalyzed by a polymerase).

Any suitable circularizable probe or probe set may be used to generate the RCA template which is used to generate the RCA product. By “circularizable” is meant that the probe or reporter (the RCA template) is in the form of a linear molecule having ligatable ends which may circularized by ligating the ends together directly or indirectly, e.g. to each other, or to the respective ends of an intervening (“gap”) oligonucleotide or to an extended 3′ end of the circularizable RCA template. A circularizable template may also be provided in two or more parts, namely two or more molecules (e.g. oligonucleotides) which may be ligated together to form a circle. When said RCA template is circularizable it is circularized by ligation prior to RCA. Ligation may be templated using a ligation template. The circularizable RCA template (or template part or portion) may comprise at its respective 3′ and 5′ ends regions of complementarity to corresponding cognate complementary regions (or binding sites) in the ligation template, which may be adjacent where the ends are directly ligated to each other, or non-adjacent, with an intervening “gap” sequence, where indirect ligation is to take place.

In some embodiments, probes or probe sets disclosed herein can be pre-assembled from multiple components, e.g., prior to contacting the probe with a target nucleic acid or a sample. In some embodiments, a nucleic acid probe disclosed herein can be assembled during and/or after contacting a target nucleic acid or a sample with multiple components. In some embodiments, a nucleic acid probe disclosed herein is assembled in situ in a sample. In some embodiments, the multiple components can be contacted with a target nucleic acid or a sample in any suitable order and any suitable combination. For instance, a first component and a second component can be contacted with a target nucleic acid, to allow binding between the components and/or binding between the first and/or second components with the target nucleic acid. Optionally a reaction involving either or both components and/or the target nucleic acid, between the components, and/or between either one or both components and the target nucleic acid can be performed, such as hybridization, ligation, primer extension and/or amplification, chemical or enzymatic cleavage, click chemistry, or any combination thereof. In some embodiments, a third component can be added prior to, during, or after the reaction. In some embodiments, a third component can be added prior to, during, or after contacting the sample with the first and/or second components. In some embodiments, the first, second, and third components can be contacted with the sample in any suitable combination, sequentially or simultaneously. In some embodiments, the nucleic acid probe can be assembled in situ in a stepwise manner, each step with the addition of one or more components, or in a dynamic process where all components are assembled together. One or more removing steps, e.g., by washing the sample such as under stringent conditions, may be performed at any point during the assembling process to remove or destabilize undesired intermediates and/or components at that point and increase the chance of accurate probe assembly and specific target binding of the assembled probe.

In some aspects, the methods provided herein comprise performing rolling circle amplification of a circular probe or a circularized probe generated from a circularizable probe or probe set.

In some embodiments, a probe disclosed herein can comprise a 5′ flap which may be recognized by a structure-specific cleavage enzyme, e.g. an enzyme capable of recognizing the junction between a single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to a target, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiments, cleavage of the additional sequence 5′ to the first target-specific binding site is performed by a structure-specific cleavage enzyme, e.g. a Flap endonuclease. Suitable Flap endonucleases are described in Ma et al. 2000. JBC 275, 24693-24700 and in US 2020/0224244 and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g. dinucleotides or trinucleotides), or cleaving the entire 5′ single-stranded region, e.g. as described in Lyamichev et al. 1999. PNAS 96, 6143-6148 for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

A target sequence for a probe disclosed herein may be comprised in any analyte (e.g., target) disclose herein, including an endogenous analyte (e.g., a viral or cellular nucleic acid), a labelling agent, or a product of an endogenous analyte and/or a labelling agent. In some embodiments, a target sequence for a probe disclosed herein comprises one or more ribonucleotides.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, a barcode may be a barcode region. In some embodiments, a barcode comprises two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, barcodes or complements thereof (e.g., barcode sequences or complements thereof comprised by the labelling agents (e.g., probes) disclosed herein or products thereof) can be analyzed (e.g., detected or sequenced) using any suitable method or technique, including those described herein, such as sequencing by synthesis (SBS), sequencing by ligation (SBL), or sequencing by hybridization (SBH). In some instances, barcoding schemes and/or barcode detection schemes as described in RNA sequential probing of targets (RNA SPOTs), single-molecule fluorescent in situ hybridization (smFISH), multiplexed error-robust fluorescence in situ hybridization (MERFISH) or sequential fluorescence in situ hybridization (seqFISH+) can be used. In any of the preceding implementations, the methods provided herein can include analyzing the barcodes by sequential hybridization and detection with a plurality of labelled probes (e.g., detection probes (e.g., detection oligos) or barcode probes). In some instances, the barcode detection steps can be performed as described in hybridization-based in situ sequencing (HybISS). In some instances, probes can be detected and analyzed (e.g., detected or sequenced) as performed in fluorescent in situ sequencing (FISSEQ), or as performed in the detection steps of the spatially-resolved transcript amplicon readout mapping (STARmap) method. In some instances, signals associated with an analyte can be detected as performed in sequential fluorescent in situ hybridization (seqFISH).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCA products are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub. 20210164039, which are hereby incorporated by reference in their entirety.

d. Analyte Detection Using Optical Signals

Provided herein are a plurality of detectable probes contacted with the biological sample for detecting a plurality of signals associated with a plurality of targets in the biological sample. In some embodiments, the detectable probes are detectably labeled or comprise a detectable label. The terms “label” and “detectable label” comprise a directly or indirectly detectable moiety that is associated with (e.g., conjugated to) a molecule to be detected, e.g., a nucleic acid molecule that comprises a detectable label. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes (radioactive isotopes), fluorophores, fluorescers, chemiluminescent compounds, bioluminescent compounds, dyes, enzymes, enzyme substrates, enzyme cofactors, enzyme inhibitors, chromophores, dyes, metal ions, metal sols, ligands (e.g., biotin or haptens) and the like.

In some aspects, the detectable label comprises a luminophore. In some embodiments, the luminophore is a fluorophore. The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes (AlexaFluors), fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas RedY-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

In some embodiments, the detectable label comprises an infrared fluorophore. An “infrared fluorophore” emits infrared light. In some embodiments, the infrared fluorophore has a longer excitation wavelength than a traditional fluorophore.

Examples of detectable labels comprise, but are not limited to, various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

In some embodiments, one or more detectable labels can be attached to a labelling agent or nucleic acid probe disclosed herein. The one or more detectable labels can be incorporated during nucleic acid polymerization or amplification (e.g., Cy5®-labeled nucleotides, such as Cy5®-dCTP). Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores can include those described in Henegariu et al. (2000) Nature Biotechnol. 18:345, incorporated herein by reference.

In some embodiments, one or more detectable labels can be attached via post-synthetic attachment. Fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62)

Biotin, or a derivative thereof, may also be used as a label on a nucleic acid molecule, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as an Fab.

Other suitable labels for use in the methods provided herein may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleic acid molecule (e.g., detectable probe) can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, each of which is herein incorporated by reference in its entirety. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

In some embodiments, the detectable label is detected in situ. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some aspects, the method includes detection of the probe or probe set hybridized to the target (e.g., target sequence) or any products generated therefrom or a derivative thereof. In any of the embodiments herein, the method can further comprise imaging the biological sample to detect a ligation product or a circularized probe or product thereof. In any of the embodiments herein, a sequence of the ligation product, rolling circle amplification product, or other generated product can be analyzed in situ in the biological sample. In any of the embodiments herein, the imaging can comprise detecting a signal associated with a fluorescently labeled probe that directly or indirectly binds to a rolling circle amplification product of the circularized probe. In any of the embodiments herein, the sequence of the sequence of the ligation product, rolling circle amplification product, or other generated product can be analyzed by sequential hybridization, sequencing by hybridization, sequencing by ligation, sequencing by synthesis, sequencing by binding, or a combination thereof.

In any of the embodiments herein, a sequence associated with the target nucleic acid or the probes or probe sets described herein can comprise one or more barcode sequences or complements thereof. In any of the embodiments herein, the sequence of the rolling circle amplification product can comprise one or more barcode sequences or complements thereof. In any of the embodiments herein, a probe can comprise one or more barcode sequences or complements thereof. In any of the embodiments herein, the one or more barcode sequences can comprise a barcode sequence corresponding to the target nucleic acid. In any of the embodiments herein, the one or more barcode sequences can comprise a barcode sequence corresponding to the sequence of interest, such as variant(s) of a single nucleotide of interest.

In any of the embodiments herein, the detecting step can comprise contacting the biological sample with one or more detectable probes that directly or indirectly hybridize to the rolling circle amplification product, and dehybridizing the one or more detectable probes from the rolling circle amplification product. In any of the embodiments herein, the contacting and dehybridizing steps can be repeated with the one or more detectable probes and/or one or more other detectable probes that directly or indirectly hybridize to the rolling circle amplification product.

In any of the embodiments herein, the detecting step can comprise contacting the biological sample with one or more first detectable probes that directly hybridize to the plurality of probes or probe sets. In some instances, the detecting step can comprise contacting the biological sample with one or more first detectable probes that indirectly hybridize to the plurality of probes or probe sets. In any of the embodiments herein, the detecting step can comprise contacting the biological sample with one or more first detectable probes that directly or indirectly hybridize to the plurality of probes or probe sets.

In any of the embodiments herein, the detecting step can comprise contacting the biological sample with one or more intermediate probes that directly or indirectly hybridize to the plurality of probes or probe sets, rolling circle amplification product generated using the plurality of probes or probe sets, wherein the one or more intermediate probes are detectable using one or more detectable probes. In any of the embodiments herein, the detecting step can further comprise dehybridizing the one or more intermediate probes and/or the one or more detectable probes from the rolling circle amplification product or the plurality of probes or probe sets. In any of the embodiments herein, the contacting and dehybridizing steps can be repeated with the one or more intermediate probes, the one or more detectable probes, one or more other intermediate probes, and/or one or more other detectable probes.

In some embodiments, the detection may be spatial, e.g., in two or three dimensions. In some embodiments, the detection may be quantitative, e.g., the amount or concentration of a primary nucleic acid probe (and of a target nucleic acid) may be determined. In some embodiments, the plurality of probes or probe sets (e.g., primary probes), secondary probes, higher order probes, and/or detectable probes may comprise any of a variety of entities able to hybridize a nucleic acid, e.g., DNA, RNA, LNA, and/or PNA, etc., depending on the application.

In some embodiments, a method disclosed herein may also comprise one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, the RCA product generated using a method disclosed herein can be detected in with a method that comprises signal amplification. In some embodiments, signal amplification may comprise use of the plurality of probes or probe sets.

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, US 2022/0026433, US 2022/0128565, and US 2021/0222234, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product. Exemplary methods and compositions for LO-HCR are described in US 2021/0198723, incorporated herein by reference in its entirety.

In some embodiments, detection of nucleic acids sequences in situ may comprise an assembly for branched signal amplification. In some embodiments, the assembly complex comprises an amplifier hybridized directly or indirectly (via one or more oligonucleotides) to a probe or probe set. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, the plurality of probes or probe sets can be detected in with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising the plurality of probes or probe sets described herein. In various embodiments, the plurality of probes or probe sets may be contacted with a plurality of concatemer primers and a plurality of labeled probes. see e.g., U.S. Pat. Pub. No. US20190106733, which is incorporated herein by reference, for exemplary molecules and PER reaction components.

In some embodiments, the methods comprise sequencing all or a portion of the amplification product, such as one or more barcode sequences present in the amplification product, e.g., via DNA sequencing.

In some embodiments, the analysis and/or sequence determination comprises sequencing all or a portion of the amplification product or the probe(s) and/or in situ hybridization to the amplification product or the probe(s). In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is incorporated herein by reference, for exemplary probes and HCR reaction components. In some embodiments, the detection or determination comprises hybridizing to the amplification product a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some aspects, the provided methods comprise imaging the amplification product (e.g., amplicon) and/or one or more portions of the plurality of probes or probe sets, for example, via binding of the detection probe and detecting the detectable label. In some embodiments, the detection probe comprises a detectable label that can be measured and quantitated. The terms “label” and “detectable label” comprise a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect the plurality of probes or probe sets and/or amplification products (e.g., amplicon) described herein. In some embodiments, the methods involve incubating the detectable probe containing the detectable label with the sample, washing unbound detectable probe, and detecting the label, e.g., by imaging.

In some aspects, the detecting comprises performing microscopy, scanning mass spectrometry or other imaging techniques described herein. In such aspects, the detecting comprises determining a signal, e.g., a fluorescent signal. In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932. Exemplary techniques for in situ sequencing comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121). In some cases, sequencing can be performed after the analytes are released from the biological sample.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232.

In some embodiments, sequence analysis of nucleic acids (e.g., nucleic acids such as RCA products comprising barcode sequences) can be performed by sequential hybridization (e.g., sequencing by hybridization and/or sequential in situ fluorescence hybridization). Sequential fluorescence hybridization can involve sequential hybridization of detection probes comprising an oligonucleotide and a detectable label. In some embodiments, a method disclosed herein comprises sequential hybridization of the detectable probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectable probes are described in US 2019/0161796, US 2020/0224244, US 2022/0010358, US 2021/0340618, and WO 2021/138676, all of which are incorporated herein by reference.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597.

In some embodiments, the barcodes of the probes (e.g., plurality of probes or probe sets) or complements or products thereof are targeted by detectably labeled detection oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides or detectable probes). Exemplary decoding schemes are described in Eng et al., “Transcriptome-scale Super-Resolved Imaging in Tissues by RNA SeqFISH+,” Nature 568(7751):235-239 (2019); Chen et al., Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

In some aspects, the provided embodiments can be applied to an in situ method of analyzing target nucleic acid sequences (e.g., RNAs) and/or other targets (e.g., proteins) in intact tissues or samples in which the spatial information has been preserved. In some aspects, the embodiments can be applied to a biological sample assessed for quality as described in Section V and assayed using an imaging or detection method for multiplexed analysis of nucleic acids and/or other targets (e.g., proteins) in the assessed biological sample. In some aspects, the provided embodiments can be applied to a biological sample assessed for quality as described in Section V and used to identify or detect regions and/or sequences of interest in target nucleic acids in the assessed biological sample.

In some cases, analysis is performed on one or more images captured, and may comprise processing the image(s) and/or quantifying signals observed. In some embodiments, images of signals from different fluorescent and/or non-fluorescent channels and/or detectable probe hybridization cycles can be compared and analyzed. In some embodiments, images of signals (or absence thereof) at a particular location in a sample from different fluorescent channels and/or sequential detectable probe hybridization cycles can be aligned to analyze an analyte at the location. For instance, a particular location in a sample can be tracked and signal spots from sequential hybridization cycles can be analyzed to detect a target polynucleotide sequence (e.g., an associated barcode sequence or subsequence thereof) at the location. The analysis may comprise processing information of one or more cell types, one or more types of analytes, a number or level of analyte, and/or a number or level of cells detected in a particular region of the sample. In some embodiments, the analysis comprises detecting a sequence e.g., a barcode sequence present in an amplification product at a location in the sample. In some embodiments, the analysis includes quantification of puncta (e.g., if amplification products are detected). In some cases, the analysis includes determining whether particular cells and/or signals are present that correlate with one or more analytes from a particular panel. In some instances, the analysis includes using single cell segmentation and resolution to determine cell type frequencies in a region of interest of a sample. In some embodiments, the obtained information may be compared to a positive and negative control, to another selected region of interest, or to a threshold of a feature to determine if the region of interest exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

e. Single Cell Analysis

A fixed biological sample disclosed herein can be used for single cell analysis after assessing the quality (e.g., level of fixation) of the sample. The fixed biological sample can be de-crosslinked or additionally fixed before contacting with nucleic acid probes for single cell analysis.

In some embodiments, an assay can be performed for single cell whole transcriptome gene expression and multiplexing capabilities to profile hundreds to a million cells. Fixation during sample collection preserves fragile biology and greatly streamlines downstream workflows for processing, allows for large longitudinal studies, removes constraints from transport and storage of samples, and minimizes variability by matching samples. In some embodiments, sample fixation locks in cells states and preserve fragile samples and further allows for detection of transitionary cell types that may be missed without fixation. In some embodiments, chromium fixed RNA profiling allows comprehensive scalable solutions to measure gene expression in single cell and nuclei suspensions that are fixed with formaldehyde. Intracellular protein staining involves cell fixation by using paraformaldehyde (PFA) due to the improved signal-to-background ratio in intracellular staining and preservation of intracellular structure integrity.

In some aspects, methods of the present disclosure include a method of analyzing a sample comprising a nucleic acid molecule, the method comprising (a) providing: (i) the sample comprising the nucleic acid molecule(s), wherein the nucleic acid molecule(s) comprises a first target region and a second target region, and wherein the first target region and the second target region are both disposed on a strand of the nucleic acid molecule(s); (ii) a first probe comprising a first probe sequence and a first barcode sequence, wherein the first probe sequence of the first probe is complementary to the first target region of the nucleic acid molecule(s); and (iii) a second probe comprising a second probe sequence, wherein the second probe sequence of the second probe is complementary to the second target region of the nucleic acid molecule(s); (b) subjecting the sample to conditions sufficient to (i) hybridize the first probe sequence of the first probe to the first target region of the nucleic acid molecule(s), and (ii) hybridize the second probe sequence of the second probe to the second target region of the nucleic acid molecule(s) to yield a probe-associated nucleic acid molecule(s) comprising the nucleic acid molecule(s) hybridized to the first probe and the second probe; (c) subjecting the probe-associated nucleic acid molecule(s) to conditions sufficient to link the first probe to the second probe, thereby generating an additional nucleic acid molecule(s) comprising the first probe linked to the second probe; and (d) using the additional nucleic acid molecule(s) and a polynucleotide that comprises a second barcode sequence to generate a barcoded nucleic acid molecule(s) comprising (i) a sequence corresponding to the first target region and the second target region, (ii) the first barcode sequence or reverse complement thereof, and (iii) the second barcode sequence or reverse complement thereof.

In some aspects, the nucleic acid molecules are DNA. In some aspects, the nucleic acid molecules are RNA. In some aspects, the RNA are mRNA.

In some aspects, the method is drawn to utilizing a plurality of first and second probes that hybridize to a plurality of first and second target regions. In some aspects, the method is drawn to utilizing a sufficient number of first and second probe pairs such that substantially all of the gene-encoding RNA sequences, such as mRNAs, hybridize to the first and second probe pairs. In some aspects, the method is drawn to utilizing a sufficient number of first and second probe pairs such that at least about 50%, at least about 60%, at least about 70%, at least about 80%, at least about 90%, or at least about 95% of all of the gene-encoding RNA sequences, such as mRNAs, hybridize to the first and second probe pairs.

In some aspects, the sample comprises one or more cells, wherein the nucleic acid molecules are contained with the cell. In some aspects, the cells are prokaryotic cells or eukaryotic cells. In some aspects, the cells are animal cells, mammalian cells, insect cells, plant cells, or fungal cells. In some aspects, the cells are human cells. In some aspects, the cells are blood cells, immunological cells, neurological cells, muscle cells, skin cells, liver cells, lung cells, testis cells, ovarian cells, intestinal cells, pancreatic cells, kidney cells, cardiac cells, cancer cells, or tumor cells, or solid tumor cells.

In some aspects, the cells are fixed. In some aspects, the cell are not fixed or un-fixed. In some aspects, fixing cells comprised crosslinking components of the cells. In some aspects, the cells are fixed with an aldehyde fixative, a coagulant, an oxidizing agent, a simple fixative, a compound fixative, a histochemical fixative, a microanatomical fixative, a vapor fixative, or a cytological fixative. In some aspects, the cells are fixed with formaldehyde, paraformaldehyde, acrolein, glutaraldehyde, osmium tetroxide, picric acid, mercuric chloride, formalin, an alcohol, methyl alcohol, ethyl alcohol, acetic acid, glacial acetic acid, Bouin's fluid, formol saline, Zenker's fluid, formol calcium, Heidenhain's susa, Helly's fluid, Rossman's fluid, Champy's fluid, Carnoy's fluid, Clarke's fluid, Newcomer's fluid, or Flemming's fluid.

A sample (e.g., a cell sample) may be subjected to a fixation process at any useful point in time. For example, cells, nuclei and/or cellular/nuclear constituents of a sample may be subjected to a fixation process involving one or more fixation agents (e.g., as described herein) prior to commencement of any subsequent processing, such as for storage. Cells, nuclei and/or cellular/nuclear constituents, such as cells, nuclei and/or cellular/nuclear constituents of a tissue sample, subjected to a fixation process prior to storage, may be stored in an aqueous solution, optionally in combination with one or more preserving agents configured to preserve morphology, size, or other features of the cells and/or cellular components. Fixed cells, nuclei and/or cellular/nuclear constituents may be stored below room temperature, such as in a freezer. Alternatively, cells, nuclei and/or cellular/nuclear constituents of a sample may be subjected to a fixation process involving one or more fixation agents subsequent to one or more other processes, such as filtration, centrifugation, agitation, selective precipitation, purification, permeabilization, isolation, heating, etc. For example, cells, nuclei, and/or cellular/nuclear constituents of a given type from a sample may be subjected to a fixation process following a separation and/or enrichment procedure (e.g., as described herein). In an example, a sample comprising a plurality of cells including a plurality of cells of a given type may be subjected to a positive separation process to provide a sample enriched in the plurality of cells of the given type. The enriched sample may then be subjected to a fixation process involving one or more fixation agents (e.g., as described herein) to provide an enriched sample comprising a plurality of fixed cells. A fixation process may be performed in a bulk solution. In some aspects, fixed samples (e.g., fixed cells, fixed nuclei, and/or cellular/nuclear constituents) may be partitioned amongst a plurality of partitions (e.g., droplets or wells) and subjected to processing as described elsewhere herein. In some aspects, fixed samples may undergo additional processing, such as partial or complete reversal of a fixation process by, for example, rehydration or de-crosslinking, prior to partitioning and any subsequent processing. In some aspects, fixed samples may undergo partial or complete reversal of a fixation process within a plurality of partitions (e.g., prior to or concurrent with additional processing described elsewhere herein).

In some aspects, the methods further comprise lysing or permeabilizing the cells, thereby providing access to the nucleic acid molecules.

In some aspects, the fixed cells are a suspension of cells. In some aspects, the fixed cells are formalin-fixed paraffin-embedded (FFPE) cells. In some aspects, the fixed cells are embedded in an embedding medium. In some aspects, the embedding medium is selected from a wax, paraffin, optimal cutting temperature (OCT), resin, a polymer, or a plastic.

In some aspects, the methods of analyzing samples are performed on multiple samples in distinct reactions, wherein the barcode first barcode in a first sample is not the same as the first barcode in the second sample, thus allowing for the samples to be pooled together or multiplexed. In some aspects, the multiplexed samples comprise at least 2, at least 3, at least 4, at least 5, at least 6, at least 7, at least 8, at least 9, at least 10, at least 11, at least 12, at least 13, at least 14, at least 15, at least 20 or at least 30 different samples; wherein each sample comprises a different first barcode.

In some aspects, the embodiment comprising using the additional nucleic acid molecule and a polynucleotide that comprises a second barcode sequence to generate a barcoded nucleic acid molecule—the embodiment is performed in a partition, such as a well among a plurality of wells, a microwell among a plurality of microwells, and a droplet among a plurality of droplets.

In some aspects, the first probe and the second probe are linked together via ligation.

In some aspects, the nucleic acid barcode molecule is coupled to a support. In some aspects, the support is a bead, such as a gel bead or a glass bead. In some aspects, the nucleic acid barcode molecule is coupled to the support via a labile moiety, which may be thermolabile, photocleavable, or enzymatically cleavable.

### Preparing and Utilizing FFPE Tissue for Fixed RNA Profiling

In some aspects, a tissue specimen comprising a plurality of cells, nuclei and/or cellular/nuclear constituents may be processed to provide formalin-fixed paraffin-embedded (FFPE) tissue. A tissue specimen may be contacted (e.g., saturated) with formalin and then embedded in paraffin wax. FFPE processing may facilitate preservation of a tissue sample (e.g., prior to subsequent processing and analysis). A tissue sample, including an FFPE tissue sample, may additionally or alternatively be subjected to storage in a low-temperature freezer. Cells, nuclei and/or cellular/nuclear constituents may be dissociated from a tissue sample (e.g., FFPE tissue sample) prior to undergoing subsequent processing. In some aspects, individual cells, nuclei and/or cellular/nuclear constituents of a tissue sample such as an FFPE tissue sample may be optically detected, labeled, or otherwise processed prior to any such dissociation. Such detection, labeling, or other processing may be performed according to a 2- or 3-dimensional array and optionally according to a pre-determined pattern. In some aspects, a tissue specimen may be embedded in other materials such as, for example, optimal cutting temperature (OCT) compound, crosslinking-based supports (e.g., polymers), or the like.

The tissue specimen may have been fixed at least about 1 day (d), 2 d, 3 d, 4 d, 5 d, 6 d, 1 week (wk), 2 wk, 3 wk, 1 month (m), 2 m, 3 m, 4 m, 5 m, 6 m, 7 m, 8 m, 9 m, 10 m, 11 m, 1 year (y), 2 y, 3 y, 4 y, 5 y, 6 y, 7 y, 8 y, 9 y, 10 y, 15 y, 20 y, 25 y, 30 y, 35 y, 40 y, 45 y, 50 y, or longer before use in the methods and systems described elsewhere herein.

In some aspects, preparation of a fixed sample may comprise securing and sectioning at least a portion of a fixed sample (e.g., via microtomy, ultramicrotomy, etc.). The fixed sample may be sectioned into one or more (e.g., a plurality) of scrolls. A scroll may be at least about 1, 5, 10, 20, 30, 40, 50, 60, 70, 80, 90, 100, or more micrometers in thickness. The sectioning and/or securing may be performed at ambient temperature. The sectioning and/or securing may be performed at temperatures above or below ambient temperature.

In some aspects, the scroll can be mechanically and/or enzymatically dissociated. The mechanical dissociation may comprise sonication (e.g., sonication at below ambient temperatures). The sonication may comprise a sonication at a power of at least about 5, 10, 15, 20, 25, 30, 35, 40, 45, 50, 55, 60, 65, 70, 65, 80, 85, 90, 95, or 100 percent power. The sonication may comprise sonication at a power of at most about 100, 95, 90, 85, 80, 75, 70, 65, 60, 55, 50, 45, 40, 35, 30, 25, 20, 15, 10, 5, or less percent power. The sonication may be for at least about 1, 2, 3, 4, 5, 6, 7, 8, 9, 10, 15, 30, 45, 60, or more minutes. The sonication may be for at most about 60, 45, 30, 15, 10, 9, 8, 7, 6, 5, 4, 3, 2, 1, or fewer minutes. The mechanical dissociation may comprise use of a shake plate. For example, the sample can be placed in a sample tube, and the sample tube can be shaken on a shake plate. The mechanical dissociation may comprise stirring the sample.

In some optional aspects, the fixed sample may be processed to remove one or more fixatives and/or supports. For example, the fixed sample can be deparaffinized. In some aspects, the fixed sample may not be processed to remove the one or more fixatives and/or supports. For example, a fixed sample can be used as sectioned. Examples of processes include use of one or more non-polar solvents (e.g., linear alkanes, cyclic alkanes, benzene, xylenes, neo-clear, orange oil, other substituted or non-substituted alkanes, or the like, or any combination thereof). The removal of the one or more fixatives and/or supports may be repeated (e.g., for more complete removal of the one or more fixatives and/or supports). In some optional aspects, the sample may be rehydrated (e.g., via water addition, ethanol rehydration, gaseous rehydration, or the like, or any combination thereof). For example, ethanolic solutions of water with increasing water concentration can be used to rehydrate the sample. In some aspects, the sample can be analyzed without rehydration. The sample may be washed using a polar (e.g., aqueous) solution to remove additional impurities. For example, an aqueous solution of phosphate buffered saline can be used to remove impurities from a sample.

One or more dissociation solutions comprising, for example, liberase with low thermolysis (TL), liberase with medium thermolysis (TM), liberase with high thermolysis (TH), collagenases, or the like, or any combination thereof may be added to process a sample. The dissociation sample may be added at ambient (e.g., room) temperature. The dissociation solution be heated prior to the addition. The dissociation solution may be cooled prior to the addition. The dissociation sample may be at a temperature of at least about 20, 21, 22, 23, 24, 25, 26, 27, 28, 29, 30, 31, 32, 33, 34, 34, 36, 37, 38, 39, 40, or more degrees Celsius when added. The dissociation solution may be at a temperature of at most about 40, 39, 38, 37, 36, 35, 34, 33, 32, 31, 30, 29, 28, 27, 26, 25, 24, 23, 22, 21, or fewer degrees Celsius when added. The dissociation solution may be added at a temperature in a range as defined by any two of the proceeding values. The sample may be titrated at least about 5, 10, 15, 20, 25, 30, 35, 40, 45, 50, or more times to form a cellular suspension. In some aspects, impurities (e.g., paraffin) can be removed by allowing the suspension to rest, and the impurities can precipitate from the solution, and the purified solution can be removed and further processed.

The sample may be filtered one or more (e.g., 1, 2, 3, 4, 5, 6, 7, 8, 9, 10, or more) times. The filtration may comprise use of one or more different sizes (e.g., pore sizes) of filter. The filter may comprise a pore size of at most about 500, 400, 300, 200, 100, 90, 80, 70, 60, 50, 40, 30, 20, 10, 5, 1, or less micrometers. For example, a first filtration with a 70 micrometer pore size filter can be performed. In this example, a second filtration with a 30 micrometer filtration can be performed, which can reduce debris (e.g., undissolved paraffin or other support) without reducing cellular recovery from the sample. The filtrate and the cellular suspension may be combined and subsequently centrifuged. The centrifugation may occur at a value of at least about 100, 200, 300, 400, 500, 600, 700, 850, 900, 950, 1,000, 1,100, 1,200, 1,300, 1,400, 1,500, 1,600, 1,700, 1,800, 1,900, 2,000, 2,100, 2,200, 2,300, 2,400, 2,500, 3,000, or more reciprocal centrifugal force (rcf). The centrifugation may occur at a value of at most about 3,000, 2,500, 2,400, 2,300, 2,200, 2,100, 2,000, 1,900, 1,800, 1,700, 1,600, 1,500, 1,400, 1,300, 1,200, 1,100, 1,000, 950, 900, 850, 800, 700, 600, 500, 400, 300, 200, 100, or fewer rcf. The centrifugation may be performed at a value within a range as defined by any two of the proceeding values. For example, the centrifugation may be performed at a value of about 850 to about 2,000 rcf.

In some aspects, the solution can be removed from the centrifuged pellet (e.g., without disturbing the pellet). In some aspects, the pellet can be resuspended into solution. For example, the pellet can be resuspended into a buffer solution.

The resuspended solution can then be analyzed (e.g., to determine cellular concentration). Examples of cellular concentration determination systems include, but are not limited to the Countess II FL Automated Cell Counter, Cellaca MX High-Throughput Automated Cell Counter, or the like using a fluorescent dye (e.g., ethidium homodimer-1, ect.) or AO/PI staining solution, or the like. The resuspended solution may then be used as a sample for the methods and systems described elsewhere herein (e.g., RNA profiling, etc.).

In some aspects, use of a fixed sample (e.g., an FFPE sample) with the methods and systems described elsewhere herein may provide different information as compared to use of a fresh sample. For example, the fixation process may capture ephemeral states of the cells of the sample and/or ephemeral types of cells in the sample that may not be captured in fresh samples. In this way, cellular processes can be investigated in different ways by use of fixed samples. In some aspects, use of the methods and systems described elsewhere herein on fixed samples may provide unexpected improvements to the analysis of the fixed samples, such as improved sensitivity versus other analysis methods as well as the aforementioned analysis of different ephemeral states/types within the sample. In some aspects, the use of fixed samples may permit time series analysis of samples (e.g., samples can be fixed at different times and later analyzed). Such a time series analysis may provide information related to the evolution of states and/or cell types within the samples.

f. Spatial Array Analysis

A fixed biological sample disclosed herein can be used for spatial array analysis after assessing the quality (e.g., level of fixation) of the sample. The fixed biological sample can be de-crosslinked or additionally fixed before contacting with nucleic acid probes for spatial array analysis.

In some embodiments, provided herein is a method for sample processing and/or analysis, comprising: a) contacting a fixed biological sample with a nucleic acid stain and/or an actin stain; b) detecting an optical signal associated with the nucleic acid stain and/or an optical signal associated with the actin stain in the fixed biological sample; and c) comparing the optical signal(s) detected in b) to a reference to determine the quality of the sample, followed by spatial array analysis of the biological sample. In some embodiments, the spatial array analysis comprises d) transferring an analyte or corresponding probe or ligated probe set from the biological sample to an array of features on a substrate, each of which comprises a spatial barcode sequence associated with a unique spatial location on the array; e) generating a nucleic acid molecule comprises i) the spatial barcode sequence or a complement thereof and ii) a sequence of the analyte or corresponding probe or ligated probe set, or a complement thereof, and f) determining a sequence of the nucleic acid molecule, thereby determining the spatial location of the analyte or corresponding probe or ligated probe set in the biological sample.

In some embodiments, the method comprises contacting the biological sample with the probe corresponding to an analyte or with a probe set corresponding to the analyte (i.e., a probe or probe set for detecting the analyte), wherein the probe or probe set hybridizes to the analyte in the biological sample. In some embodiments, the method comprises contacting the biological sample with a probe set corresponding to the analyte and ligating the probe set to generate the ligated probe set. In some embodiments, the fixed biological sample is de-crosslinked or additionally fixed before contacting with the probe or probe set.

In some embodiments, after assessing the quality (e.g., level of fixation) of the sample, the assay may further comprise one or more steps for transferring probes for detecting an analyte (or a product or derivative thereof) to an array for spatial assay (e.g., performing NGS sequencing to determine one or more sequences of the oligonucleotides captured on the array). In some embodiments, a probe or probe set for detecting an analyte can be ligated in the biological sample and transferred to an array. In some embodiments, a product (e.g., extension product) or derivative of the ligated probes can be transferred to an array.

In one aspect, provided herein are methods, compositions, apparatus, and systems for spatial analysis of a biological sample, for example, a spatial array-based analysis. Non-limiting aspects of spatial analysis methodologies are described in U.S. Pat. Nos. 10,308,982; 9,879,313; 9,868,979; Liu et al., bioRxiv 788992, 2020; U.S. Pat. Nos. 10,774,372; 10,774,374; WO 2018/091676; U.S. Pat. Nos. 10,030,261; 9,593,365; 10,002,316; 9,727,810; 10,640,816; Rodriques et al., Science 363(6434):1463-1467, 2019; U.S. Pat. No. 11,447,807; Lee et al., Nat. Protoc. 10(3):442-458, 2015; U.S. Pat. Nos. 10,179,932; 11,085,072; 10,138,509; Trejo et al., PLoS ONE 14(2):e0212031, 2019; U.S. Patent Application Publication No. 2018/0245142; Chen et al., Science 348(6233):aaa6090, 2015; Gao et al., BMC Biol. 15:50, 2017; WO 2017/144338; US 2018/0372736; US 2022/0290228; U.S. Pat. No. 11,597,965; WO 2011/094669; U.S. Pat. Nos. 7,709,198; 8,604,182; 8,951,726; 9,783,841; 10,041,949; WO 2016/057552; US 2021/0238665; U.S. Pat. Nos. 10,370,698; 10,724,078; 10,364,457; 10,317,321; US 2021/0395796; US 2020/0239946; U.S. Pat. Nos. 10,059,990; 11,505,819; 11,104,936; and Gupta et al., Nature Biotechnol. 36:1197-1202, 2018, all of which are herein incorporated by reference in their entireties, and can be used herein in any combination. Further non-limiting aspects of spatial analysis methodologies are described herein.

In some aspects, the biological sample is on the substrate (e.g., a cover slip with sufficient strength comprising the capture array). In some embodiments, the biological sample is on a second substrate. The biological sample may be positioned between the first substrate (e.g., substrate comprising the capture array) and the second substrate (e.g., slide comprising the biological sample) such that the capture agents are allowed to capture the reporter oligonucleotides or derivatives thereof. In some embodiments, the biological sample is processed to release the probes or products thereof. The permeabilization step (e.g., using Proteinase K) allows the ligated probes to migrate onto the capture array substrate and be captured by the capture probes. In some aspects, the permeabilization may be combined with lysing.

In some embodiments, a method disclosed herein comprises transferring one or more analytes or corresponding probes or products thereof from a biological sample to an array of features on a substrate, each of which is associated with a unique spatial location on the array. Each feature may comprise a plurality of capture agents capable of capturing one or more nucleic acid molecules, and each of the capture agents of the same feature may comprise a spatial barcode corresponding to a unique spatial location of the feature on the array. In some embodiments, the method comprises capturing the analytes or corresponding probes or products thereof by a capture agent (e.g., capture probe). The capture probe comprises a capture domain that binds to a capture region on the reporter oligonucleotide or a complement thereof. One or more reactions (e.g., extension, and/or ligation) are performed to generate a spatially labeled polynucleotide sequence comprising (i) a sequence of the captured reporter oligonucleotide or a complement thereof, and (ii) a sequence of the spatial barcode (e.g., spatial barcode of the capture probe) or complement thereof. Subsequent analysis of the transferred analytes includes determining the identity of the analytes and the spatial location of each analyte within the sample. The spatially labeled polynucleotide or a portion thereof may be removed from the substrate (e.g., capture array) for sequencing using any suitable nucleic acid sequencing techniques, including next-generation sequencing (NGS). In some embodiments, the sequence of the spatially labeled polynucleotide is determined to detect the spatial barcode and the reporter oligonucleotide. All or part of the sequence of the generated spatially labeled polynucleotide may be determined. The spatial location of each analyte (e.g., reporter oligonucleotide or complement thereof) within the sample is determined based on the feature to which each analyte is bound in the array, and the feature's relative spatial location within the array.

In some embodiments, a method disclosed herein comprises associating a spatial barcode with one or more analytes (e.g., or corresponding probes or products thereof), such that the spatial barcode identifies the one or more analytes, and/or contents of the one or more cells, as associated with a particular spatial location.

In some embodiments, a method disclosed herein comprises driving target analytes out of a cell and towards a spatially-barcoded array. The target analytes (e.g., or corresponding probes or products thereof), interact with capture probes on the spatially-barcoded array. Once the target analyte is bound (e.g., hybridizes) to the capture probe, the sample is optionally removed from the array and the capture probes are analyzed in order to obtain spatially-resolved analyte information.

In some embodiments, a method disclosed herein comprises delivering or driving spatially-barcoded nucleic acid molecules (e.g., capture probes) towards and/or into or onto a sample. In some embodiments, a method disclosed herein comprises cleaving spatially-barcoded nucleic acid molecules (e.g., capture probes) from an array and driving the cleaved nucleic acid molecules towards and/or into or onto a sample. Alternatively, the sample may be permeabilized and fixed/crosslinked to restrict mobility of one or more target analytes, while allowing spatially-barcoded capture probes to migrate towards and/or into or onto the sample. Once the spatially-barcoded capture probe is associated with a particular analyte, the sample can be optionally removed for analysis. The sample can be optionally dissociated before analysis. Once the tagged analyte or cell is associated with the spatially-barcoded capture probe, the capture probes can be analyzed to obtain spatially-resolved information about the tagged analyte or cell.

A wide variety of different sequencing methods can be used to analyze spatially barcoded analyte constructs. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various commercial systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification.

Other examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and any combinations thereof.

In some embodiments, direct sequencing of one or more captured analytes is performed by sequencing-by-synthesis (SBS). In some embodiments, a sequencing primer is complementary to a sequence in one or more of the domains of a capture probe (e.g., functional domain). In such embodiments, sequencing-by-synthesis can include reverse transcription and/or amplification in order to generate a template sequence (e.g., functional domain) from which a primer sequence can bind.

SBS can involve hybridizing an appropriate primer, sometimes referred to as a sequencing primer, with the nucleic acid template to be sequenced, extending the primer, and detecting the nucleotides used to extend the primer. Preferably, the nucleic acid used to extend the primer is detected before a further nucleotide is added to the growing nucleic acid chain, thus allowing base-by-base nucleic acid sequencing. The detection of incorporated nucleotides is facilitated by including one or more labelled nucleotides in the primer extension reaction. To allow the hybridization of an appropriate sequencing primer to the nucleic acid template to be sequenced, the nucleic acid template should normally be in a single stranded form. If the nucleic acid templates making up the nucleic acid spots are present in a double stranded form these can be processed to provide single stranded nucleic acid templates using any suitable methods, for example by denaturation, cleavage etc. The sequencing primers which are hybridized to the nucleic acid template and used for primer extension are preferably short oligonucleotides, for example, 15 to 25 nucleotides in length. The sequencing primers can be provided in solution or in an immobilized form. Once the sequencing primer has been annealed to the nucleic acid template to be sequenced by subjecting the nucleic acid template and sequencing primer to appropriate conditions, primer extension is carried out, for example using a nucleic acid polymerase and a supply of nucleotides, at least some of which are provided in a labelled form, and conditions suitable for primer extension if a suitable nucleotide is provided.

In some cases, the assessment of the samples described herein (e.g., in Section III) can provide a quality map for the sample prior to transfer of the analytes (or probes or products thereof) to the spatial array. In some cases, the quality map can be used to select regions of interest, to screen samples prior to the analytes (e.g., or corresponding probes or products thereof) interacting with capture probes on the spatially-barcoded array, and/or sequencing the spatially barcoded analyte constructs. In some aspects, assessment of the samples described herein can be used to remove data from certain identified regions of low quality in the sample during or after analysis (e.g., sequencing).

### V. Samples and Sample Processing

A sample disclosed herein can be or derived from any biological sample. Methods, probes, and kits disclosed herein may be used for analyzing a biological sample, which may be obtained from a subject using any of a variety of techniques including, but not limited to, biopsy, surgery, and laser capture microscopy (LCM), and generally comprises cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be obtained as a tissue sample, such as a tissue section, a portion of a cell block or cell pellet, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, the biological sample comprises a tissue sample. In some instances, the tissue sample is a tissue biopsy. In some instances, the biological sample is a tumor biopsy. In some instances, the biological sample is a surgical resection. In some instances, the biological sample comprises a tumor or a portion of a tumor.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (iv) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using H&E staining techniques, Papanicolaou staining techniques, Masson's trichrome staining techniques, silver staining techniques, Sudan staining techniques, and/or Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain. In some embodiments, a fixed biological sample disclosed herein can be stained using H&E to detect under-fixation. For instance, an under-fixed cell or tissue sample stained with H&E can show broken nucleus envelop and sometimes lighter staining. In some embodiments, a fixed biological sample disclosed herein assessed for quality (e.g., as described in Section V) can be additionally stained using H&E after the assessment.

In some embodiments, biological samples can be destained. Methods of destaining or discoloring a biological sample generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (v) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

### (vi) Tissue Permeabilization and Treatment

In some embodiments, a biological sample can be permeabilized to facilitate transfer of species (such as probes) into the sample. If a sample is not permeabilized sufficiently, probes that enter the sample and bind to analytes therein may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods that can be used herein include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

(vii) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

### IX. Compositions and Kits

In some embodiments, provided herein are kits, for example comprising a compound disclosed herein for staining a fixed biological sample, e.g., a nucleic acid stain and/or an actin stain. In some embodiments, provided herein are kits for assessing biological sample quality, for example comprising a compound disclosed herein for staining a fixed biological sample, e.g., a nucleic acid stain and/or an actin stain. In some embodiments, the compound is provided in a composition (e.g., a composition comprising DMSO) or kit and the kit can further comprise one or more other compositions, e.g., a buffer for the compound. The kit can comprise one or more reagents required for one or more steps comprising hybridization, ligation, extension, detection, and/or sample preparation as described herein. In some embodiments, the kit comprises one or more labelling agents, e.g., disclosed in Section VII. In some embodiments, the kit comprises one or more oligonucleotides, e.g., nucleic acid probes disclosed in Section VII, for detecting one or more nucleic acid analytes and/or one or more non-nucleic acid analytes. In some embodiments, the kit comprises one or more antibodies (e.g., for detecting protein analytes) which can be optionally labelled with a detectable label such as a fluorophore and/or a reporter oligonucleotide.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing (e.g., crosslinking), embedding, and/or permeabilizing the biological sample. In some embodiments, the kits contain reagents, such as enzymes and buffers. In some aspects, the kit can also comprise any of the reagents described herein, e.g., wash buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, and reagents for additional assays.

### X. Applications

In some aspects, the provided embodiments can be applied in an in situ method of analyzing target nucleic acids, or in a single cell method of analyzing target nucleic acids. In some embodiments, the target nucleic acids are RNAs.

In some embodiments, the target nucleic acid comprises a single-nucleotide polymorphism (SNP). In some embodiments, the target nucleic acid comprises is a single-nucleotide variant (SNV). In some embodiments, the target nucleic acid comprises a single-nucleotide substitution. In some embodiments, the target nucleic acid comprises a point mutation. In some embodiments, the target nucleic acid comprises a single-nucleotide insertion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### XI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“Multiplexing” or “multiplex assay” herein may refer to an assay or other analytical method in which the presence and/or amount of multiple targets, e.g., multiple nucleic acid target sequences, can be assayed simultaneously by using more than one probe, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### XII. Exemplary Embodiments

Among the embodiments provided herein are:

1. A method for sample processing and/or analysis, comprising:
a) contacting a fixed biological sample with a nucleic acid stain and/or an actin stain;
b) detecting an optical signal associated with the nucleic acid stain and/or an optical signal associated with the actin stain in the fixed biological sample; and
c) comparing the optical signal(s) detected in b) to a reference to determine the quality of the sample,
optionally wherein the method further comprises d1) de-crosslinking or additionally fixing the fixed biological sample to adjust the level of fixation, d2) contacting the fixed biological sample with a nucleic acid probe that directly or indirectly binds to an analyte or a product thereof in the fixed biological sample, or d3) adjusting the level of fixation of an additional biological sample and optionally contacting the additional biological sample with a nucleic acid probe that directly or indirectly binds to an analyte or a product thereof in the additional biological sample.
   2. The method of embodiment 1, further comprising:
d1) de-crosslinking or additionally fixing the fixed biological sample to adjust the level of fixation; and/or
d2) contacting the fixed biological sample with a nucleic acid probe that directly or indirectly binds to an analyte or a product thereof in the fixed biological sample.
   3. The method of embodiment 1, further comprising:
d3) adjusting the level of fixation of an additional biological sample, and optionally wherein the additional biological sample is contacted with a nucleic acid probe that directly or indirectly binds to an analyte or a product thereof in the additional biological sample.
   4. The method of any of embodiments 1-3, wherein the nucleic acid stain is cell-permeant.
   5. The method of any of embodiments 1-4, wherein the nucleic acid stain is non-fluorescent or substantially non-fluorescent in the absence of nucleic acids, and/or wherein the nucleic acid stain is fluorescent when bound to RNA.
   6. The method of any of embodiments 1-5, wherein the nucleic acid stain selectively binds to RNA over DNA.
   7. The method of any of embodiments 1-6, wherein the nucleic acid stain comprises a quinolinium scaffold and an aminoethylpiperidine group, optionally wherein the nucleic acid stain comprises (E)-2-(2-(1H-indol-3-yl)vinyl)-1-methylquinolin-1-ium iodide, (E)-2-(2-(1H-indol-2-yl)vinyl)-1-methyl-4-((2-(piperidin-1-yl)ethyl)amino) quinolin-1-ium iodide, or (E)-2-(2-(1H-indol-3-yl)vinyl)-1-methyl-4-((2-(piperidin-1-yl)ethyl)amino) quinolin-1-ium iodide.
   8. The method of any of embodiments 1-7, wherein the nucleic acid stain comprises DAPI, propidium iodide (PI), a Hoechst stain, and/or a fluorescent Nissl stain.
   9. The method of any of embodiments 1-7, wherein the nucleic acid stain does not comprise DAPI, propidium iodide (PI), a Hoechst stain, or a fluorescent Nissl stain.
   10. The method of any of embodiments 1-9, wherein the actin stain is fluorescent or conjugated to a fluorescent moiety.
   11. The method of any of embodiments 1-10, wherein the actin stain selectively binds to polymeric actin over monomeric actin.
   12. The method of any of embodiments 1-11, wherein the actin stain selectively binds to F-actin.
   13. The method of any of embodiments 1-12, wherein the actin stain comprises phalloidin or a derivative thereof.
   14. The method of any of embodiments 1-13, wherein the actin stain comprises an anti-actin antibody or an epitope-binding fragment thereof.
   15. The method of any of embodiments 1-14, wherein the fixed biological sample is contacted with the nucleic acid stain and the actin stain in a).
   16. The method of embodiment 15, wherein the fixed biological sample is contacted with the nucleic acid stain prior to, simultaneously with, or after contacting with the actin stain.
   17. The method of any of embodiments 1-16, wherein the fixed biological sample is fixed using a fixing composition which optionally comprises 0.01-100% of a fixative selected from the group consisting of: formaldehyde, glutaraldehyde, acetone, methanol, ethanol, acetic acid, potassium dichromate, chromic acid, potassium permanganate, B-5, Zenker's fixative, uranyl acetate, mercuric chloride, osmium tetroxide, potassium permanganate, and 1-ethyl-3-(3-dimethylamino propyl)carbodiimide (EDC), picric acid, glyoxal, bis(sulfosuccinimidyl)suberate, and derivatives thereof.
   18. The method of embodiment 17, wherein the fixing composition is free or substantially free of an alcohol.
   19. The method of embodiment 18, wherein the fixing composition is free or substantially free of methanol and ethanol.
   20. The method of any of embodiments 1-19, wherein the fixed biological sample is fixed using a neutral buffered formalin (NBF) or a paraformaldehyde (PFA) solution.
   21. The method of any of embodiments 1-20, wherein the fixed biological sample is immobilized on a substrate.
   22. The method of embodiment 21, wherein the substrate comprises a planar surface for sample contact prior to, during, and/or after fixing a biological sample to provide the fixed biological sample.
   23. The method of embodiment 21 or 22, wherein the substrate is a solid substrate and does not comprise a bead, particle, or microwell.
   24. The method of any of embodiments 21-23, wherein the substrate is transparent.
   25. The method of any of embodiments 21-24, wherein the substrate is a glass slide or a plastic slide.
   26. The method of any of embodiments 21-25, wherein the substrate does not comprise nucleic acid immobilized thereon prior to contacting the fixed biological sample.
   27. The method of any of embodiments 1-26, wherein the fixed biological sample is a tissue section, optionally wherein the tissue section is about 5 μm, about 10 μm, about 20 μm, about 30 μm, about 40 μm, or about 50 μm in thickness, optionally wherein the tissue section is a normal tissue section or associated with a disease or condition, and optionally wherein the tissue section comprises a cancer cell, a stem cell, an immune cell, an apoptotic cell, a necrotic cell, and/or a pathogen.
   28. The method of any of embodiments 1-27, wherein the fixed biological sample comprises dissociated cells, cultured cells, and/or cells isolated from a subject.
   29. The method of any of embodiments 1-28, wherein the fixed biological sample is a fresh frozen biological sample that has been fixed.
   30. The method of any of embodiments 1-28, wherein the fixed biological sample is a paraffin-embedded biological sample, optionally wherein the fixed biological sample is a formalin-fixed paraffin-embedded (FFPE) biological sample.
   31. The method of any of embodiments 1-30, wherein the fixed biological sample is de-paraffinizing prior to the contacting in a), optionally wherein the de-paraffinizing comprises contacting the fixed biological sample with xylene, ethanol, and water, or, sequentially contacting the fixed biological sample with xylene, absolute ethanol, about 96% ethanol, about 70% ethanol, and water.
   32. The method of any of embodiments 1-31, wherein the fixed biological sample is not de-crosslinked prior to or during the contacting in a).
   33. The method of any of embodiments 1-32, wherein the fixed biological sample is de-crosslinked after the comparing in c).
   34. The method of embodiment 32 or 33, wherein the de-crosslinking comprises contacting the fixed biological sample with a catalyst that catalyzes de-crosslinking of molecular crosslinks.
   35. The method of embodiment 34, wherein the catalyst non-enzymatically catalyzes de-crosslinking of inter-molecular crosslinks and/or intra-molecular crosslinks in the fixed biological sample, optionally wherein the inter-molecular crosslinks and/or intra-molecular crosslinks comprise an aminal bridge.
   36. The method of any of embodiments 1-32, wherein the fixed biological sample is additionally fixed after the comparing in c), optionally wherein the additional fixation comprises contacting the fixed biological sample with a crosslinking agent.
   37. The method of embodiment 36, wherein the fixed biological sample is additionally fixed using an additional fixing composition which optionally comprises 0.01-100% of a fixative selected from the group consisting of: formaldehyde, glutaraldehyde, acetone, methanol, ethanol, acetic acid, potassium dichromate, chromic acid, potassium permanganate, B-5, Zenker's fixative, uranyl acetate, mercuric chloride, osmium tetroxide, potassium permanganate, and 1-ethyl-3-(3-dimethylamino propyl)carbodiimide (EDC), picric acid, glyoxal, bis(sulfosuccinimidyl)suberate, and derivatives thereof.
   38. The method of embodiment 37, wherein the additional fixing composition comprises an alcohol, optionally wherein the alcohol is methanol or ethanol.
   39. The method of embodiment 37, wherein the additional fixing composition is free or substantially free of an alcohol, optionally wherein the additional fixing composition is a neutral buffered formalin (NBF) or a paraformaldehyde (PFA) solution.
   40. The method of any of embodiments 1-32, wherein the fixed biological sample is neither de-crosslinked nor additionally fixed after the comparing in c).
   41. The method of any of embodiments 1-40, wherein the nucleic acid stain is a first nucleic acid stain, and the detecting in b) comprises detecting an optical signal associated with a second nucleic acid stain.
   42. The method of embodiment 41, wherein the first nucleic acid stain selectively binds to RNA and the second nucleic acid stain selectively binds to DNA, optionally wherein the second nucleic acid stain is DAPI, propidium iodide (PI), a Hoechst stain, or a fluorescent Nissl stain.
   43. The method of embodiment 41 or 42, wherein the optical signal associated with the first nucleic acid stain and the optical signal associated with the second nucleic acid stain are detected in a nucleus.
   44. The method of any of embodiments 41-43, wherein the comparing in c) comprises using a ratio between the optical signal associated with the first nucleic acid stain and the optical signal associated with the second nucleic acid stain in the fixed biological sample.
   45. The method of any of embodiments 1-44, wherein the comparing in c) comprises using a ratio between the optical signal associated with the nucleic acid stain and an optical signal associated with background signal detected in a cytoplasm.
   46. The method of embodiment 45, wherein the nucleic acid stain selectively binds to RNA.
   47. The method of embodiment 45 or 46, wherein the optical signal associated with the nucleic acid stain is detected in a nucleus.
   48. The method of embodiment 47, wherein the fixed biological sample is contacted with an additional nucleic acid stain or a cytoplasm stain for determining the location of the nucleus.
   49. The method of embodiment 48, wherein the additional nucleic acid stain selectively binds to DNA.
   50. The method of embodiment 49, wherein the additional nucleic acid stain is DAPI.
   51. The method of any of embodiments 1-44, wherein the comparing in c) comprises using a ratio between the optical signal associated with the actin stain and an optical signal associated with an additional nucleic acid stain in the fixed biological sample.
   52. The method of embodiment 51, wherein the additional nucleic acid stain selectively binds to DNA.
   53. The method of embodiment 52, wherein the additional nucleic acid stain is DAPI.
   54. The method of any of embodiments 51-53, wherein the optical signal associated with the actin stain is detected in a cytoplasm and the optical signal associated with the additional nucleic acid stain is detected in a nucleus.
   55. The method of any of embodiments 1-54, wherein the comparing in c) comprises using an optical signal associated with the nucleic acid stain, an optical signal associated with the actin stain, an optical signal associated with an additional nucleic acid stain, and/or an optical signal associated with an additional actin stain in a reference sample.
   56. The method of embodiment 55, wherein an elevated level of the nucleic acid stain and/or the additional nucleic acid stain in the fixed biological sample compared to that in the reference sample indicates the fixed biological sample is less fixed than the reference sample, and/or wherein a decreased level of the nucleic acid stain and/or the additional nucleic acid stain in the fixed biological sample compared to that in the reference sample indicates the fixed biological sample is more fixed than the reference sample.
   57. The method of embodiment 55 or 56, wherein an elevated level of the actin stain and/or the additional actin stain in the fixed biological sample compared to that in the reference sample indicates the fixed biological sample is more fixed than the reference sample, and/or wherein a decreased level of the actin stain and/or the additional actin stain in the fixed biological sample compared to that in the reference sample indicates the fixed biological sample is less fixed than the reference sample.
   58. The method of any of embodiments 1-57, wherein:
i) the fixed biological sample is neither over-fixed nor under-fixed based on the comparing in c), and the method does not comprise de-crosslinking or additionally fixing the fixed biological sample;
ii) the fixed biological sample is over-fixed based on the comparing in c), and the method comprises de-crosslinking the fixed biological sample to provide a de-crosslinked fixed biological sample; or
iii) the fixed biological sample is under-fixed based on the comparing in c), and the method comprises additionally fixing the fixed biological sample to provide an additionally fixed biological sample.
   59. The method of embodiment 58, comprising permeabilizing the fixed biological sample that is neither de-crosslinked nor additionally fixed, the de-crosslinked fixed biological sample, or the additionally fixed biological sample.
   60. The method of embodiment 58 or 59, comprising contacting a cell or nucleus in the fixed biological sample that is neither de-crosslinked nor additionally fixed, the de-crosslinked fixed biological sample, or the additionally fixed biological sample with the nucleic acid probe that directly or indirectly binds to the analyte in the cell or nucleus.
   61. The method of embodiment 60, comprising detecting an optical signal associated with the nucleic acid probe or a product thereof at a location of the cell or nucleus, thereby detecting the analyte at the location in the fixed biological sample that is neither de-crosslinked nor additionally fixed, the de-crosslinked fixed biological sample, or the additionally fixed biological sample.
   62. The method of embodiment 60, comprising partitioning the cell or nucleus in a partition comprising a partition barcode, optionally wherein the partition is an emulsion droplet or a microwell.
   63. The method of embodiment 62, comprising sequencing a nucleic acid molecule or a portion thereof comprising i) a sequence of the nucleic acid probe or a complement thereof and ii) the partition barcode or a complement thereof.
   64. The method of any of embodiments 1-63, wherein after the comparing in c) and prior to d1) and/or d2), the nucleic acid stain and/or the actin stain are removed.
   65. The method of any of embodiments 1-63, wherein after the comparing in c) and prior to d1) and/or d2), the nucleic acid stain and/or the actin stain are not removed.
   66. The method of any of embodiments 1-65, wherein prior to, during, and/or after d1) and/or d2), the nucleic acid stain and/or the actin stain are removed by a buffer comprising a salt, a divalent cation, a denaturing agent, an ionic detergent, and/or a non-ionic detergent.
   67. The method of embodiment 66, wherein the nucleic acid stain and/or the actin stain are removed after d2) and the buffer comprises saline-sodium citrate (SSC) and/or formamide.
   68. The method of any of embodiments 1-67, comprising removing unbound and nonspecifically bound nucleic acid probe, wherein the nucleic acid stain and/or the actin stain are removed together with the unbound and nonspecifically bound nucleic acid probe.
   69. The method of any of embodiments 1-68, wherein the nucleic acid probe comprises a detectable label, optionally wherein the detectable label comprises a nucleic acid sequence or an optically detectable label.
   70. The method of any of embodiments 1-69, wherein the nucleic acid probe comprises a barcode region comprising one or more barcode sequences.
   71. The method of any of embodiments 1-70, wherein the analyte is a cellular nucleic acid, optionally wherein the cellular nucleic acid is genomic DNA, RNA, or cDNA.
   72. The method of embodiment 71, wherein the nucleic acid probe is a primary probe that hybridizes to the cellular nucleic acid.
   73. The method of embodiment 72, wherein the primary probe comprises a barcode sequence corresponding to the cellular nucleic acid or a portion thereof.
   74. The method of embodiment 72 or 73, wherein the primary probe is selected from the group consisting of: a primary probe comprising a 3′ or 5′ overhang upon hybridization to the cellular nucleic acid, optionally wherein the 3′ or 5′ overhang comprises one or more barcode sequences; a primary probe comprising a 3′ overhang and a 5′ overhang upon hybridization to the cellular nucleic acid, optionally wherein the 3′ overhang and the 5′ overhang each independently comprises one or more barcode sequences; a circular primary probe; a circularizable primary probe or probe set; a primary probe or probe set comprising a split hybridization region configured to hybridize to a splint, optionally wherein the split hybridization region comprises one or more barcode sequences; and a combination thereof.
   75. The method of any of embodiments 71-74, wherein the cellular nucleic acid is an mRNA, and a first nucleic acid probe and a second nucleic acid probe are hybridized to a first analyte sequence and a second analyte sequence in the mRNA, respectively, wherein:
the first nucleic acid probe comprises: i) a first hybridization region complementary to the first analyte sequence, and ii) a first overhang;
the second nucleic acid probe comprises: i) a second hybridization region complementary to the second analyte sequence, and ii) a second overhang;
the first and second nucleic acid probes are ligated using the mRNA as template to form a ligated nucleic acid probe, with or without gap filling prior to the ligation; and
the first and second overhangs independently comprise a primer binding sequence, a capture sequence, a barcode sequence, and/or a constant sequence.
   76. The method of embodiment 75, wherein the first overhang is a 5′ overhang comprising a primer binding sequence, and the second overhang is a 3′ overhang comprising a probe barcode sequence, a constant sequence, and a capture sequence on the 3′ end.
   77. The method of embodiment 76, wherein the capture sequence is complementary to a capture probe comprising a partition barcode, a primer binding sequence, and an UMI,
optionally wherein the capture probe is immobilized on a bead or a microwell of a partition,
optionally wherein upon hybridization of the capture sequence to the capture probe, the method comprises generating in the partition a nucleic acid molecule comprising the partition barcode, the UMI, a complement of the probe barcode sequence, the first analyte sequence, and the second analyte sequence.
   78. The method of embodiment 71, wherein nucleic acid probe is a detectable probe that hybridizes to a primary probe or a product or complex thereof, wherein the primary probe hybridizes to the cellular nucleic acid.
   79. The method of embodiment 78, wherein the product or complex of the primary probe is selected from the group consisting of:
a rolling circle amplification (RCA) product,
a complex comprising an initiator and an amplifier for hybridization chain reaction (HCR),
a complex comprising an initiator and an amplifier for linear oligonucleotide hybridization chain reaction (LO-HCR),
a primer exchange reaction (PER) product, and
a complex comprising a pre-amplifier and an amplifier for branched DNA (bDNA).
   80. The method of embodiment 78 or 79, wherein the detectable probe hybridizes to a barcode sequence in the primary probe or product or complex thereof.
   81. The method of any of embodiments 78-80, wherein the detectable probe comprises a barcode sequence in a region that does not hybridize to the primary probe or product or complex thereof.
   82. The method of any of embodiments 78-81, wherein the detectable probe is selected from the group consisting of: a detectable probe comprising a 3′ or 5′ overhang upon hybridization to the primary probe or product or complex thereof, optionally wherein the 3′ or 5′ overhang comprises one or more barcode sequences; a detectable probe comprising a 3′ overhang and a 5′ overhang upon hybridization to the primary probe or product or complex thereof, optionally wherein the 3′ overhang and the 5′ overhang each independently comprises one or more barcode sequences; a circular detectable probe; a circularizable detectable probe or probe set; a detectable probe or probe set comprising a split hybridization region configured to hybridize to a splint, optionally wherein the split hybridization region comprises one or more barcode sequences; and a combination thereof.
   83. The method of any of embodiments 78-82, wherein the detectable probe comprises a fluorescent label and/or a nucleic acid sequence for binding to a fluorescently labelled probe.
   84. The method of any of embodiments 1-83, wherein the analyte comprises a non-nucleic acid moiety, optionally wherein the non-nucleic acid moiety is a protein, a carbohydrate, a lipid, a small molecule, or a complex thereof.
   85. The method of embodiment 84, wherein the nucleic acid probe directly or indirectly binds to a reporter oligonucleotide conjugated to an analyte-binding moiety that directly or indirectly binds to the analyte, optionally wherein the analyte-binding moiety comprises an antibody or epitope binding fragment thereof or an aptamer.
   86. The method of any of embodiments 1-85, wherein the fixed biological sample in d1) and/or d2) is the same sample or a portion thereof as the fixed biological sample stained in a).
   87. The method of embodiment 86, wherein the fixed biological sample in d2), without or without having been de-crosslinked or additionally fixed in d1), is the same tissue section as the fixed biological sample stained in a)
   88. The method of embodiment 86, wherein the fixed biological sample in d2), without or without having been de-crosslinked or additionally fixed in d1), comprises dissociated cells or nuclei from the fixed biological sample stained in a).
   89. The method of any of embodiments 1-85, wherein the fixed biological sample in d1) and/or d2) and the fixed biological sample stained in a) are different portions of the same biological sample
   90. The method of embodiment 89, wherein the fixed biological sample in d 1) and/or d2) and the fixed biological sample stained in a) are consecutive sections of the same tissue sample.
   91. The method of embodiment 89, wherein a portion of the fixed biological sample is stained in a), and the fixed biological sample in d2), without or without having been de-crosslinked or additionally fixed in d1), comprises dissociated cells or nuclei from a portion that is different from the portion stained in a).
   92. A method for sample analysis, comprising:
a) contacting a fixed tissue section with a nucleic acid stain and/or an actin stain;
b) detecting an optical signal associated with the nucleic acid stain and/or an optical signal associated with the actin stain in the fixed tissue section;
c) comparing the optical signal(s) detected in b) to a reference;
d) contacting the fixed tissue section or a consecutive tissue section thereto in a fixed tissue sample with a nucleic acid probe that directly or indirectly binds to an analyte or a product thereof in the fixed tissue section or consecutive tissue section; and
e) detecting an optical signal associated with the nucleic acid probe or a product thereof at one or more locations in the fixed tissue section or consecutive tissue section, thereby detecting the analyte at the one or locations.
   93. The method of embodiment 92, wherein the fixed tissue sample is neither over-fixed nor under-fixed based on the comparing in c), and the method does not comprise de-crosslinking or additionally fixing the fixed tissue sample between c) and d).
   94. The method of embodiment 92, wherein the fixed tissue sample is over-fixed based on the comparing in c), and the method comprises:
between c) and d), de-crosslinking the fixed tissue section or consecutive tissue section,
in d), contacting the de-crosslinked fixed tissue section or consecutive tissue section with the nucleic acid probe, and
in e), detecting the optical signal in the de-crosslinked fixed tissue section or consecutive tissue section.
   95. The method of embodiment 92, wherein the fixed tissue sample is under-fixed based on the comparing in c), and the method comprises:
between c) and d), additionally fixing the fixed tissue section or consecutive tissue section,
in d), contacting the additionally fixed tissue section or consecutive tissue section with the nucleic acid probe, and
in e), detecting the optical signal in the additionally fixed tissue section or consecutive tissue section.
   96. The method of any of embodiments 92-95, wherein the method comprises:
contacting the fixed tissue section or consecutive tissue section with a first nucleic acid probe that directly or indirectly binds to a first analyte at a first location, and detecting a first optical signal associated with the first nucleic acid probe or a product thereof, and
contacting the fixed tissue section or consecutive tissue section with a second nucleic acid probe that directly or indirectly binds to a second analyte at a second location, and detecting a second optical signal associated with the second nucleic acid probe or a product thereof,
thereby detecting the first and second analytes at the first and second locations, respectively, in the fixed tissue section or consecutive tissue section.
   97. The method of embodiment 96, wherein the first and second analytes are cellular nucleic acid molecules, and optionally wherein the first and second analytes are mRNA molecules of the same gene or different genes.
   98. The method of embodiment 96, wherein the first analyte is a cellular nucleic acid molecule, and the second analyte is a protein, optionally wherein the first analyte is an mRNA molecule and the second analyte is an intracellular protein, a membrane-bound protein, or an extracellular protein.
   99. The method of any of embodiments 96-98, wherein the first and second locations are the same location or different locations.
   100. The method of any of embodiments 92-99, wherein the product of the nucleic acid probe is generated in situ
   101. The method of any of embodiments 92-100, wherein the optical signal is detected in situ.
   102. The method of any of embodiments 92-101, wherein the optical signal is detected by imaging the fixed tissue section or consecutive tissue section, optionally wherein the imaging comprises fluorescent microscopy.
   103. A method for sample analysis, comprising:
a) contacting a first portion of a fixed biological sample with a nucleic acid stain and/or an actin stain;
b) detecting an optical signal associated with the nucleic acid stain and/or an optical signal associated with the actin stain in the first portion of the fixed biological sample;
c) comparing the optical signal(s) detected in b) to a reference;
d) contacting dissociated cells or nuclei from the first portion and/or a second portion of the fixed biological sample with a nucleic acid probe that directly or indirectly binds to an analyte or a product thereof in the dissociated cells or nuclei;
e) partitioning the dissociated cells or nuclei in partitions, wherein a single-cell partition comprises one of the dissociated cells or nuclei and a partition barcode;
f) generating a nucleic acid molecule in the single-cell partition, wherein the nucleic acid molecule comprises i) a sequence of the nucleic acid probe or a complement thereof and ii) the partition barcode or a complement thereof, and
g) determining a sequence of the nucleic acid molecule, thereby detecting the analyte in one or more single cells from the fixed biological sample.
   104. The method of embodiment 103, wherein the first and second portions of the fixed biological sample are the same.
   105. The method of embodiment 103, wherein the first and second portions of the fixed biological sample are different.
   106. The method of any of embodiments 103-105, wherein the analyte is an mRNA, and a first nucleic acid probe and a second nucleic acid probe are hybridized to a first analyte sequence and a second analyte sequence in the mRNA, respectively, wherein:
the first nucleic acid probe comprises: i) a first hybridization region complementary to the first analyte sequence, and ii) a first overhang;
the second nucleic acid probe comprises: i) a second hybridization region complementary to the second analyte sequence, and ii) a second overhang; and
the first and second nucleic acid probes are ligated using the mRNA as template to form a ligated nucleic acid probe, with or without gap filling prior to the ligation.
   107. The method of embodiment 106, wherein:
the single-cell partition is an emulsion droplet or a microwell,
the nucleic acid molecule generated in the single-cell partition comprises the sequences of i) the ligated nucleic acid probe, ii) the partition barcode, and iii) a UMI, and
the nucleic acid molecules generated in a plurality of single-cell partitions are sequenced, thereby analyzing analytes in single cells from the fixed biological sample.
   108. The method of any of embodiments 103-107, wherein the fixed biological sample is neither over-fixed nor under-fixed based on the comparing in c), and the method does not comprise de-crosslinking or additionally fixing the fixed biological sample or the dissociated cells or nuclei between c) and d).
   109. The method of any of embodiments 103-107, wherein the fixed biological sample is over-fixed based on the comparing in c), and the method comprises:
between c) and d), de-crosslinking the fixed biological sample and/or the dissociated cells or nuclei, and
in d), contacting the de-crosslinked dissociated cells or nuclei with the nucleic acid probe.
   110. The method of any of embodiments 103-107, wherein the fixed biological sample is under-fixed based on the comparing in c), and the method comprises:
between c) and d), additionally fixing the fixed biological sample and/or the dissociated cells or nuclei, and
in d), contacting the additionally fixed dissociated cells or nuclei with the nucleic acid probe.
   111. The method of any of embodiments 103-110, wherein the fixed biological sample is a formalin-fixed paraffin-embedded (FFPE) biological sample.

### XIII. Examples

The following examples are included for illustrative purposes only and are not intended to limit the scope of the present disclosure.

### Example 1: Assessing Level of Fixation in Fixed Cells

SYTO™ RNASelect™ staining and phalloidin staining were used to assess level of fixation in fixed HEK293 cells. HEK293 adherent cells were plated. Cells were rinsed twice in Phosphate Buffered Saline (PBS), followed by fixation with 10% Neutral Buffered Formalin (NBF) for either 1 minute, 10 minutes, 60 minutes, or 180 minutes at room temperature (RT). Cells were then rinsed in PBS and blocked for 30 minutes at RT. The fixed cells were then incubated for 20 minutes at RT with a 1:250 dilution of phalloidin (e.g., phalloidin_Atto647N from Sigma-Aldrich) in blocking buffer and with 500 nM SYTO™ RNASelect™. The fixed cells were also stained with DAPI (5 pg/mL) for 1 minute.

Cells were imaged, and representative images are shown in FIG. 1A. FIG. 1B shows the phalloidin/DAPI signal ratio increasing with fixation time. While DAPI staining was relatively constant at all fixation times, RNASelect™ nuclear localization decreased as fixation time increased, and phalloidin staining increased with increased fixation time. Phalloidin signals can be normalized against DAPI signals in the same sample. As such, phalloidin and/or RNASelect™ staining can be used to detect over-fixation, where increased phalloidin staining signals can indicate over-fixation and concentration of RNASelect™ staining in the nucleus can indicate the sample is not over-fixed. Over-fixation can also be detected using a combination of phalloidin and RNASelect™ staining, where the sample is likely over-fixed when there are fewer cells with RNASelect™ staining in the nuclei and there is more phalloidin staining (e.g., as normalized to DAPI staining) in the sample.

### Example 2: Assessing Quality of Mouse FFPE Samples

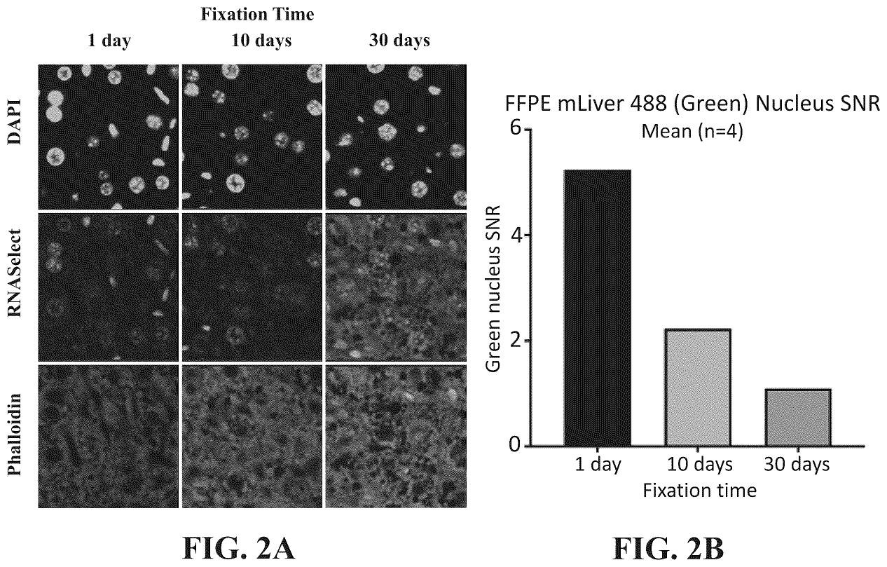

Mouse FFPE tissue samples were fixed for various times and cut into tissue sections of approximately 5 μm in thickness and placed on slides. The slides were baked at 60° C. and then de-paraffinized using xylene and absolute ethanol, and then re-hydrated using 70% ethanol (or an ethanol series of 95% ethanol followed by 70% ethanol) and nuclease free water (e.g., DEPC water). The de-paraffinized tissue sections were blocked for 30 minutes at room temperature (RT) and then stained with phalloidin in blocking buffer (e.g., 1:250 or 1:200 dilution) and with SYTO™ RNASelect™ (e.g., 250 nM or 500 nM). The tissue sections were also stained with DAPI (5 pg/mL) for 1 minute. The tissue sections were imaged using fluorescent microscopy. Mouse FFPE tissue samples tested included samples from mouse liver (FIGS. 2A-2B), placenta (data not shown), kidney (data not shown), lung (data not shown), and spleen (data not shown).

FIG. 2A shows representative images of mouse liver (mLiver) FFPE tissue sections fixed for 1 day, 10 days, or 30 days. The RNASelect™ nucleus signal-to-noise ratios (SNRs) are plotted against the fixation time in FIG. 2B. The RNASelect™ nucleus SNR was calculated as the ratio between signal intensity in a nucleus and signal intensity of a region around the nucleus. Nuclear localization of RNASelect™ staining and/or intensity of nuclear RNASelect™ signals were high in less fixed samples (e.g., 1-day fixation) and low in over-fixed samples (e.g., 10-day or 30-day fixation). Consistent with the results in fixed cells in Example 1, the results in this example show that phalloidin staining (e.g., ratio of phalloidin/DAPI intensity) in mouse FFPE samples increases as fixation time increases and the sample is over-fixed, whereas RNASelect™ staining is more concentrated in the nucleus (e.g., RNASelect™ nucleus SNR is higher) when the sample is not over-fixed.

### Example 3: Assessing Quality of Human FFPE Samples

Human FFPE tissue samples were fixed for various times and cut into tissue sections of approximately 5 μm in thickness and placed on slides. The slides were baked at 60° C. and then de-paraffinized using xylene and absolute ethanol/methanol, and then re-hydrated using 70% ethanol/methanol (or an ethanol/methanol series of 95% ethanol/methanol followed by 70% ethanol/methanol) and nuclease free water (e.g., DEPC water).

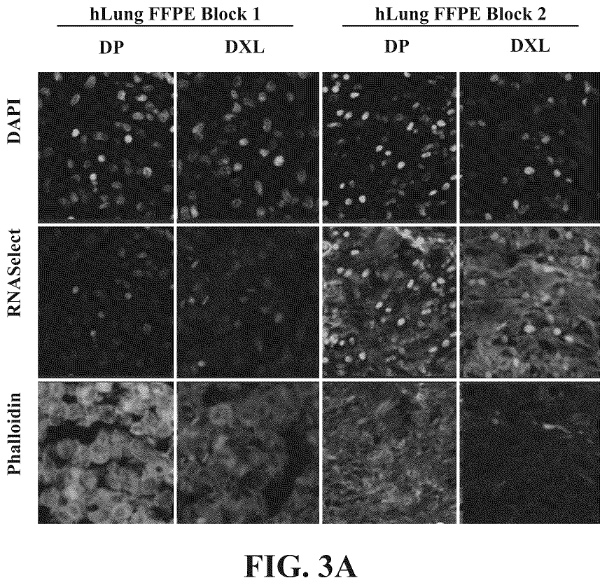

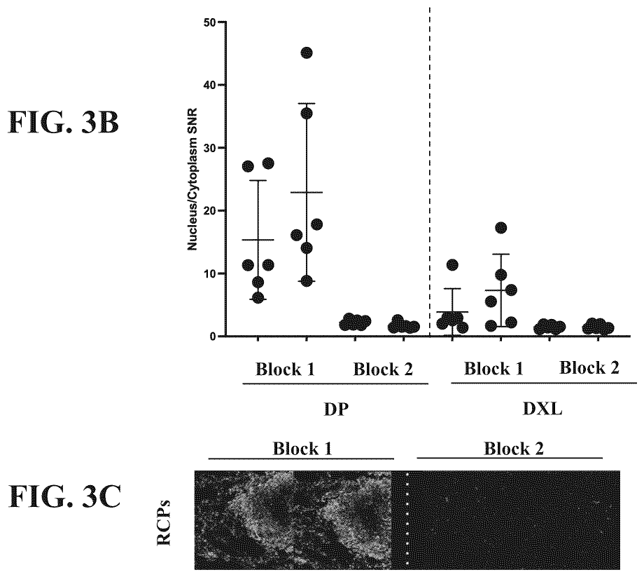

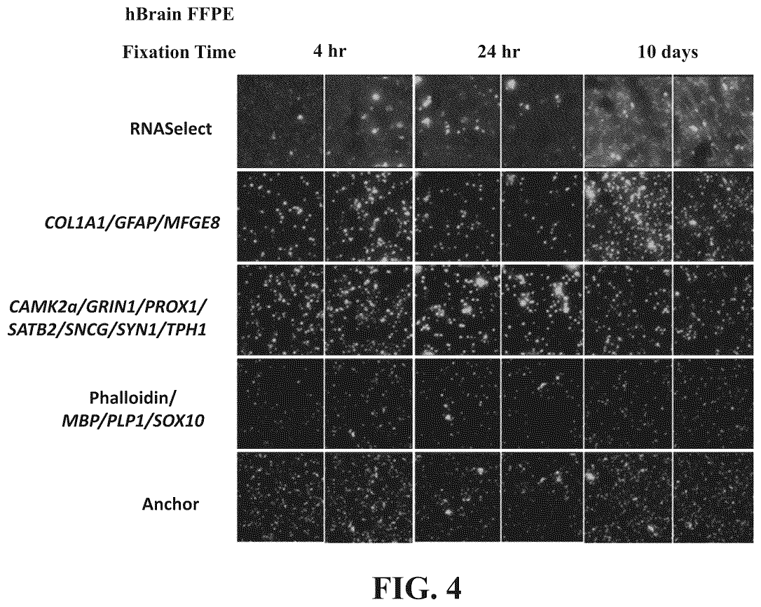

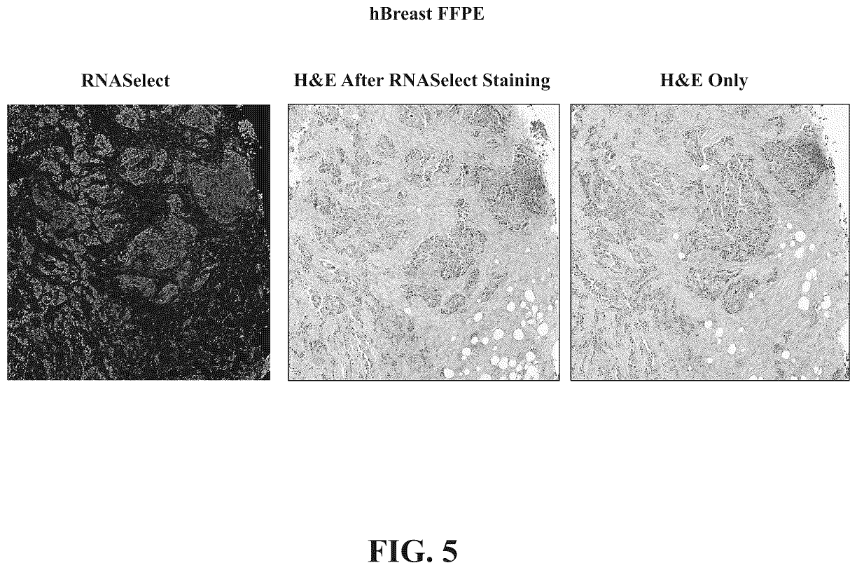

Some de-paraffinized tissue sections were additionally de-crosslinked using a catalyst in a buffer solution. The de-paraffinized tissue sections and/or the de-paraffinized/de-crosslinked tissue sections were blocked for 30 minutes at room temperature (RT) and then stained with phalloidin in blocking buffer (e.g., 1:250 or 1:200 dilution) and with SYTO™ RNASelect™ (e.g., 250 nM or 500 nM). The tissue sections were also stained with DAPI (5 pg/mL) for 1 minute and/or with RNA IQ (1:200 dilution) for 2 minutes. The tissue sections were imaged using fluorescent microscopy. Human FFPE tissue samples tested included samples from human lung (FIGS. 3A-3C), brain (FIG. 4), and breast (FIG. 5).

For analyte detection, circularizable probes (e.g., padlock probes) targeting various RNA transcripts were added in hybridization buffers (e.g., including SSC and formamide) and incubated with the samples to allow hybridization of the circularizable probes to their target nucleic acids. In addition to a target hybridization region, each circularizable probe also contained a common anchor region (e.g., “Anchor” in FIG. 4) and a barcode region. Then, the probe hybridization mixture was removed and the samples were washed. For ligation of the circularizable probes hybridized to their target nucleic acids, a ligation reaction mix (e.g., containing a SplintR® ligase buffer, RNase inhibitor and SplintR® ligase) and a rolling circle amplification (RCA) primer were added to the samples and incubated for probe circularization and RCA primer hybridization to the probes. The samples were washed and an RCA reaction mixture (containing Phi29 reaction buffer, dNTPs, Phi29 polymerase) was added and incubated for RCA of the circularized probes. The samples were then washed (e.g., in PBST) and detectable probes in a hybridization buffer (e.g., containing SSC and formamide) were hybridized to RCA products (RCPs) in the sample. The detectable probes included probes that hybridize to sequences (e.g., barcode sequences or anchor sequences) in the RCPs and comprise overhangs for hybridization of fluorescently labelled detection oligonucleotides. The samples were imaged using fluorescent microscopy and the signals associated with the RCPs were quantified using a software.

FIG. 3A shows representative images of human lung (hLung) FFPE tissue sections from two tissue blocks, Block 1 and Block 2. Tissue sections from the two blocks were either de-paraffinized (“DP”) or de-paraffinized/de-crosslinked (“DXL”) and then stained with DAPI, RNASelect™, and phalloidin. The RNASelect™ nucleus/cytoplasm signal-to-noise ratios (SNRs) are plotted in FIG. 3B. The RNASelect™ nucleus/cytoplasm SNR was calculated as the ratio between signal intensity in a nucleus and signal intensity of the cytoplasm around the nucleus. Similar results as shown in FIG. 3B were obtained when the nucleus/cytoplasm SNR was calculated using an automated pipeline that calculated the intensity in the nucleus and the cytoplasm.

RNASelect™ nucleus/cytoplasm SNR values were in general higher in Block 1 tissue sections than in Block 2 tissue sections (FIG. 3B), which was consistent with the results of RCA based analyte detection showing fewer detected RCPs in Block 2 tissue sections (FIG. 3C). Thus, the RNASelect™ nucleus/cytoplasm SNR value can be correlated with poor sample preparation and quality, as well as with the number of detected RCPs. Since RCP detection can be compromised in poor quality tissue samples, RNASelect™ staining can be used as a quality control parameter for subsequent RCP detection, and/or as a predictor of the number of detected RCPs.

FIG. 4 shows representative images of human brain (hBrain) FFPE tissue sections fixed for 4 hours, 24 hours, or 10 days. RCPs associated with various target nucleic acids and the signals associated with the anchor sequence are shown. RNASelect™ and phalloidin were used to assess sample quality prior to circularizable probe hybridization to the samples and RCP detection. Signals associated with RNASelect™ and phalloidin were not detected when detecting the signals associated with the RCPs. In particular, although phalloidin was detectable in the same fluorescent channel as RCPs associated with MBP, PLP1, and SOX10, signals associated with phalloidin were not detected in the fluorescent channel. No separate removing step was used to remove RNASelect™ or phalloidin prior to RCP detection, and RNASelect™ and phalloidin staining prior to library preparation (e.g., circularizable probe hybridization, ligation, and RCA) did not negatively impact subsequent in situ detection of RNA transcripts.

FIG. 5 shows representative images of human breast (hBreast) FFPE tissue sections stained with RNASelect™, with H&E after RNASelect™ staining, and with H&E staining only (that is, without prior RNASelect™ staining of the same tissue section). These results indicate that H&E staining was not negatively impacted by prior RNASelect™ staining. Thus, a fixed biological sample can be de-paraffinized (if necessary), stained with a nucleic acid stain such as RNASelect™ and/or an actin stain, imaged to assess sample quality, and then stained with H&E for sample morphology. The sample can be optionally de-crosslinked or additionally fixed prior to analyte detection.

### Example 4: Assessing Quality of Human FFPE Liver and Mouse Pancreas Samples

Human FFPE liver tissue samples were cut into tissue sections of approximately 5 μm in thickness and placed on slides. The slides from two samples (Sample 1 and Sample 2) were de-paraffinized, de-crosslinked and processed as described substantially in Example 3. The de-paraffinized tissue sections and/or the de-paraffinized/de-crosslinked tissue sections were blocked for 30 minutes at room temperature (RT) and then stained with different RNA dyes (Cell Navigator® RNA dye for 20 minutes and QuantiFluor® RNA dye for 10 minutes). The tissue sections were also stained with DAPI (5 pg/mL) for 1 minute. The tissue sections were imaged using fluorescent microscopy.

For analyte detection, circularizable probes (e.g., padlock probes) targeting various RNA transcripts (a gene panel composed of low, medium, and higher housekeeping genes) were added in hybridization buffers (e.g., including SSC and formamide) and incubated with the samples to allow hybridization of the circularizable probes to their target nucleic acids. Then, the probe hybridization mixture was removed and the samples were washed. Ligation, amplification, and detection of RCPs (via a common anchor region in each circularizable probe) was performed substantially as described in Example 3.

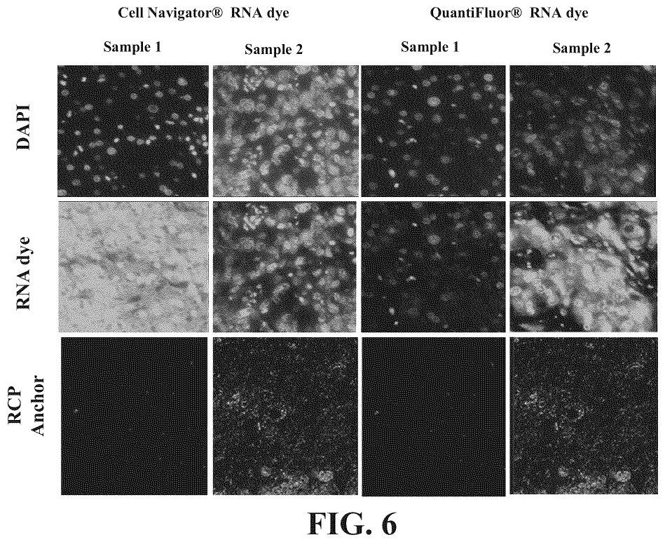

FIG. 6 shows representative images of human liver FFPE tissue sections from two tissue blocks, Sample 1 and Sample 2. RNA dye signal intensity was generally higher in Sample 2 tissue samples compared to Sample 1. These results were consistent with the results of RCA based analyte detection showing fewer detected RCPs in Sample 1 compared to Sample 2. Thus, both RNA dyes can be correlated with the quality of the samples and correlated with downstream analyte detection e.g., as a predictor of the number of detected RCPs. For example, the RNA dye intensity of Sample 2 can be correlated with good sample preparation and tissue quality associated with a greater number of detected RCPs. In addition, similar results were obtained using the dye (E)-2-(2-(1H-indol-2-yl)vinyl)-1-methyl-4-((2-(piperidin-1-yl)ethyl)amino) quinolin-1-ium iodide.

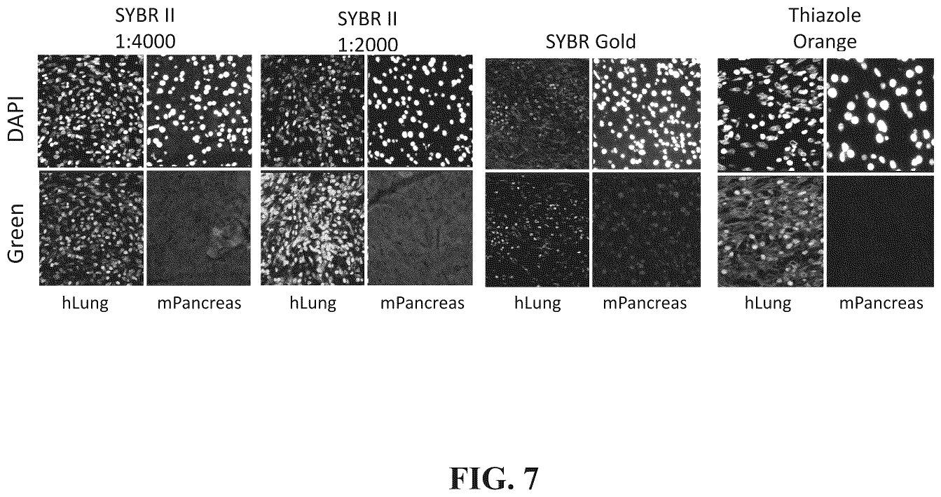

Separately, human FFPE liver tissue sections and mouse FFPE pancreas tissue samples were prepared substantially as described above and then stained with different dyes to compare staining with SYBR™ Green II dye at a 1:4,000 dilution, SYBR™ Green II dye at a 1:2,000 dilution, SYBR™ Gold dye, and a Thiazole Orange dye. Results for the indicated tissue samples in FIG. 7 show that SYBR™ Green II staining showed specific signals and can be used to stain different tissue types. Similar staining has been performed in heart, kidney, liver, lung, lymph node, pancreas, skin, brain, lung, and colon tissue samples to assess sample quality.

### Example 5: Regional Assessment of Tissue Sample Quality

Human FFPE liver tissue sections were prepared substantially as described in Example 4 and then stained with different nucleic dyes (Cell Navigator® RNA dye for 20 minutes and SYBR™ Green II dye for 10 minutes). The tissue sections were imaged using fluorescent microscopy and staining in various regions of the same tissue sample were compared.

The nucleus/cytoplasm SNR values for both tested dyes were in general higher in Sample Region 1 compared to Sample Region 2 (FIG. 8A) in the same tissue section, which was consistent with the results of RCA based analyte detection showing fewer detected RCPs in Sample Region 2 (about 69.5 transcripts per cell were detected) compared to Sample Region 1 (about 178.5 transcripts per cell were detected). Thus, the nucleus/cytoplasm SNR value derived from staining using both dyes can be correlated with poor sample preparation and/or quality, as well as with the number of detected RCPs.

To further determine if the tissue sample has any regional differences, such as areas of low quality cells or analytes (e.g., RNA), a method to bin the SNR scores of the cells in each region is used to provide a quality map for the tissue sample. As shown in FIG. 8B, the tissue sample is divided into regions (e.g., rectangular regions) and within each rectangular region, nucleic acid stain results are determined. For example, the intensity of stain(s), SNR scores, or Spearman correlation with DAPI stain can be averaged for each rectangular region. For example, a region may be considered to have a satisfactory percentage of good quality cells for downstream analysis if more than 70% of cells in the region shows staining indicative of good quality (e.g., the upper left portion of the dashed line in FIG. 8B). In some cases, a region may be considered to not have a satisfactory percentage of good quality cells for downstream analysis if more than 50% in the region show staining indicative of poor quality (e.g., the bottom right portion of the dashed line in FIG. 8B). The threshold based on binning SNR or intensity of signals can be adjusted and determined based on data for the outcome of downstream assays. Spearman correlation of the nucleic acid stain with DAPI stain was performed and the correlation was observed to be higher in Sample Region 1 compared to Sample Region 2.

A display of the tissue sample is provided to the user to indicate whether each boxed region contains analytes and/or cells of good quality or poor quality. A user may then include or exclude data from certain regions based on this analysis. In some cases, the regions of poor quality may be correlated with low assay performance metrics, poor cell clustering, or lower median transcripts per cell in a assay for detecting RNA transcripts. For example, a transcript density map of a human liver tissue sample in FIG. 8B (same sample as described in Example 5 with Sample Region 1 and Sample Region 2) was obtained by performing analyte detection substantially as described in Example 4 using circularizable probes (e.g., padlock probes) targeting various RNA transcripts. Results showed that within this tissue sample, a portion of the tissue sample showed an abundance of detected transcripts while another portion showed few detected transcripts (FIG. 8B right panel).

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-118. (canceled)

119. A method for sample analysis, comprising:
a) contacting a section of a fixed biological sample with a ribonucleic acid (RNA) stain;
b) detecting an optical signal associated with the nucleic acid stain in the section of the fixed biological sample, wherein the optical signal associated with the nucleic acid stain is detected in the nucleus; and
c) comparing the optical signal(s) detected in b) to a reference to determine the quality of the fixed biological sample, wherein the comparing comprises using a ratio between the optical signal associated with the nucleic acid stain detected in the nucleus and an optical signal associated with background signal detected in a cytoplasm.

120. The method of claim 119, further comprising de-crosslinking or additionally fixing the fixed biological sample to adjust the level of fixation.

121. The method of claim 119, further comprising contacting the section of the fixed biological sample or an additional portion of the fixed biological sample with a nucleic acid probe that directly or indirectly binds to an analyte or a product thereof in the fixed biological sample.

122. The method of claim 119, further comprising adjusting the level of fixation of an additional biological sample, wherein the additional biological sample is contacted with a nucleic acid probe that directly or indirectly binds to an analyte or a product thereof in the additional biological sample.

123. The method of claim 119, wherein the reference comprises one or more reference images or reference samples.

124. The method of claim 119, wherein the nucleic acid stain is a cyanine dye.

125. The method of claim 119, wherein the fixed biological sample is fixed using a fixing composition which optionally comprises 0.01-100% of a fixative selected from the group consisting of: formaldehyde, glutaraldehyde, acetone, methanol, ethanol, acetic acid, potassium dichromate, chromic acid, potassium permanganate, B-5, Zenker's fixative, uranyl acetate, mercuric chloride, osmium tetroxide, potassium permanganate, and 1-ethyl-3-(3-dimethylamino propyl)carbodiimide (EDC), picric acid, glyoxal, bis(sulfosuccinimidyl)suberate, and derivatives thereof.

126. The method of claim 119, wherein the fixed biological sample is de-paraffinized prior to the contacting in a).

127. The method of claim 119, wherein the fixed biological sample is de-crosslinked after the comparing in c).

128. The method of claim 119, wherein the fixed biological sample is additionally fixed after the comparing in c), wherein the additional fixation comprises contacting the fixed biological sample with a crosslinking agent.

129. The method of claim 119, wherein the fixed biological sample is contacted with an additional nucleic acid stain that binds to DNA or a cytoplasm stain for determining the location of the nucleus.

130. The method of claim 129, wherein the additional nucleic acid stain comprises DAPI.

131. The method of claim 121, comprising detecting an optical signal associated with the nucleic acid probe or a product thereof at a location of a cell or an additional nucleus in the fixed biological sample or the additional portion of the fixed biological sample, thereby detecting the analyte at the location in the fixed biological sample.

132. The method of claim 121, comprising partitioning a cell or an additional nucleus in the additional portion of the fixed biological sample in a partition comprising a partition barcode.

133. The method of claim 132, wherein the partition is an emulsion droplet or a microwell.

134. The method of claim 132, comprising sequencing a nucleic acid molecule or a portion thereof comprising i) a sequence of the nucleic acid probe or a complement thereof and ii) the partition barcode or a complement thereof.

135. The method of claim 119, wherein the fixed biological sample is a formalin-fixed paraffin-embedded (FFPE) biological sample.

136. The method of claim 119, wherein the analyte is an mRNA, and a first nucleic acid probe and a second nucleic acid probe are hybridized to a first analyte sequence and a second analyte sequence in the mRNA, respectively, wherein:
the first nucleic acid probe comprises: i) a first hybridization region complementary to the first analyte sequence, and ii) a first overhang;
the second nucleic acid probe comprises: i) a second hybridization region complementary to the second analyte sequence, and ii) a second overhang;
the first and second nucleic acid probes are ligated using the mRNA as template to form a ligated nucleic acid probe, with or without gap filling prior to the ligation; and
the first and second overhangs independently comprise a primer binding sequence, a capture sequence, a barcode sequence, and/or a constant sequence.

137. The method of claim 136, further comprising transferring the ligated first and second nucleic acid probes to an array of features on a substrate, each of which comprises a spatial barcode sequence associated with a unique spatial location on the array and generating a nucleic acid molecule comprising i) the spatial barcode sequence or a complement thereof and ii) a sequence of the ligated first and second nucleic acid probes, or a complement thereof.

138. The method of claim 137, comprising determining a sequence of the nucleic acid molecule, thereby determining the spatial location of the ligated first and second nucleic acid probes in the biological sample.
